(19) United States
(12) Patent Application Publication
(10) Pub. No.: US 2008/0280860 A1
(43) Pub. Date: Nov. 13, 2008

(54) Use of Vitamin D Compounds to Treat Endometriosis

(75) Inventor: Paola Panina, Milano (IT)

Correspondence Address: EDWARDS ANGELL PALMER & DODGE LLP, P.O. BOX 55874, BOSTON, MA, 02205, US

(73) Assignee: BioXell S.p.A., Milan (IT)

(21) Appl. No.: 11/885,608

(22) PCT Filed: Mar. 23, 2006
(86) PCT No.: PCT/EP2006/060983

§ 371 Date: Jun. 20, 2008

Related U.S. Application Data
(60) Provisional application No. 60/667,367, filed on Mar. 31, 2005.

(30) Foreign Application Priority Data
Mar. 23, 2005 (GB) 0505954.8

(51) Int. Cl. A61K 31/59; A61P 15/00

(52) U.S. Cl. 514/167

(57) ABSTRACT

The use of vitamin D compounds in the treatment or prevention of endometriosis, methods for the treatment or prevention of endometriosis by administering a vitamin D compound, and compounds for use therein.

This application claims the benefit of GB 0505955.5, filed 23 Mar. 2005, and U.S. provisional application Ser. No. 60/667,367, filed 31 Mar. 2006, the disclosures of which applications are incorporated herein by this reference.

The present invention relates to the use of vitamin D compounds in the treatment or prevention of endometriosis, methods for the treatment or prevention of endometriosis by administering a vitamin D compound, and compounds for use therein.

Endometriosis is a disease which involves the growth of endometrium at ectopic sites that results in sub-fertility, chronic pelvic pain and multiple surgeries. It affects approx. 10% of the female population in their reproductive years (Balweg, M. (2004) Best Pract. Res. Cl. Ob. 18:201 and Vigano, P. et al 2004) Best Pract. Res. Cl. Ob. 18:177). Proliferation of stromal cells, vascular development and inflammation are important factors in the pathogenesis of endometriosis (Kayama, C. M (2003) Reproductive Biology and Endocrinology 1:123). Most of the current medical therapies involve inducing a hypoestrogenic state in patients. Those treatments are associated with severe side effects and high recurrence rates of the disease.

The present inventors have developed a new method of treating endometriosis with a view to mitigating or alleviating the aforementioned disadvantages.

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorus homeostasis.

Studies involving the metabolism of vitamin D₃ were initiated with the discovery and chemical characterization of the plasma metabolite, 25-hydroxyvitamin D_{3 [}25(OH) D₃] (Blunt, J. W. et al. (1968) Biochemistry 6:3317-3322) and the hormonally active form, 1-alpha,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1-alpha,25(OH)₂D₃ in a carefully regulated fashion (Fraser, D. R. and Kodicek, E (1970) Nature 288:764-766; Wong, R. G. et al. (1972) J. Clin. Invest 51:1287-1291), and the discovery of a nuclear receptor for 1-alpha,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1-alpha,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1-alpha,25(OH)₂D₃ to generate the requisite specific biological responses for this secosteroid hormone (Pike, J. W. (1991) Annu. Rev. Nutr. 11:189-216). To date, there is evidence that nuclear receptors for 1-alpha,25(OH)₂D₃ (VD₃R) exist in more than 30 tissues and cancer cell lines (Reichel, H. and Norman, A. W. (1989) Annu. Rev. Med. 40:71-78), including the normal bladder.

Vitamin D₃ and its hormonally active forms are well-known regulators of calcium and phosphorus homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1-alpha,25(OH)₂ D₃ has been suggested by the combined presence of enzymes capable of oxidizing vitamin D₃ into its active forms, e.g., 25-OHD-1-alpha-hydroxylase, and specific receptors in several issues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40: 71-78).

Given the activities of vitamin D₃ and its metabolites, much attention has focused on the development of synthetic analogues of these compounds. A large number of these analogues involve structural modifications in the A ring, B ring, C/D rings, and, primarily, the side chain (Bouillon, R. et al. (1995) Endocrine Reviews 16(2):201-204). Although a vast majority of the vitamin D₃ analogues developed to date involve structural modifications in the side chain, a few studies have reported the biological profile of A-ring diastereomers (Norman, A. W. et al. (1993) J. Biol. Chem. 268 (27): 20022-20030). Furthermore, biological esterification of steroids has been studied (Hochberg, R. B., (1998) Endocr. Rev. 19(3): 331-348), and esters of vitamin D₃ are known (WO 97/11053).

Moreover, despite much effort in developing synthetic analogues, clinical applications of vitamin D and its structural analogues have been limited by the undesired side effects elicited by these compounds after administration to a subject for known indications/applications of vitamin D compounds.

The activated form of vitamin D, vitamin D₃, and some of its analogues have been described as potent regulators of cell growth and differentiation. It has previously been found that vitamin D₃ as well as an analogue (analogue V), inhibited BPH cell proliferation and counteracted the mitogenic activity of potent growth factors for BPH cells, such as keratinocyte growth factor (KGF) and insulin-like growth factor (IGF1). Moreover, the analogue induced bcl-2 protein expression, intracellular calcium mobilization, and apoptosis in both unstimulated and KGF-stimulated BPH cells.

Ailawadi et al Fertil. Steril. 2004 81 (2):290-296 describes the treatment of endometriosis and chronic pelvic pain with letrozole and norethindrone acetate. A range of additional medicaments, including calcium and vitamin D supplements were provided to reduce possible treatment associated bone loss.

Shippen et al Fertil. Steril. 2004 81(5):1395-1398 describes the treatment of severe endometriosis with an aromatase inhibitor. A range of additional medicaments were provided, including calcitriol primarily to reduce bone loss potential.

US2005/0032741 discloses vitamin compositions containing calcium, vitamin D, folic acid, vitamin B12 and vitamin B6 for the treatment or prevention of conditions associated with hormonal changes in an individual. In one example, a patient suffering from endometriosis and osteoporosis, concurrently receiving a gonadotropin releasing hormone antagonist, Leuprolide and Fosamax, showed a decrease in rate of bone loss and endometriosis when the vitamin composition was administered. In light of the number of agents administered in combination, there is no evidence that the reduction in endometriosis symptoms was a direct result of vitamin D administration.

US2002/0010163 discloses novel vitamin D compounds. Said compounds are stated to be of use as antiproliferative agents, for example in the treatment of hormone responsive tumours or hyperplasias (such as breast, prostate or ovarian cancers, fibroids or endometriosis), or as suppressants of progesterone activity, for instance in oedema, acne, melasma or fertility control. No biological data is provided in the application for any of the stated indications.

Thus the invention provides vitamin D compounds, and new methods of treatment using such compounds, for the prevention or treatment of endometriosis, and associated symptoms e.g. chronic pelvic pain and/or sub-fertility. Treatment and/or prevention may include a reduction in the number and/or size of ectopic growths. In one embodiment the use and methods of the present invention may relate to adenomyosis (also known as endometriosis interna, uterine endometriosis or internal endometriosis).

Suitably the methods of the present invention may be applied to the treatment of endometriosis. Alternatively, the methods of the present invention may be applied to the prevention of endometriosis.

Before further description of the present invention, and in order that the invention may be more readily understood, certain terms are first defined and collected here for convenience.

The term “administration” or “administering” includes routes of introducing the vitamin D compound(s) to a subject to perform their intended function. Examples of routes of administration which can be used include injection (subcutaneous, intravenous, parenterally, intraperitoneally), oral, inhalation, rectal, transdermal or via bladder instillation. The pharmaceutical preparations are, of course, given by forms suitable for each administration route. For example, these preparations are administered in tablets or capsule form, by injection, infusion, inhalation, lotion, ointment, suppository, etc. Oral administration is preferred. The injection can be bolus or can be continuous infusion. Depending on the route of administration, the vitamin D compound can be coated with or disposed in a selected material to protect it from natural conditions which may detrimentally affect its ability to perform its intended function. The vitamin D compound can be administered alone, or in conjunction with either another agent of use in the treatment of endometriosis, or with a pharmaceutically-acceptable carrier, or both. The vitamin D compound can be administered prior to the administration of the other agent, simultaneously with the agent, or after the administration of the agent. Furthermore, the vitamin D compound can also be administered in a pro-form which is converted into its active metabolite, or more active metabolite in vivo.

The term “effective amount” includes an amount effective, at dosages and for periods of time necessary, to achieve the desired result, i.e. sufficient to treat and/or to prevent endometriosis. An effective amount of vitamin D compound may vary according to factors such as the disease state, age and weight of the subject, and the ability of the vitamin D compound to elicit a desired response in the subject. Dosage regimens may be adjusted to provide the optimum therapeutic response. An effective amount is also one in which any toxic or detrimental effects (e.g., side effects) of the vitamin D compound are outweighed by the therapeutically beneficial effects.

A therapeutically effective amount of vitamin D compound (i.e., an effective dosage) may range from about 0.001 to 30 ug/kg body weight, preferably about 0.01 to 25 ug/kg body weight, more preferably about 0.1 to 20 ug/kg body weight, and even more preferably about 1 to 10 ug/kg, 2 to 9 ug/kg, 3 to 8 ug/kg, 4 to 7 ug/kg, or 5 to 6 ug/kg body weight. The skilled artisan will appreciate that certain factors may influence the dosage required to effectively treat a subject, including but not limited to the severity of the disease or disorder, previous treatments, the general health and/or age of the subject, and other diseases present. In addition, the dose administered will also depend on the particular vitamin D compound used, the effective amount of each compound can be determined by titration methods known in the art. Moreover, treatment of a subject with a therapeutically effective amount of a vitamin D compound can include a single treatment or, preferably, can include a series of treatments. In one example, a subject is treated with a vitamin D compound in the range of between about 0.1 to 20 ug/kg body weight, one time per day for a duration of six months or longer, depending on management of the symptoms and the evolution of the condition. Also, as with other chronic treatments an “on-off” or intermittent treatment regime can be considered. It will also be appreciated that the effective dosage of a vitamin D compound used for treatment may increase or decrease over the course of a particular treatment.

The term “alkyl” refers to the radical of saturated aliphatic groups, including straight-chain alkyl groups, branched-chain alkyl groups, cycloalkyl (alicylic) groups, alkyl substituted cycloalkyl groups, and cycloalkyl substituted alkyl groups. The term alkyl further includes alkyl groups, which can optionally further include (for example, in one embodiment alkyl groups do not include) oxygen, nitrogen, sulfur or phosphorus atoms replacing one or more carbons of the hydrocarbon backbone, e.g., oxygen, nitrogen, sulfur or phosphorus atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, especially 6 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). Unsubstituted alkyl (including cycloalkyl) groups or groups substituted by halogen, especially fluorine, are generally preferred over other substituted groups. The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, propyl (n-propyl and i-propyl), butyl (tert-butyl, n-butyl and sec-butyl), pentyl, hexyl, heptyl, octyl and so forth. In preferred embodiment, the term “lower alkyl” includes a straight chain alkyl having 4 or fewer carbon atoms in its backbone, e.g., C₁-C₄ alkyl.

Thus specific examples of alkyl include C₁₋₆ alkyl or C₁₋₄alkyl (such as methyl or ethyl). Specific examples of hydroxyalkyl include C₁₋₆hydroxyalkyl or C₁₋₄hydroalkyl (such as hydroxymethyl).

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The terms “alkenyl” and “alkynyl” refer to unsaturated aliphatic groups analogueous in length and possible substitution to the alkyls described above, but that contain at least one double or triple bond, respectively. For example, the invention contemplates cyano and propargyl groups.

The term “chiral” refers to molecules which have the property of non-superimposability of the mirror image partner, while the term “chiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

As used herein, the term “halogen” designates —F, —Cl, —Br or —I; the term “sulfhydryl” or “thiol” means —SH; the term “hydroxyl” means —OH.

The term “haloalkyl” is intended to include alkyl groups as defined above that are mono-, di- or polysubstituted by halogen, e.g., C₁₋₆haloalkyl or C₁₋₄haloalkyl such as fluoromethyl and trifluoromethyl.

The term “heteroatom” as used herein means an atom of any element other than carbon or hydrogen. Preferred heteroatoms are nitrogen, oxygen, sulfur and phosphorus.

The terms “polycyclyl” or “polycyclic radical” refer to the radical of two or more cyclic rings (e.g., cycloalkyls, cycloalkenyls, cycloalkynyls, aryls and/or heterocydyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “isomers” or “stereoisomers” refers to compounds which have identical chemical constitution, but differ with regard to the arrangement of the atoms or groups in space.

The terms “isolated” or “substantially purified” are used interchangeably herein and refer to vitamin D₃ compounds in a non-naturally occurring state. The compounds can be substantially free of cellular material or culture medium when naturally produced, or chemical precursors or other chemicals when chemically synthesized. In one embodiment of the invention an isolated vitamin D compound is at least 75% pure, especially at least 85% pure, in particular at least 95% pure and preferably at least 99% pure on a w/w basis, said purity being by reference to compounds with which the vitamin D compound is naturally associated or else chemically associated in the course of chemical synthesis. In certain preferred embodiments, the terms “isolated” or “substantially purified” also refer to preparations of a chiral compound which substantially lack one of the enantiomers; i.e., enantiomerically enriched or non-racemic preparations of a molecule. Similarly, the terms “isolated epimers” or “isolated diastereomers” refer to preparations of chiral compounds which are substantially free of other stereochemical forms. For instance, isolated or substantially purified vitamin D₃ compounds include synthetic or natural preparations of a vitamin D₃ enriched for the stereoisomers having a substituent attached to the chiral carbon at position 3 of the A-ring in an alpha-configuration, and thus substantially lacking other isomers having a beta-configuration. Unless otherwise specified, such terms refer to vitamin D₃ compositions in which the ratio of alpha to beta forms is greater than 1:1 by weight. For instance, an isolated preparation of an a epimer means a preparation having greater than 50% by weight of the alpha-epimer relative to the beta stereoisomer, more preferably at least 75% by weight, and even more preferably at least 85% by weight. Of course the enrichment can be much greater than 85%, providing “substantially epimer-enriched” preparations, i.e., preparations of a compound which have greater than 90% of the alpha-epimer relative to the beta stereoisomer, and even more preferably greater than 95%. The term “substantially free of the beta stereoisomer” will be understood to have similar purity ranges.

As used herein, the term “vitamin D compound” includes any compound being an analogue of vitamin D that is capable of treating or preventing endometriosis. Generally, compounds which are ligands for the Vitamin D receptor (VDR ligands) and which are capable of treating or preventing endometriosis are considered to be within the scope of the invention. Vitamin D compounds are preferably agonists of the vitamin D receptor. Thus, vitamin D compounds are intended to include secosteroids. Examples of specific vitamin D compounds suitable for use in the methods of the present invention are further described herein. A vitamin D compound includes vitamin D₂ compounds, vitamin D₃ compounds, isomers thereof, or derivatives/analogues thereof. Preferred vitamin D compounds are vitamin D₃ compounds which are ligands of (more preferably are agonists of) the vitamin D receptor. Preferably the vitamin D compound (e.g., the vitamin D₃ compound) is a more potent agonist of the vitamin D receptor than the native ligand (i.e. the vitamin D, e.g., vitamin D₃). Vitamin D compounds, vitamin D₂ compounds and vitamin D₃ compounds include, respectively, vitamin D₁, D₂, D₃ and analogues thereof. In certain embodiments, the vitamin D compound may be a steroid, such as a secosteroid, e.g., calciol, calcidiol or calcitriol. Non-limiting examples of vitamin D compounds in accordance with the invention include those described in U.S. Pat. Nos. 6,017,908, 6,100,294, 6,030,962, 5,428029 and 6,121,312, published international applications WO 98/51633, WO 01/40177A3. Other examples of vitamin D compounds include those described in U.S. Pat. No. 6,492,353 and WO2005/030222.

The term “secosteroid” is art-recognized and includes compounds in which one of the cyclopentanoperhydro-phenanthrene rings of the steroid ring structure is broken. For example, 1-alpha,25(OH)₂D₃ and analogues thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1alpha,25(OH)₂D₃ is illustrated herein having all carbon atoms numbered using standard steroid notation.

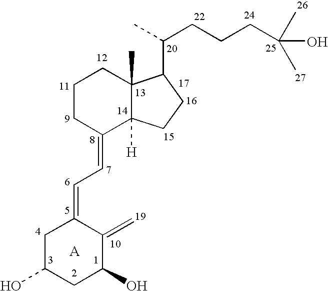

In the formulas presented herein, the various substituents on ring A are illustrated as joined to the steroid nucleus by one of these notations: a dotted line (----) indicating a substituent which is in the beta-orientation (i.e., above the plane of the ring), a wedged solid line ( [symbol] ) indicating a substituent which is in the alpha-orientation (i.e., below the plane of the molecule), or a wavy line [symbol] indicating that a substituent may be either above or below the plane of the ring. In regard to ring A, it should be understood that the stereochemical convention in the vitamin D field is opposite from the general chemical field, wherein a dotted line indicates a substituent on Ring A which is in an alpha-orientation (i.e., below the plane of the molecule), and a wedged solid line indicates a substituent on ring A which is in the beta-orientation (i.e., above the plane of the ring).

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention.

As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “carbon centers.”

With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

Also, throughout the patent literature, the A ring of a vitamin D compound is often depicted in generic formulae as any one of the following structures:

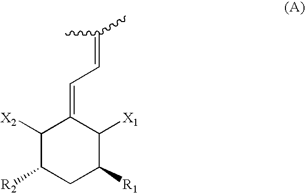

wherein X₁ and X₂ are defined as H or ═CH₂; or

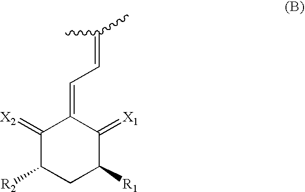

wherein X₁ and X₂ are defined as H₂ or CH₂.

Although there does not appear to be any set convention, it is clear that one of ordinary skill in the art understands either formula (A) or (B) to represent an A ring in which, for example, X₁ is ═CH₂ and X₂ is defined as H₂, as follows:

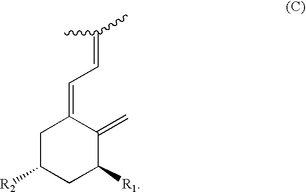

For purposes of the instant invention, formula (B) will be used in all generic structures.

Thus, in one aspect, the invention provides the use of a Vitamin D compound in the prevention or treatment of endometriosis. Also provided is a method of treating a patent with endometriosis by administering an effective amount of a Vitamin D compound. Further provided is the use of a Vitamin D compound in the manufacture of a medicament for the prevention or treatment of endometriosis. Further provided is a vitamin D compound for use in the prevention and/or treatment of endometriosis. Also provided is a kit containing a vitamin D compound together with instructions directing administration of said compound to a patient in need of treatment and/or prevention of endometriosis thereby to treat and/or prevent endometriosis in said patient. Endometriosis may, for example, be characterized by the presence of symptoms of chronic pelvic pain and/or sub-fertility.

The uses and methods are uses and methods in the treatment of human females, especially pre-menopausal human females.

In one embodiment of the invention, the vitamin D compound is a compound of formula (I):

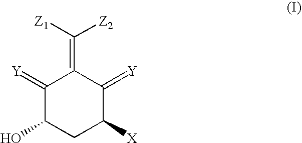

wherein:
X is hydroxyl or fluoro;
Y is H₂ or CH₂;
Z₁ and Z₂ are H or a substituent represented by formula (II), provided Z₁ and Z₂ are different (preferably Z₁ and Z₂ do not both represent formula (II))

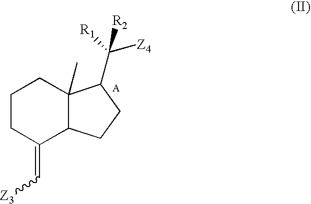

wherein:
Z₃ represents the above-described formula (I);
A is a single bond or a double bond;
R₁, R₂, and Z₄, are each, independently, hydrogen, alkyl, or a saturated or unsaturated carbon chain represented by formula (III), provided that at least one of R₁, R₂, and Z₄ is the saturated or unsaturated carbon chain represented by formula (III) and provided that all of R₁, R₂, and Z₄ are not saturated or unsaturated carbon chain represented by formula (III):

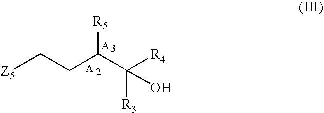

wherein:
Z₅ represents the above-described formula (II);
A₂ is a single bond, a double bond, or a triple bond; and
A₃ is a single bond or a double bond; and
R₃, and R₄, are each, independently, hydrogen, alkyl, haloalkyl, hydroxyalkyl; and R₅ is H₂ or oxygen. R₅ may also represent hydrogen or may be absent.

Thus, in the above structure of formula (III) (and in corresponding structures below), when A₂ represents a triple bond R₅ is absent. When A₂ represents a double bond R₅ represents hydrogen. When A₂ represents a single bond R₅ represents a carbonyl group or two hydrogen atoms.

In another embodiment of the invention, the vitamin D compound is a compound of formula (IV):

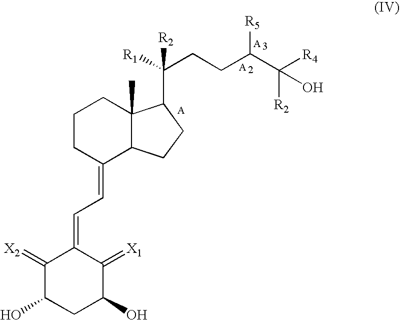

wherein:
X₁ and X₂ are H₂ or CH₂, wherein X₁ and X₂ are not CH₂ at the same time;
A is a single or double bond;
A₂ is a single, double or triple bond;
A₃ is a single or double bond;
R₁ and R₂ are hydrogen, C₁-C₄ alkyl or 4-hydroxy-4-methylpentyl, wherein R₁ and R₂ are not both hydrogen;
R₅ is H₂ or oxygen, R₅ may also represent hydrogen or may be absent;
R₃ is C₁-C₄ alkyl, hydroxyalkyl or haloalkyl, e.g., fluoroalkyl, e.g., fluoromethyl and trifluoromethyl; and
R₄ is C₁-C₄ alkyl, hydroxyalkyl or haloalkyl, e.g., fluoroalkyl, e.g., fluoromethyl and trifluoromethyl.

In yet another embodiment of the invention, the vitamin D compound is a compound of formula

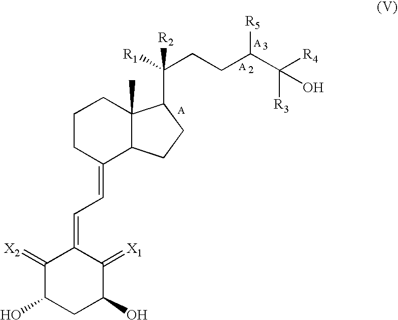

wherein:
X₁ and X₂ are H₂ or CH₂, wherein X₁ and X₂ are not CH₂ at the same time;
A is a single or double bond;
A₂ is a single, double or triple bond;
A₃ is a single or double bond;
R₁ and R₂ are hydrogen, C₁-C₄ alkyl, wherein R₁ and R₂ are not both hydrogen;
R₅ is H₂ or oxygen, R₅ may also represent hydrogen or may be absent;
R₃ is C₁-C₄ alkyl, hydroxyalkyl or haloalkyl, e.g., fluoroalkyl, e.g., fluoromethyl and trifluoromethyl; and
R₄ is C₁-C₄ alkyl, hydroxyalkyl haloalkyl, e.g., or fluoroalkyl, e.g., fluoromethyl and trifluoromethyl.

An example of the above structure of formula (V) is 1,25-dihydroxy-16-ene-23-yne cholecalciferol.

In another embodiment, the vitamin D compound is a compound of formula VII):

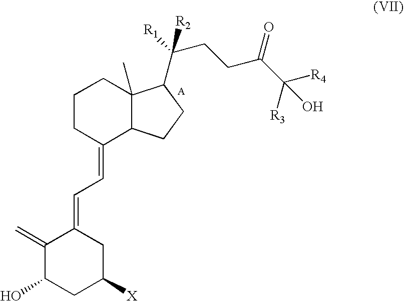

wherein:
A is a single or double bond;
R₁ and R₂ are each, independently, hydrogen, alkyl (for example methyl);
R₃, and R₄, are each, independently, alkyl, and
X is hydroxyl or fluoro.

In a further embodiment, the vitamin D compound is a compound having formula (VIII):

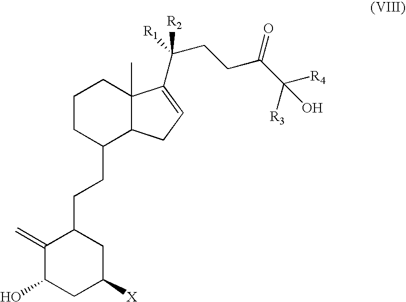

wherein:
R₁ and R₂, are each, independently, hydrogen, or alkyl, e.g., methyl;
R₃ is alkyl, e.g., methyl,
R₄ is alkyl, e.g., methyl; and
X is hydroxyl or fluoro.

In specific embodiments of the invention, the vitamin D compound is selected from the group consisting of:

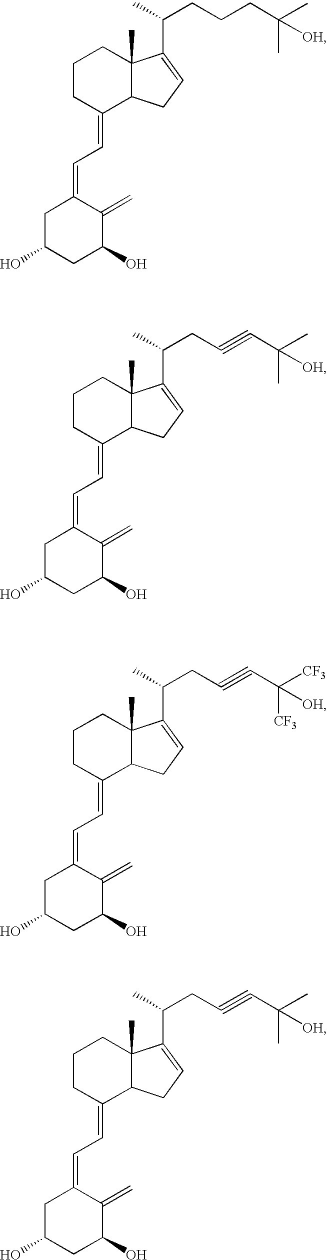

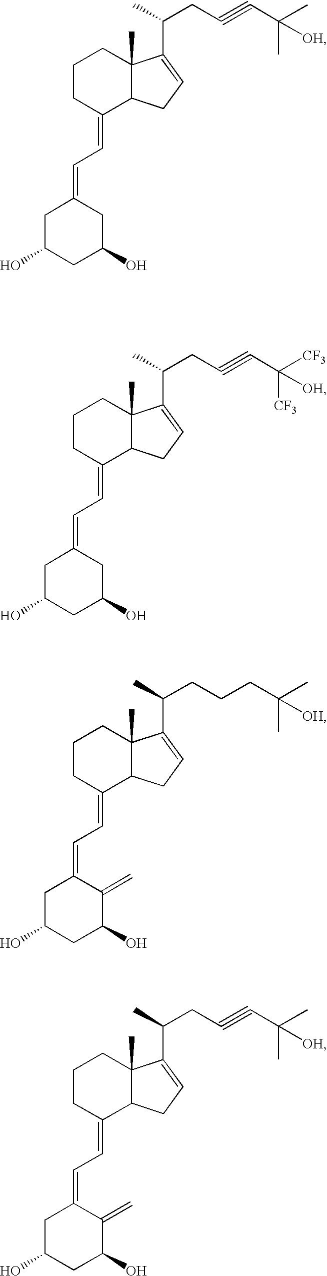

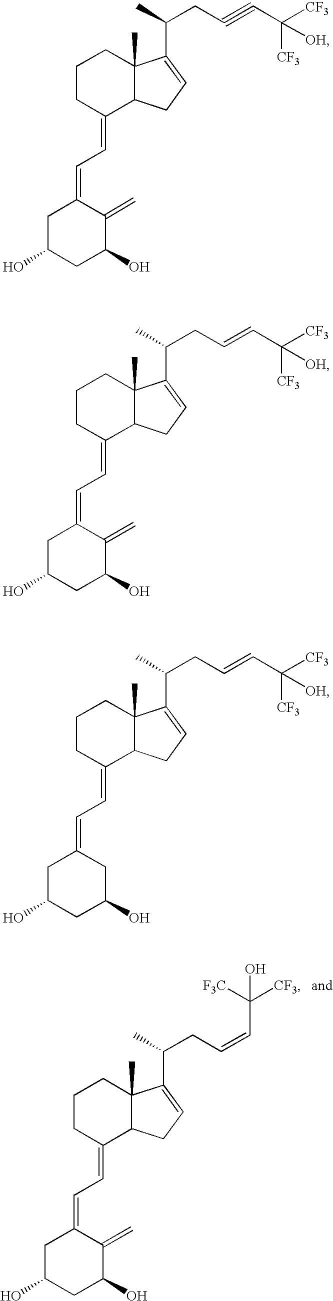

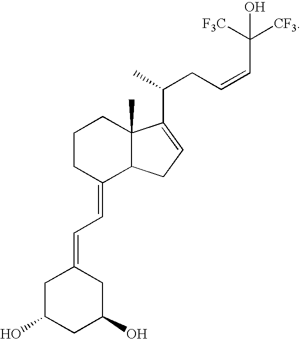

In other specific embodiments of the invention, the vitamin D compound is selected from the group consisting of:

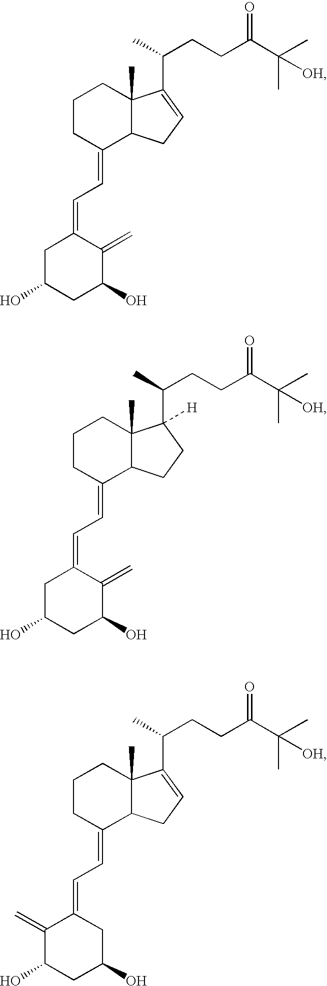

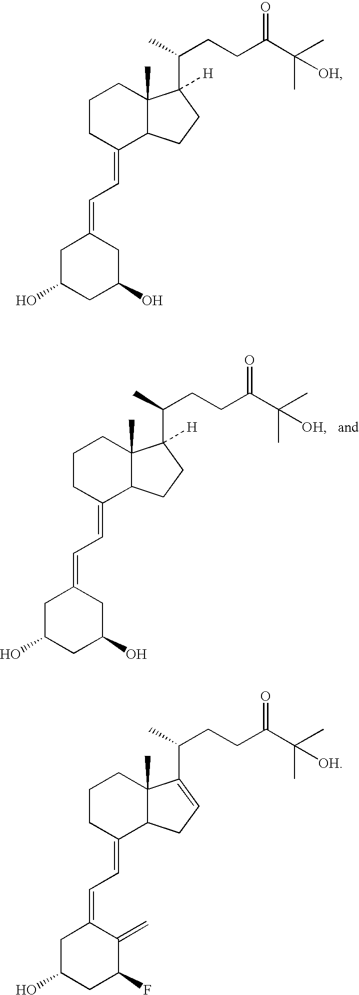

In yet another embodiment, the vitamin D compound is a “geminal” compound of formula (VI):

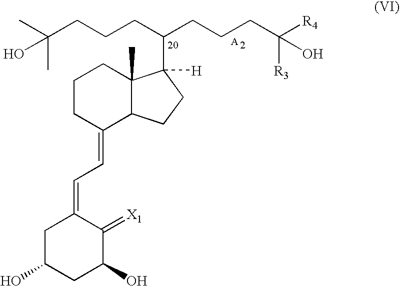

wherein:
X₁ is H₂ or CH₂;
A₂ is a single, a double or a triple bond;
R₃ is C₁-C₄ alkyl, hydroxyalkyl, or haloalkyl, e.g., fluoroalkyl, e.g., fluoromethyl and trifluoromethyl;
R₄ is C₁-C₄ alkyl, hydroxyalkyl or haloalkyl, e.g., fluoroalkyl, e.g., fluoromethyl and trifluoromethyl;
and the configuration at C₂₀ is R or S.

Compounds of this type may be referred to as “geminal” or “gemini” vitamin D₃ compounds due to the presence of two alkyl chains at C20.

An example geminal compound of formula (VI) is 1,25-dihydroxy-21-(3-hydroxy-3-methylbutyl)-19-nor-cholecalciferol (also referred to as Compound C herein):

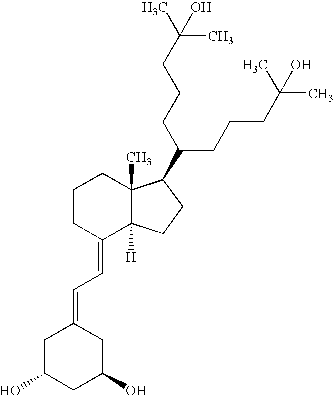

The synthesis of the above compound is described in WO98/49138 and U.S. Pat. No. 6,030,962 which are herein incorporated in their entirety by reference.

In further specific embodiments of the invention, the vitamin D compound is selected from the group of geminal compounds consisting of:

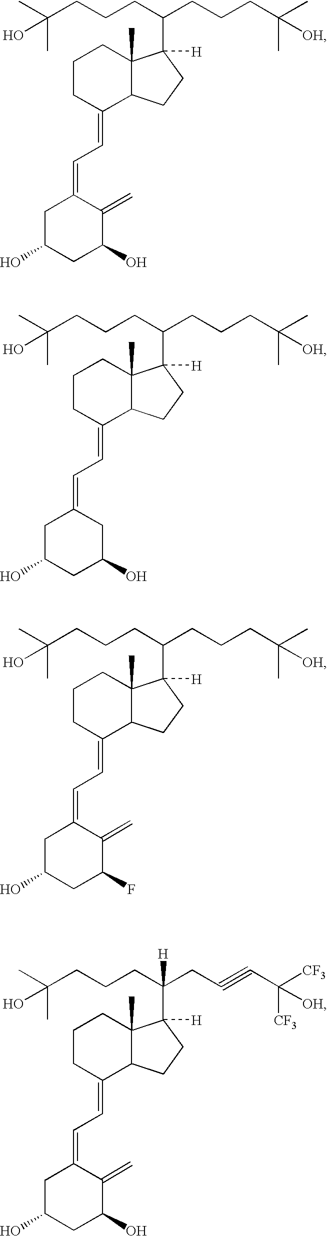

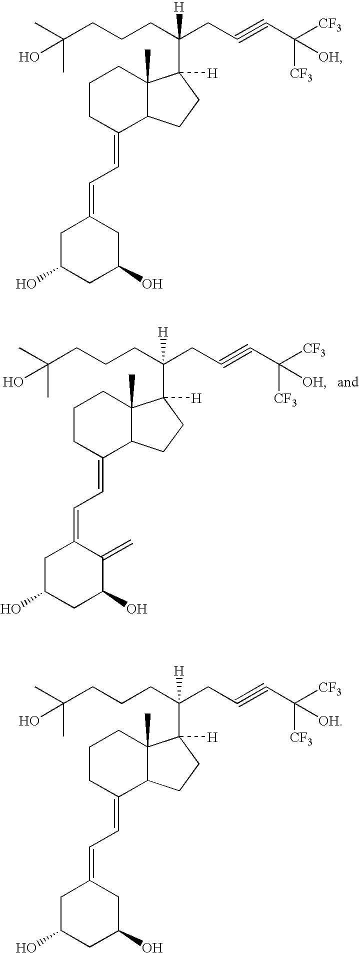

In yet another aspect, the invention provides gemini vitamin D₃ compounds of formula (IX):

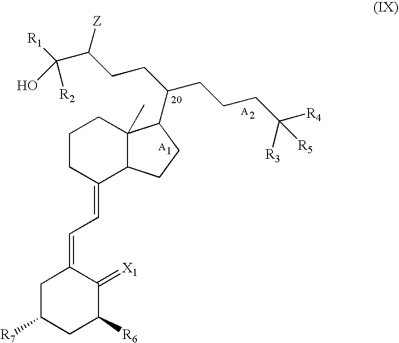

wherein:
A₁ is a single or double bond;
A₂ is a single, a double or a triple bond;
R₁, R₂, R₃ and R₄ are each independently C₁-C₄ alkyl, C₁-C₄ deuteroalkyl, hydroxyalkyl, or haloalkyl;
R₅, R₆ and R₇ are each independently hydroxyl, OC(O)C₁-C₄ alkyl, OC(O)hydroxyalkyl, or OC(O)haloalkyl;
the configuration at C20 is R or S;

### X₁ is H₂ or CH₂;

Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is C₁-C₄ deuteroalkyl; or Z is —OH, ═O, —SH, or —NH₂;
and pharmaceutically acceptable esters, salts, and prodrugs thereof.

Various embodiments of this aspect of the invention include individual compounds of formula I wherein: A₁ is a single bond; A₂ is a single bond; A₂ is a triple bond; R₁, R₂, R₃, and R₄ are each independently methyl or ethyl; R₁, R₂, R₃, and R₄ are each independently C₁-C₄ deuteroalkyl or haloalkyl; R₅ is hydroxyl; R₆ and R₇ are hydroxyl; R₆ and R₇ are each OC(O)C₁-C₄ alkyl; X₁ is H₂; X₁ is CH₂; Z is hydrogen; or Z is ═O.

In certain embodiments, R₅, R₆ and R₇ are hydroxyl. In other embodiments, R₆ and R₇ are each acetyloxy.

In yet other embodiments, Z is hydrogen when at least one of R₁ and R₂ is C₁-C₄ deuteroalkyl and at least one of R₃ and R₄ is haloalkyl or when at least one of R₁ and R₂ is haloalkyl and at least one of R₃ and R₄ is C₁-C₄ deuteroalkyl; Z is

—OH, ═O, —SH, or —NH₂ when X₁ is CH₂; Z is —OH, ═O, —SH, or —NH₂ when X₁ is H₂ and the configuration at C₂₀ is S; or Z is ═O, —SH, or —NH₂ when X₁ is H₂ and the configuration at C20 is R.

In one embodiment, Z is —OH.

Still other embodiments of this aspect of invention include those wherein X₁ is CH₂; A₂ is a single bond; R₁, R₂, R₃, and R₄ are each independently methyl or ethyl; and Z is —H. In one embodiment, X₁ is CH₂, A₂ is a single bond; R₁, R₂, R₃, and R₄ are each independently methyl or ethyl; and Z is ═O. In one embodiment, X₁ is H₂; A₂ is a single bond; R₁, R₂, R₃, and R₄ are each independently methyl or ethyl; the configuration at C₂₀ is S; and Z is —OH. In another embodiment, X₁ is H₂; A₂ is a single bond; R₁, R₂, R₃, and R₄ are each independently methyl or ethyl; and Z is ═O. In these embodiments, R₁, R₂, R₃, and R₄ are advantageously each methyl.

In certain embodiments, the haloalkyl is fluoroalkyl. Advantageously, fluoroalkyl is fluoromethyl or trifluoromethyl.

Additional embodiments of this aspect of the invention include compounds X₁ is H₂; A₂ is a triple bond; R₁ and R₂ are each C₁-C₄ deuteroalkyl; R₃ and R₄ are each haloalkyl; and Z is hydrogen.

In other embodiments, X₁ is CH₂; A₂ is a triple bond; R₁ and R₂ are each C₁-C₄ deuteroalkyl; R₃ and R₄ are each haloalkyl; and Z is hydrogen.

In these embodiments, R₁ and R₂ are advantageously each deuteromethyl and R₃ and R₄ are advantageously each trifluoromethyl.

In still further specific embodiments of the invention, the vitamin D compound is a geminal compound of formula (IX):

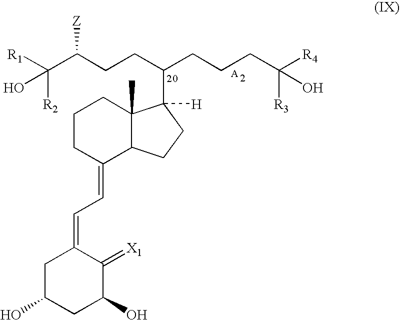

wherein:
X₁ is H₂ or CH₂;
A₂ is a single, a double or a triple bond;
R₁, R₂, R₃ and R₄ are each independently C₁-C₄ alkyl, hydroxyalkyl, or haloalkyl, e.g., fluoroalkyl, e.g., fluoromethyl and trifluoromethyl;
Z is —OH, Z may also be ═O, —NH₂ or —SH; and
the configuration at C₂₀ is R or S;
   and pharmaceutically acceptable esters, salts, and prodrugs thereof.

In a further embodiment, X₁ is CH₂. In another embodiment, A₂ is a single bond. In another, R₁, R₂, R₃, and R₄ are each independently methyl or ethyl. In a further embodiment, Z is —OH. In another, X₁ is CH₂; A₂ is a single bond; R₁, R₂, R₃, and R₄ are each independently methyl or ethyl; and Z is —OH. In an even further embodiment, R₁, R₂, R₃, and R₄ are each methyl.

In a further embodiment of the invention, the vitamin D compound is a geminal compound of the formula:

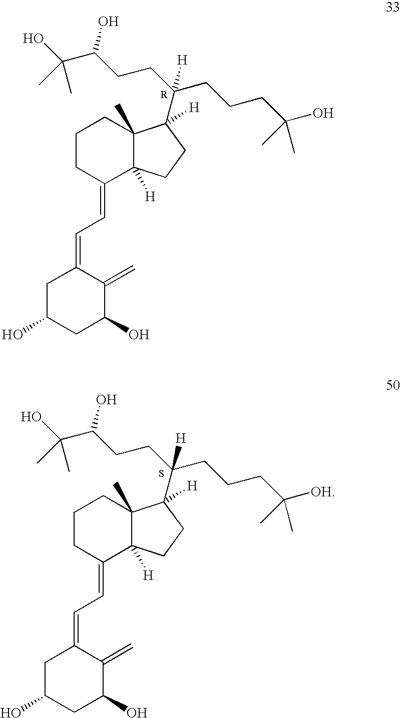

The chemical names of compounds 33 and 50 mentioned above are 1,25-dihydroxy-21-(2R,3-dihydroxy-3-methyl-butyl)-20R-cholecalciferol and 1,25-dihydroxy-21-(2R,3-dihydroxy-3-methyl-butyl)-20S-cholecalciferol respectively.

Additional embodiments of geminal compounds include the following vitamin D compounds for use in accordance with the invention:

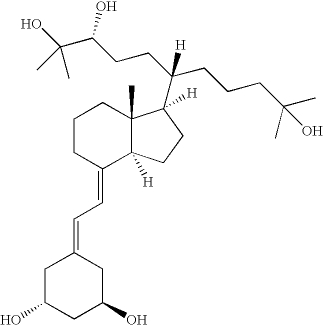

(1,25-Dihydroxy-21-(2R,3-dihydroxy-3-methyl-butyl)-20S-19-nor-cholecalciferol),

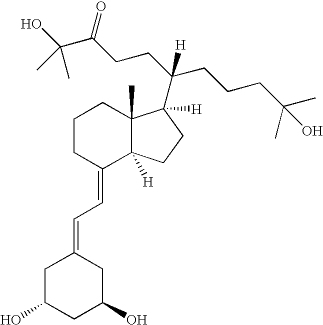

(1,25-Dihydroxy-20S-21-(3-hydroxy-3-methyl-butyl)-24-keto-19-nor-cholecalciferol),

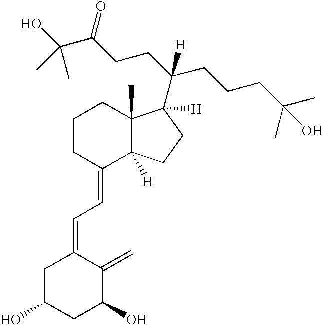

(1,25-Dihydroxy-20S-21-(3-hydroxy-3-methyl-butyl)-24-keto-cholecalciferol),

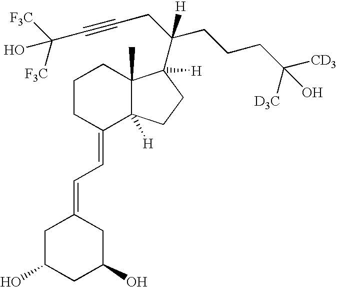

(1,25-Dihydroxy-21 (3-hydroxy-3-trifluoromethyl-4-trifluoro-butynyl)-26,27-hexadeutero-19-nor-20S-cholecalciferol) and

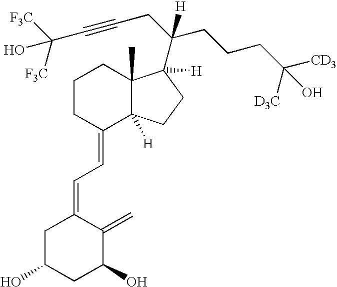

(1,25-Dihydroxy-21 (3-hydroxy-3-trifluoromethyl-4-trifluoro-butynyl)-26,27-hexadeutero-20S-cholecalciferol).

In further embodiments of the invention, the vitamin D compound is a compound of formula (X):

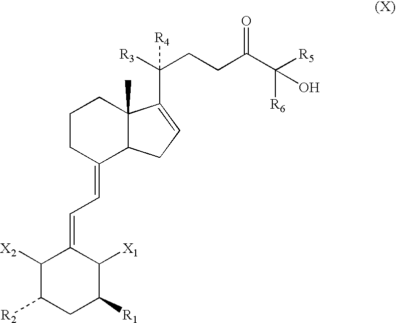

wherein:
X₁ and X₁ are each independently H₂ or ═CH₂, provided X₁ and X₁ are not both ═CH₂;
R₁ and R₂ are each independently, hydroxyl, OC(O)C₁-C₄ alkyl, OC(O)hydroxyalkyl, OC(O)fluororalkyl;
R₃ and R₄ are each independently hydrogen, C₁-C₄ alkyl hydroxyalkyl or haloalkyl, or R₃ and R₄ taken together with C₂₀ form C₃-C₆ cylcoalkyl; and
R₅ and R₆ are each independently C₁-C₄ alkyl
   and pharmaceutically acceptable esters, salts, and prodrugs thereof.

Suitably R₃ and R₄ are each independently hydrogen, C₁-C₄ alkyl, or R₃ and R₄ taken together with C₂₀ form C₃-C₆ cylcoalkyl.

In one example set of compounds R₅ and R₆ are each independently C₁-C₄ alkyl.

In another example set of compounds R₅ and R₆ are each independently haloalkyl e.g., C₁-C₄ fluoroalkyl.

When R₃ and R₄ are taken together with C20 to form C₃-C₆ cycloalkyl, an example is cyclopropyl.

In one embodiment, X₁ and X₁ are each H₂. In another embodiment, R₃ is hydrogen and R₄ is C₁-C₄ alkyl. In a preferred embodiment R₄ is methyl.

In another embodiment, R₅ and R₆ are each independently methyl, ethyl, fluoromethyl or trifluoromethyl. In a preferred embodiment, R₅ and R₆ are each methyl.

In yet another embodiment, R₁ and R₁ are each independently hydroxyl or OC(O)C₁-C₄ alkyl. In a preferred embodiment, R₁ and R₁ are each OC(O)C₁-C₄ alkyl. In another preferred embodiment, R₁ and R₁ are each acetyloxy.

An example of such a compound is 1,3-O-diacetyl-1,25-dihydroxy-16-ene-24-keto-19-nor-cholecalciferol, having the following structure:

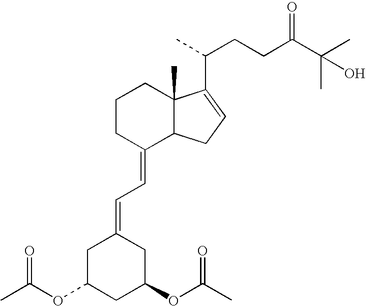

In another embodiment of the invention the vitamin D compound for use in accordance with the invention is 2-methylene-19-nor-20(S)-1-alpha,25-hydroxyvitamin D₃:

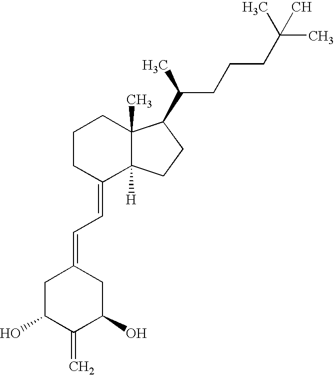

The synthesis of this and related compounds is described in WO02/05823 and U.S. Pat. No. 5,536,713 which are herein incorporated in their entirety by reference.

In another embodiment of the invention, the vitamin D compound is a compound of the formula (XII):

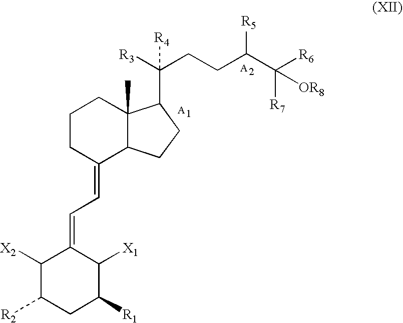

wherein:
A₁ is single or double bond;
A₂ is a single, double or triple bond;
X₁ and X₂ are each independently H or ═CH₂, provided X₁ and X₂ are not both ═CH₂;
R₁ and R₂ are each independently OC(O)C₁-C₄ alkyl (for example OAc), OC(O)hydroxyalkyl or OC(O)haloalkyl, such as OC(O)C₁-C₄ alkyl or OC(O)hydroxyalkyl;
R₁ and/or R₂ can alternatively be OH;
R₃, R₄ and R₅ are each independently hydrogen, C₁-C₄ alkyl, hydroxyalkyl, or haloalkyl, or R₃ and R₄ taken together with C₂₀ form C₃-C₆ cycloalkyl; and
R₆ and R₇ are each independently C₁₋₄alkyl or haloalkyl; and
R₈ is H, —COC₁-C₄alkyl (e.g. Ac), —COhydroxyalkyl or —COhaloalkyl; and
   pharmaceutically acceptable esters, salts, and prodrugs thereof.

When R₃ and R₄ are taken together with C₂₀ to form C₃-C₆ cycloalkyl an example is cyclopropyl.

Suitably R₆ and R₇ are each independently haloalkyl. R₆ may suitably represent H or Ac.

In one embodiment, A₁ is a single bond and A₂ is a single bond, E or Z double bond, or a triple bond. In another embodiment, A₁ is a double bond and A₂ is a single bond, E or Z double bond, or a triple bond. One of ordinary skill in the art will readily appreciate that when A₂ is a triple bond, R₅ is absent

In one embodiment, X₁ and X₂ are each H. In another embodiment, X₁ is CH₂ and X₂ is H₂. In another embodiment, R₃ is hydrogen and R₄ is C₁-C₄ alkyl. In a preferred embodiment R₄ is methyl.

In another example set of compounds R₁ and R₂ both represent OAc.

In one set of example compounds R₆ and R₇ are each independently C₁₋₄alkyl. In another set of example compounds R₆ and R₇ are each independently haloalkyl. In another embodiment, R₆ and R₇ are each independently methyl, ethyl or fluoroalkyl. In a preferred embodiment, R₆ and R₈ are each trifluoroalkyl, e.g., trifluoromethyl.

Suitably R₅ represents hydrogen.

Thus, in certain embodiments, vitamin D compounds for use in accordance with the invention are represented by formula (XII):

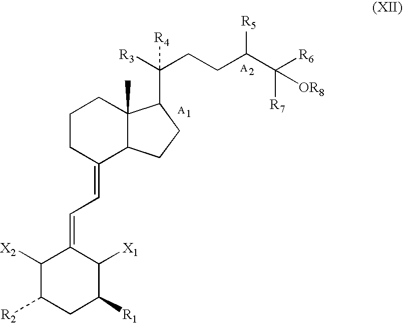

wherein:
A₁ is single or double bond;
A₂ is a single, double or triple bond;
X₁ and X₂ are each independently H or ═CH₂, provided X₁ and X₂ are not both ═CH₂;
R₁ and R₂ are each independently OC(O)C₁-C₄ alkyl, OC(O)hydroxyalkyl, or OC(O)haloalkyl;
R₃, R₄ and R₅ are each independently hydrogen, C₁-C₄ alkyl, hydroxyalkyl, or haloalkyl, or R₃ and R₄ taken together with C₂₀ form C₃-C₆ cycloalkyl;
R₆ and R₇ are each independently haloalkyl; R₆ and R₇ can alternatively be alkyl; and
R₈ is H, C(O)C₁-C₄ alkyl, C(O)hydroxyalkyl, or C(O)haloalkyl; and
   pharmaceutically acceptable esters, salts, and prodrugs thereof. In preferred embodiments, when A₁ is a single bond, R₃ is hydrogen and R₄ is methyl, then A₂ is a double or triple bond.

An example compound of the above-described formula (XII) which is particularly preferred in the context of the present invention is 1,3-di-O-acetyl-1,25-dihydroxy-16,23Z-diene-26,27-hexafluoro-19-nor-cholecalciferol:

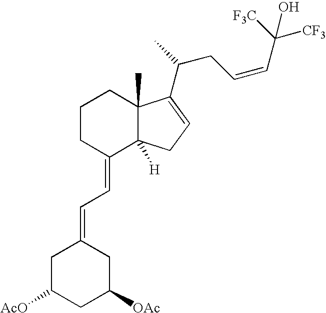

In another preferred embodiment the compound is one of formula (XIII), wherein R₁ and R₂ are each OAc; A₁ is a double bond; A₂ is a triple bond; and R₈ is either H or Ac:

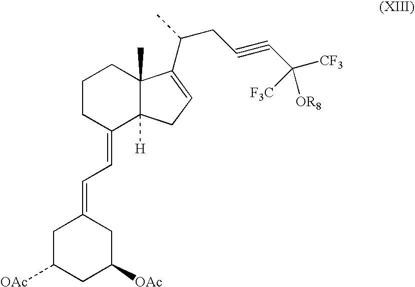

In certain embodiments of the above-represented formula (XII), vitamin D compounds for use in accordance with the invention are represented by the formula (XIV):

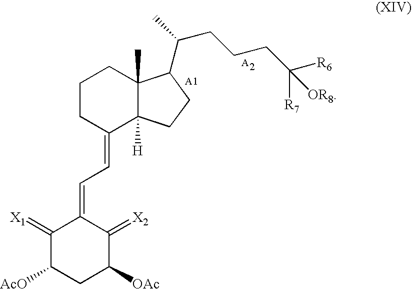

In a preferred embodiment, X₁ is ═CH₂ and X₂ is H₂. When A₁ is a single bond, and A₂ is a triple bond, it is preferred that R₈ is H or C(O)CH₃, and R₆ and R₇ are alkyl, preferably methyl. When A₁ is a single bond, and A₂ is a single bond, it is preferred that R₈ is H or C(O)CH₃, and R₆ and R₇ are alkyl, preferably methyl. When A₁ is a double bond, and A₂ is a single bond, it is preferable that R₈ is H or C(O)CH₃, and R₆ and R₇ are alkyl, preferably methyl.

In another preferred embodiment, X₁ and X₂ are each H₂. When A₁ is a single bond, and A₂ is a triple bond, it is preferred that R₈ is H or C(O)CH₃, and R₆ and R₇ are alkyl or haloalkyl. It is preferred that the alkyl group is methyl, and the haloalkyl group is trifluoroalkyl, preferably trifluoromethyl. When A₁ is a single bond, and A₂ is a double bond, it is preferred that R₈ is H or C(O)CH₃, R₆ and R₇ are haloalkyl, preferably trifluoroalkyl, preferably trifluoromethyl. When A₁ is a double bond, and A₂ is a single bond, it is preferred that R₈ is H or C(O)CH₃, R₆ and R₇ are alkyl, preferably methyl.

Other example compounds of the above-described formula (XIV) include:
1,3-di-O-acetyl-1,25-dihydroxy-23-yne-cholecalciferol;
1,3-di-O-acetyl-1,25-dihydroxy-16-ene-23-yne-cholecalciferol;
1,3-di-O-acetyl-1,25-dihydroxy-16,23E-diene-cholecalciferol;
1,3-di-O-acetyl-1,25-dihydroxy-16-ene-cholecalciferol;
1,3,25-Tri-O-acetyl-1,25-dihydroxy-16-ene-23-yne-26,27-hexafluoro-holecalciferol:
1,3-di-O-acetyl-1,25-dihydroxy-16-ene-23-yne-26,27-hexafluoro-cholecalciferol;
1,3-Di-O-acetyl-1,25-dihydroxy-16,23E-diene-25R-26-trifluoro-cholecalciferol;
1,3-Di-O-acetyl-1,25-Dihydroxy-16-ene-23-yne-26,27-hexafluoro-19-nor-cholecalciferol;
1,3,25-Tri-O-acetyl-1,25-Dihydroxy-16-ene-23-yne-26,27-hexafluoro-19-nor-cholecalciferol;
1,3-di-O-acetyl-1,25-dihydroxy-16-ene-19-nor-cholecalciferol;
1,3-Di-O-acetyl-1,25-dihydroxy-16-ene-23-yne-19-nor-cholecalciferol;
1,3-Di-O-acetyl-1,25-dihydroxy-16-ene-23-yne-26,27-bishomo-19-nor-cholecalciferol.

In certain other embodiments of the above-represented formula (XII), the vitamin D compounds for use in accordance with the invention are represented by the formula (XV):

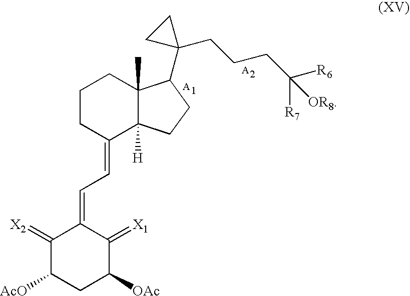

Other example compounds of the above-described formula (XV) include:
1,3-di-O-acetyl-1,25-dihydroxy-20-cyclopropyl-23-yne-19-nor-cholecalciferol:
1,3,25-tri-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-cholecalciferol;
1,3-di-O-acetyl-1,25-dihydroxy-20-cyclopropyl-23Z-ene-26,27-hexafluoro-19-nor-cholecalciferol;
1,3-di-O-acetyl-1,25-dihydroxy-20-cyclopropyl-cholecalciferol;
1,3-di-O-acetyl-1,25-dihydroxy-16-ene-20-cyclopropyl-19-nor-cholecalciferol; and
1,3-di-O-acetyl-1,25-dihydroxy-16-ene-20-cyclopropyl-cholecalciferol.

A preferred compound of formula (XV) is 1,3-di-O-acetyl-1,25-dihydroxy-20-cyclopropyl-23E-ene-26,27-hexafluoro-19-nor-cholecalciferol:

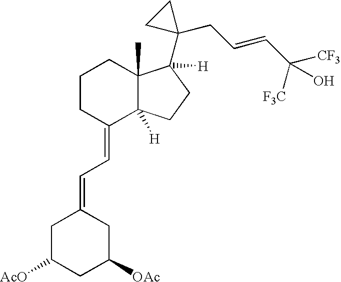

An example of another preferred compound is 1,3-Di-O-acetyl-1,25-dihydroxy-20-cyclopropyl-cholecalciferol (referred to as “Compound D”) having the formula:

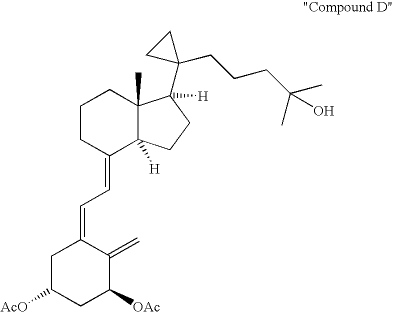

Such compounds are described in WO2005/030222, the contents of which are herein incorporated by reference in their entirety. The invention also embraces use of esters and salts of Compound D. Esters include pharmaceutically acceptable labile esters that may be hydrolysed in the body to release Compound D. Salts of Compound D include adducts and complexes that may be formed with alkali and alkaline earth metal ions and metal ion salts such as sodium, potassium and calcium ions and salts thereof such as calcium chloride, calcium malonate and the like. However, although Compound D may be administered as a pharmaceutically acceptable salt or ester thereof, preferably Compound D is employed as is i.e., it is not employed as an ester or a salt thereof.

Another compound is 1,25-dihydroxy-20,21,28-cyclopropyl-cholecalciferol having the formula:

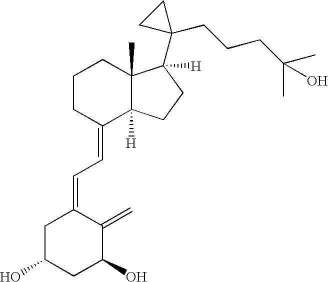

The compound is described in U.S. Pat. No. 6,492,353, the contents of which are herein incorporated by reference in their entirety.

The invention also embraces use of esters and salts of 1,25-dihydroxy-20,21,28-cyclopropyl-cholecalciferol. Esters include pharmaceutically acceptable labile esters that may be hydrolysed in the body to release 1,25-dihydroxy-20,21,28-cyclopropyl-cholecalciferol. Salts of 1,25-dihydroxy-20,21,28-cyclopropyl-cholecalciferol include adducts and complexes that may be formed with alkali and alkaline earth metal ions and metal ion salts such as sodium, potassium and calcium ions and salts thereof such as calcium chloride, calcium malonate and the like. However, although 1,25-dihydroxy-20,21,28-cyclopropyl-cholecalciferol may be administered as a pharmaceutically acceptable salt or ester thereof, preferably it is employed as is i.e., it is not employed as an ester or a salt thereof.

Other preferred vitamin D compounds for use in accordance with the invention included those having formula (XVII):

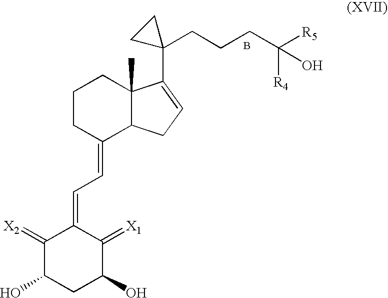

wherein:
B is single, double, or triple bond;
X₁ and X₂ are each independently H₂ or CH₂, provided X₁ and X₂ are not both CH₂; and
R₄ and R₅ are each independently alkyl or haloalkyl.

Compounds of formula (XVII) including the following:
1,25-Dihydroxy-16-ene-23-yne-20-cyclopyl-cholecalciferol:

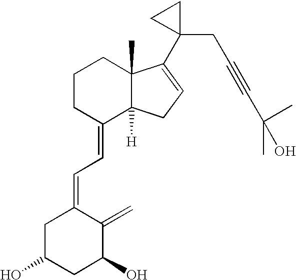

1,25-Dihydroxy-16-ene-23-yne-20-cyclopropyl-19-nor-cholecalciferol:

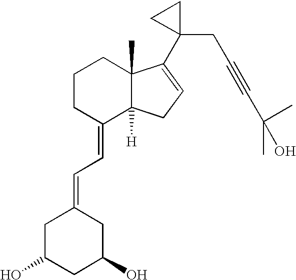

1,25-Dihydroxy-16-ene-20-cylopropyl-23-yne-26,27-hexafluoro-19-nor-cholecalciferol:

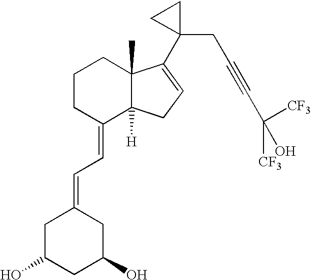

1,25-Dihydroxy-16-ene-20-cyclopropyl-23-yne-26,27-hexafluoro-holecalciferol:

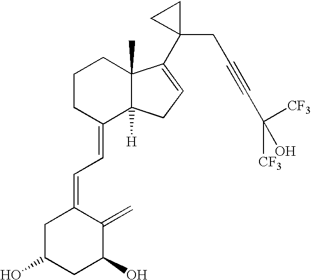

1,25-Dihydroxy-16,23E-diene-20-cyclopropyl-26,27-hexafluoro-19-nor-cholecalciferol:

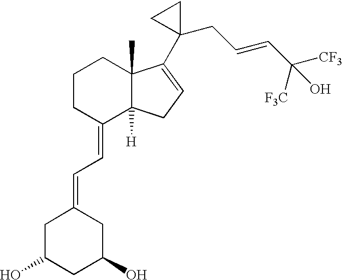

1,25-Dihydroxy-16,23E-diene-20-cyclopropyl-26,27-hexafluoro-cholecalciferol:

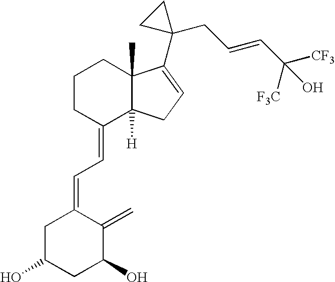

1,25-Dihydroxy-16,23Z-diene-20-cyclopropyl-26,27-hexafluoro-19-nor-holecalciferol:

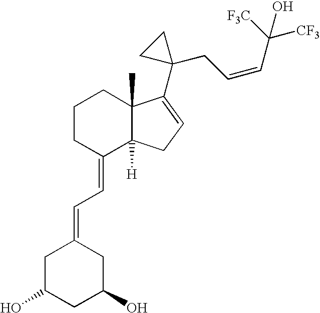

1,25-Dihydroxy-16,23Z-diene-20-cyclopropyl-26,27-hexafluoro-holecalciferol:

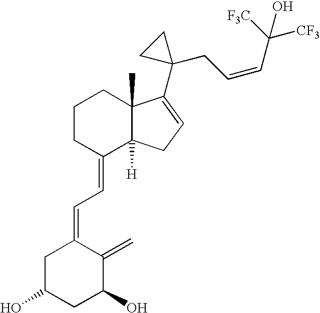

1,25-Dihydroxy-16-ene-20-cyclopropyl-19-nor-cholecalciferol:

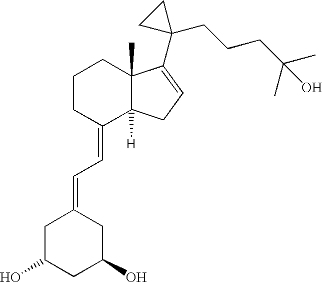

1,25-Dihydroxy-16-ene-20-cyclopropyl-cholecalciferol:

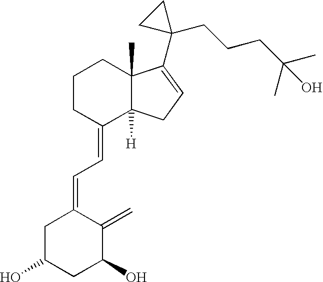

In a further embodiment, vitamin D compounds for use in the invention are compounds of the formula (XVI):

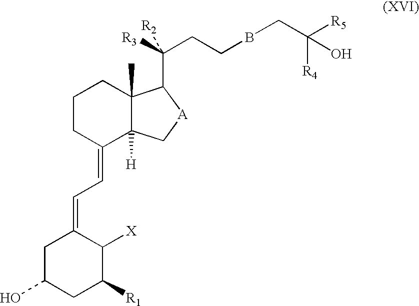

wherein:
X is H₂ or CH₂
R₁ is hydrogen, hydroxy or fluorine R₂ is hydrogen or methyl
R₃ is hydrogen or methyl. When R₂ or R₃ is methyl, R₃ or R₂ must be hydrogen.
R₄ is methyl, ethyl or trifluoromethyl
R₅ is methyl, ethyl or trifluoromethyl
A is a single or double bond
B is a single, E-double, Z-double or triple bond.

In preferred compounds, each of R₄ and R₅ is methyl or ethyl, for example 1-alpha-fluoro-25-hydroxy-16,23E-diene-26,27-bishomo-20-epi-cholecalciferol (referred to as “Compound A” in examples, having the formula:

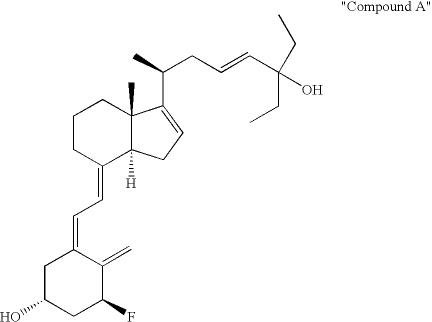

Such compounds are described in U.S. Pat. No. 5,939,408 and EP808833, the contents of which are herein incorporated by reference in their entirety. The invention also embraces use of esters and salts of Compound A. Esters include pharmaceutically acceptable labile esters that may be hydrolysed in the body to release Compound A. Salts of Compound A include adducts and complexes that may be formed with alkali and alkaline earth metal ions and metal ion salts such as sodium, potassium and calcium ions and salts thereof such as calcium chloride, calcium malonate and the like. However, although Compound A may be administered as a pharmaceutically acceptable salt or ester thereof, preferably Compound A is employed as is i.e., it is not employed as an ester or a salt thereof.

Another vitamin D compound of the invention is 1,25-dihydroxy-21(3-hydroxy-3-trifluoromethyl-4-trifluoro-butynyl)-26,27-hexadeutero-19-nor-20S-cholecalciferol.

Still other preferred vitamin D compounds for use in accordance with the invention include those having formula (XVIII):

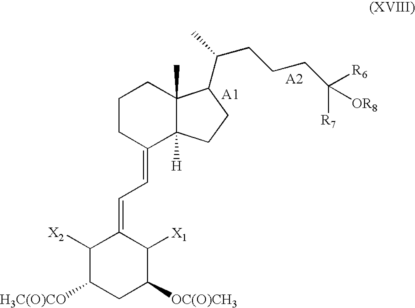

In one embodiment, A₁ is a double bond, and X₁ is ═CH₂ and X₂ is H₂. When A₂ is a triple bond, it is preferred that R₈ is H or C(O)CH₃, and R₆ and R₇ are alkyl or haloalkyl. It is preferred that the alkyl group is methyl and the haloalkyl group is trifluoroalkyl, preferably trifluoromethyl. When A₂ is a double bond, it is preferred that R₈ is H or C(O)CH₃, and R₆ and R₇ are alkyl, preferably methyl. It is also preferred that R₆ and R₇ are independently alkyl and haloalkyl. When A₂ is a single bond, it is preferred that R₈ is H or C(O)CH₃, and R₆ and R₇ are alkyl, preferably methyl.

In a preferred embodiment, A₁ is a double bond, and X₁ and X₂ are each H₂. When A₂ is a triple bond, it is preferred that R₈ is H or C(O)CH₃, and R₆ and R₇ are alkyl or haloalkyl. It is preferred that the alkyl group is methyl or ethyl and the haloalkyl group is trifluoroalkyl, preferably trifluoromethyl. When A₂ is a double bond, it is preferred that R₅ is H or C(O)CH₃, and R₆ and R₇ are haloalkyl, preferably trifluoroalkyl, preferably trifluoromethyl. When A₂ is a single bond, it is preferred that R₈ is H or C(O)CH₃, and R₆ and R₇ are alkyl, preferably methyl.

In another embodiment of the invention of formula (XVIII), R₁ and R₂ are OC(O)CH₃, A₁ is a single bond, and A₂ is a single, double or triple bond, except that when R₃ is H and R₄ is methyl, A₂ is a double or triple bond. In a preferred embodiment, R₃ is H; R₄ is methyl, R₅ is absent, R₈ is H or C(O)CH₃, and R₆ and R₇ are alkyl, preferably methyl.

Preferred compounds of the present include the following: 1,3-Di-O-acetyl-1,25-dihydroxy-16,23Z-diene-26,27-hexafluoro-19-nor-cholecalciferol, 1,3-Di-O-acetyl-1,25-Dihydroxy-16-ene-23-yne-26,27-hexafluoro-19-nor-cholecalciferol, 1,3,25-Tri-O-acetyl-1,25-Dihydroxy-16-ene-23-yne-26,27-hexafluoro-19-nor-cholecalciferol, 1,3-Di-O-acetyl-1,25-dihydroxy-16-ene-23-yne-cholecalciferol, 1,3-Di-O-acetyl-1,25-dihydroxy-16,23E-diene-cholecalciferol, 1,3-Di-O-acetyl-1,25-dihydroxy-16-ene-cholecalciferol, 1,3,25-Tri-O-acetyl-1,25-dihydroxy-16-ene-23-yne-26,27-hexafluoro-cholecalciferol, 1,3-Di-O-acetyl-1,25-dihydroxy-16-ene-23-yne-26,27-hexafluoro-cholecalciferol, 1,3-Di-O-acetyl-1,25-dihydroxy-16,23E-diene-25R,26-trifluoro-cholecalciferol, 1,3-Di-O-acetyl-1,25-dihydroxy-16-ene-19-nor-cholecalciferol, 1,3-Di-O-Acetyl-1,25-dihydroxy-16-ene-23-yne-19-nor-cholecalciferol, 1,3-Di-O-acetyl-1,25-dihydroxy-16-ene-23-yne-26,27-bishomo-19-nor-holecalciferol and 1,3-Di-O-acetyl-1,25-dihydroxy-23-yne-cholecalciferol. These compounds can be prepared, e.g., as described in PCT Publication WO2005030222. The use of compounds having the structures given above is extended to pharmaceutically acceptable esters, salts, and prodrugs thereof.

Yet further preferred vitamin D compounds for use in accordance with the invention include those having formula (XIX):

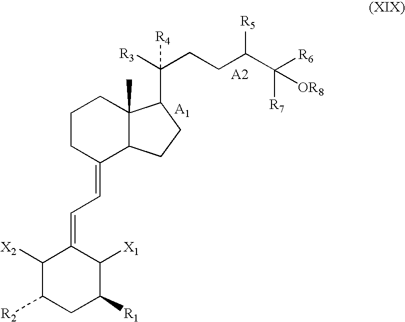

wherein:
A₁ is single or double bond;
A₂ is a single, double or triple bond,
X₁ and X₂ are each independently H₂ or CH₂, provided X₁ and X₂ are not both CH₂;
R₁ and R₂ are each independently OC(O)C₁-C₄ alkyl, OC(O)hydroxyalkyl, or OC(O)haloalkyl;
R₃, R₄ and R₅ are each independently hydrogen, C₁-C₄ alkyl, hydroxyalkyl, or haloalkyl, or R₃ and R₄ taken together with C₂₀ form C₃-C₆ cylcoalkyl;
R₆ and R₇ are each independently haloalkyl; and
R₈ is H, OC(O)C₁-C₄ alkyl, OC(O)hydroxyalkyl, or OC(O)haloalkyl; and
pharmaceutically acceptable esters, salts, and prodrugs thereof. In preferred embodiments, R₆ and R₇ are each independently trihaloalkyl, especially trifluoromethyl.

These compounds can be prepared, e.g., as described in PCT Publication WO2005030222, the contents of which are incorporated herein by reference. The use of compounds having the structures given above is extended to pharmaceutically acceptable esters, salts, and prodrugs thereof.

A vitamin D compound of particular interest is calcitriol (also referred to as Compound B herein.

The use of compounds having the structures given above is extended to pharmaceutically acceptable esters, salts, and prodrugs thereof.

Other example compounds of use in the invention which are vitamin D receptor agonists include paricalcitol (ZEMPLAR™) (see U.S. Pat. No. 5,587,497), tacalcitol (BONALFA™) (see U.S. Pat. No. 4,022,891), doxercalciferol (HECTOROL™) (see Lam et al. (1974) Science 186, 1038), maxacalcitol (OXAROL™) (see U.S. Pat. No. 4,891,364), calcipotriol (DAIVONEX™) (see U.S. Pat. No. 4,866,048), and falecalcitriol (FULSTAN™).

Other compounds include ecalcidene, calcithiazol and tisocalcitate.

Other compounds include atocalcitol, lexacalcitol and seocalcitol.

Another compound of possible interest is secalciferol (“OSTEO D”).

Other non-limiting examples of vitamin D compounds that may be of use in accordance with the invention include those described in published international applications: WO 01/40177, WO0010548, WO0061776, WO0064869, WO0064870, WO0066548, WO0104089, WO0116099, WO0130751, WO0140177, WO0151464, WO0156982, WO0162723, WO0174765, WO0174766, WO0179166, WO0190061, WO0192221, WO0196293, WO02066424, WO0212182, WO0214268, WO03004036, WO03027065, WO03055854, WO03088977, WO04037781, WO04067504, WO8000339, WO8500819, WO8505622, WO8602078, WO8604333, WO8700834, WO8910351, WO9009991, WO9009992, WO9010620, WO9100271, WO9100855, WO9109841, WO9112239, WO9112240, WO9115475, WO9203414, WO9309093, WO9319044, WO9401398, WO9407851, WO9407852, WO9408958, WO9410139, WO9414766, WO9502577, WO9503273, WO9512575, WO9527697, WO9616035, WO9616036, WO9622973, WO9711053, WO9720811, WO9737972, WO9746522, WO9818759, WO9824762, WO9828266, WO9841500, WO9841501, WO9849138, WO9851663, WO9851664, WO9851678, WO9903829, WO9912894, WO9915499, WO9918070, WO9943645, WO9952863, those described in U.S. Pat. No. 3,856,780, U.S. Pat. No. 3,994,878, U.S. Pat. No. 4,021,423, U.S. Pat. No. 4,026,882, U.S. Pat. No. 4,028,349, U.S. Pat. No. 4,225,525, U.S. Pat. No. 4,613,594, U.S. Pat. No. 4,804,502, U.S. Pat. No. 4,898,855, U.S. Pat. No. 5,039,671, U.S. Pat. No. 5,087,619, U.S. Pat. No. 5,145,846, U.S. Pat. No. 5,247,123, U.S. Pat. No. 5,342,833, U.S. Pat. No. 5,428,029, U.S. Pat. No. 5,451,574, U.S. Pat. No. 5,612,328, U.S. Pat. No. 5,747,479, U.S. Pat. No. 5,804,574, U.S. Pat. No. 5,811,414, U.S. Pat. No. 5,856,317, U.S. Pat. No. 5,872,113, U.S. Pat. No. 5,888,994, U.S. Pat. No. 5,939,408, U.S. Pat. No. 5,962,707, U.S. Pat. No. 5,981,780, US6017908, U.S. Pat. No. 6,030,962, U.S. Pat. No. 6,040,461, U.S. Pat. No. 6,100,294, U.S. Pat. No. 6,121,312, U.S. Pat. No. 6,329,538, U.S. Pat. No. 6,331,642, U.S. Pat. No. 6,392,071, U.S. Pat. No. 6,452,028, U.S. Pat. No. 6,479,538, U.S. Pat. No. 6,492,353, U.S. Pat. No. 6,537,981, U.S. Pat. No. 6,544,969, U.S. Pat. No. 6,559,138, U.S. Pat. No. 6,667,298, U.S. Pat. No. 6,683,219, U.S. Pat. No. 6,696,431, U.S. Pat. No. 6,774,251, and those described in published US patent applications: US2001007907, US2003083319, US2003125309, US2003130241, US2003171605, US2004167105. Additional vitamin D compounds of use in accordance with the present invention include those described in U.S. Pat. No. 4,929,609, U.S. Pat. No. 5,393,900, U.S. Pat. No. 5,747,478, WO2005/082375, WO2005/030223, WO2005/030222, WO2005/027923, WO2004/098522 and WO2004/098507.

It will be noted that the structures of some of the compounds of the invention include asymmetric carbon atoms. Accordingly, it is to be understood that the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

The invention also provides a pharmaceutical composition, comprising an effective amount of a vitamin D compound as described herein and a pharmaceutically acceptable carrier. In a further embodiment, the effective amount is effective to treat endometriosis, as described previously.

In an embodiment, the vitamin D compound is administered to the subject using a pharmaceutically-acceptable formulation, e.g., a pharmaceutically-acceptable formulation that provides sustained delivery of the vitamin D compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension, (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those vitamin D compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a vitamin D compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a vitamin D compound(s) with the carrier and, optionally, one or more accessory ingredients. In general, the formulations are prepared by uniformly and intimately bringing into association a vitamin D compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the vitamin D compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more vitamin D compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a vitamin D compound(s) include powders, sprays, ointments, pastes, creams, lotions, gels, solutions, patches and inhalants. The active vitamin D compound(s) may be mixed under sterile conditions with a pharmaceutically-acceptable carrier, and with any preservatives, buffers, or propellants which may be required.

The ointments, pastes, creams and gels may contain, in addition to vitamin D compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D compound(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The vitamin D compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Pharmaceutical compositions of the invention suitable for parenteral administration comprise one or more vitamin D compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of vitamin D compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the vitamin D compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the vitamin D compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 300 μg per day

A preferred dose of the vitamin D compound for the present invention is the maximum that a patient can tolerate and not develop hypercalcemia. Preferably, the vitamin D compound of the present invention is administered at a concentration of about 0.001 ug to about 100 ug per kilogram of body weight, about 0.001-about 10 ug/kg or about 0.001 ug-about 100 ug/kg of body weight. Ranges intermediate to the above-recited values are also intended to be part of the invention.

The vitamin D compound may be administered separately, sequentially or simultaneously in separate or combined pharmaceutical formulations with a second medicament for the treatment of endometriosis.

### SYNTHESIS OF COMPOUNDS OF THE INVENTION

A number of the compounds of the present invention can be prepared by incubation of vitamin D₃ analogues in cells, for example, incubation of vitamin D₃ analogues in either UMR 106 cells or Ros 17/2.8 cells results in production of vitamin D₃ compounds of the invention. For example, Incubation of 1,25-dihydroxy-16-ene-5,6-trans-calcitriol in UMR 106 cells results in production of the 1,25-dihydroxy-16-ene-24-oxo-5,6-trans-calcitriol.

In addition to the methods described herein, compounds of the present invention can be prepared using a variety of synthetic methods. For example, one skilled in the art would be able to use methods for synthesizing existing vitamin D₃ compounds to prepare compounds of the invention (see e.g., Bouillon, R. et al., (1995) Endocrine Reviews 16(2):201-204; Ikekawa N. (1987) Med. Res. Rev. 7:333-366; DeLuca H. F. and Ostrem V. K. (1988) Prog. Clin. Biol. Res. 259:41-55; Ikekawa N. and Ishizuka S. (1992) CRC Press 8:293-316; Calverley M. J. and Jones G. (1992) Academic Press 193-270; Pardo R. and Santelli M. (1985) Bull. Soc. Chim. Fr:98-114; Bythgoe B. (1980) Chem. Soc. Rev. 449-475; Quinkert G. (1985) Synform 3:41-122; Quinkert G. (1986) Synform 4:131-256; Quinkert G. (1987) Synform 5:1-85; Mathieu C. et al. (1994) Diabetologia 37:552-558; Dai H. and Posner G. H. (1994) Synthesis 1383-1398); DeLuca et al., WO 97/11053.

Exemplary methods of synthesis include the photochemical ring opening of a 1-hydroxylated side chain-modified derivative of 7-dehydrocholesterol which initially produces a previtamin that is easily thermolyzed to vitamin D₃ in a well known fashion (Barton D. H. R. et al. (1973) J. Am. Chem. Soc. 95:2748-2749; Barton D. H. R. (1974) JCS Chem. Comm. 203-204); phosphine oxide coupling method developed by (Lythgoe et al (1978) JCS Perkin Trans. 1:590-595) which comprises coupling a phosphine oxide to a Grundmann's ketone derivative to directly produce a 1-alpha,25(OH)₂D₃ skeleton as described in Baggiolini E. G., et al. (1986) J. Org. Chem. 51:3098-3108; DeSchrijver J. and DeClercq P. J. (1993) Tetrahed Lett 34:4369-4372; Posner G. H and Kinter C. M. (1990) J. Org. Chem. 55:3967-3969; semihydrogenation of dienynes to a previtamin structure that undergoes rearrangement to the corresponding vitamin D₃ analogue as described by Harrison R. G. et al. (1974) JCS Perkin Trans. 1:2654-2657; Castedo L. et al. (1988) Tetrahed Lett 29:1203-1206; Mascarenas J. S. (1991) Tetrahedron 47:3485-3498; Barrack S. A. et al. (1988) J. Org. Chem. 53:1790-1796) and Okamura W. H. et al. (1989) J. Org. Chem. 54:4072-4083; the vinylallene approach involving intermediates that are subsequently arranged using heat or a combination of metal catalyzed isomerization followed by sensitized photoisomerization (Okamura W. H. et al. (1989) J. Org. Chem. 54:4072-4083; Van Alstyne E. M. et al. (1994) J. Am. Chem. Soc. 116:6207-6210); the method described by Trost et al. B. M. et al. J. Am. Chem. Soc. 114:9836-9845; Nagasawa K. et al. (1991) Tetrahed Lett 32:4937-4940 involves an acyclic A-ring precursor which is intramolecular cross-coupled to the bromoenyne leading directly to the formation of 1,25(OH)₂D₃ skeleton; a tosylated derivative which is isomerized to the i-steroid that can be modified at carbon-1 and then subsequently back-isomerized under sovolytic conditions to form 1-alpha,25(OH)₂D₂ or analogues thereof (Sheves M. and Mazur Y. (1974) J. Am. Chem. Soc. 97:6249-6250; Paaren H. E. et al. (1980) J. Org. Chem. 45:3253-3258; Kabat M. et al. (1991) Tetrahed Lett 32:2343-2346; Wilson S. R. et al. (1991) Tetrahed Lett 32:2339-2342); the direct modification of vitamin D derivatives to 1-oxygenated 5,6-trans vitamin D as described in (Andrews D. R. et al. (1986) J. Org. Chem. 51:1635-1637); the Diels-Alders cycloadduct method of previtamin D₃ can be used to cyclorevert to 1-alpha,25(OH)₂D₂ through the intermediary of a previtamin form via thermal isomerization (Vanmaele L. et al. (1985) Tetrahedron 41:141-144); and, a final method entails the direct modification of 1-alpha,25(OH)₂D₂ or an analogue through use of suitable protecting groups such as transition metal derivatives or by other chemical transformations (Okarmura W. H. et al. (1992) J. Cell Biochem. 49:10-18). Additional methods for synthesizing vitamins D2 compounds are described in, for example, Japanese Patent Disclosures Nos. 62750173, 26858/76, 26859/76, and 71456/77; U.S. Pat. Nos. 3,639,596; 3,715,374; 3,847,955 and 3,739,001.

Examples of the compounds of this invention having a saturated side chain can be prepared according to the general process illustrated and described in U.S. Pat. No. 4,927,815. Examples of compounds of the invention having an unsaturated side chain can be prepared according to the general process illustrated and described in U.S. Pat. No. 4,847,012. Examples of compounds of the invention wherein R groups together represent a cycloalkyl group can be prepared according to the general process illustrated and described in U.S. Pat. No. 4,851,401.

Another synthetic strategy for the preparation of side-chain-modified analogues of 1-alpha,25-dihydroxyergocalciferol is disclosed in Kutner et al., The Journal of Organic Chemistry, 1988, 53:3450-3457. In addition, the preparation of 24-homo and 26-homo vitamin D analogues are disclosed in U.S. Pat. No. 4,717,721.

The enantioselective synthesis of chiral molecules is now state of the art. Through combinations of enantioselective synthesis and purification techniques, many chiral molecules can be synthesized as an enantiomerically enriched preparation. For example, methods have been reported for the enantioselective synthesis of A-ring diastereomers of 1-alpha,25(OH)₂D₃ as described in Muralidharan et al. (1993) J. Organic Chem. 58(7): 1895-1899 and Norman et al. (1993) J. Biol. Chem. 268(27): 20022-30. Other methods for the enantiomeric synthesis of various compounds known in the art include, inter alia, epoxides (see, e.g., Johnson, R. A.; Sharpless, K. B. In Catalytic Asymmetric Synthesis; Ojima, I., Ed.: VCH: New York, 1993; Chapter 4.1. Jacobsen, E. N. Ibid. Chapter 4.2), diols (e.g., by the method of Sharpless, J. Org. Chem. (1992) 57:2768), and alcohols (e.g., by reduction of ketones, E. J. Corey et al., J. Am. Chem. Soc. (1987) 109:5551). Other reactions useful for generating optically enriched products include hydrogenation of olefins (e.g., M. Kitamura et al, J. Org. Chem. (1988) 53:708); Diels-Alder reactions (e.g., K. Narasaka et al., J. Am. Chem. Soc. (1989) 111:5340); aldol reactions and alkylation of enolates (see, e.g., D. A. Evans et al., J. Am. Chem. Soc. (1981) 103:2127; D. A. Evans et al., J. Am. Chem. Soc. (1982) 104:1737); carbonyl additions (e.g., R. Noyori, Angew. Chem. Int. Ed. Eng. (1991) 30:49); and ring-opening of meso-epoxides (e.g., Martinez, L. E.; Leighton J. L., Carsten, D. H.; Jacobsen, E. N. J. Am. Chem. Soc. (1995) 117:5897-5898). The use of enzymes to produce optically enriched products is also well known in the art (e.g., M. P. Scheider, ed. “Enzymes as Catalysts in Organic Synthesis”, D. Reidel, Dordrecht (1986).

Chiral synthesis can result in products of high stereoisomer purity. However, in some cases, the stereoisomer purity of the product is not sufficiently high. The skilled artisan will appreciate that the separation methods described herein can be used to further enhance the stereoisomer purity of the vitamin D₃-epimer obtained by chiral synthesis.

Compounds of formula (XVIII):

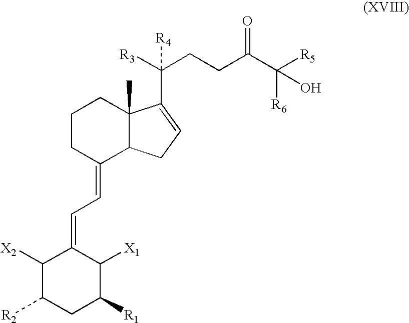

wherein:
X₁ and X₁ are each independently H₂ or ═CH₂, provided X₁ and X₁ are not both ═CH₂;
R₁ and R₂ are each independently, hydroxyl, OC(O)C₁-C₄ alkyl, OC(O)hydroxyalkyl, OC(O)fluororalkyl, provided that R₁ and R₂ are not both hydroxyl;
R₃ and R₄ are each independently hydrogen, C₁-C₄ alkyl, or R₃ and R₄ taken together with C20 form C₃-C₆ cycloalkyl; and
R₅ and R₆ are each independently C₁-C₄ alkyl, hydroxyalkyl, or haloalkyl, e.g., fluoroalkyl, e.g., fluoromethyl and trifluoromethyl;
   and pharmaceutically acceptable esters, salts, and prodrugs thereof, can be synthesized by methods described in this section, and the chemical literature. In particular, compounds of formula (XVIII) of the invention are prepared as shown in Scheme 1 below.

Accordingly, compounds of formula (XVIII) are prepared by coupling compounds of formula (XIX) with compounds of formula (XX) in tetrahydrofuran with n-butyllithium as a base to give compounds of formula (XXI). Subsequent removal of the protecting silyl groups (R₁=OSi(CH₃)₂t.Bu) affords the 1,3 dihydroxy vitamin D₃ compound of formula (XVIII) (R₁═OH, R₂═OH). Acylation at the 1 and/or 3 positions is achieved using methods well-known in the art. For example, preparation of the 1,3 diacetoxy compounds of formula IV (R₁═R₂═OAc) requires additional acetylation with acetic anhydride and pyridine, as shown in Scheme 2 and described below.

Referring to Scheme 1, compounds of formula (XX) are known compounds, and are prepared starting from the known epoxy-ketone of formula (XXII). The compound of formula (XXII) is converted to the epoxy-olefin of formula (XXIII) by a Wittig reaction. Reduction with LiAlH₄ to the compound (XXIV) and protection of the hydroxy group resulted in compound (XXV). Then, the ene reaction of formula (XXV) with the known hydroxy-conjugated ketone (XXVI) (R₅=═CH₃) in tetrahydrofuran, in the presence of Lewis acid (CH₃)₂ Al Cl, provides the compound (XXVII) featuring the C,D-rings and full side chain of the target vitamin D analogs. Finally, removal of the silyl group and oxidation provides the key intermediate, Ketone of formula (XX).

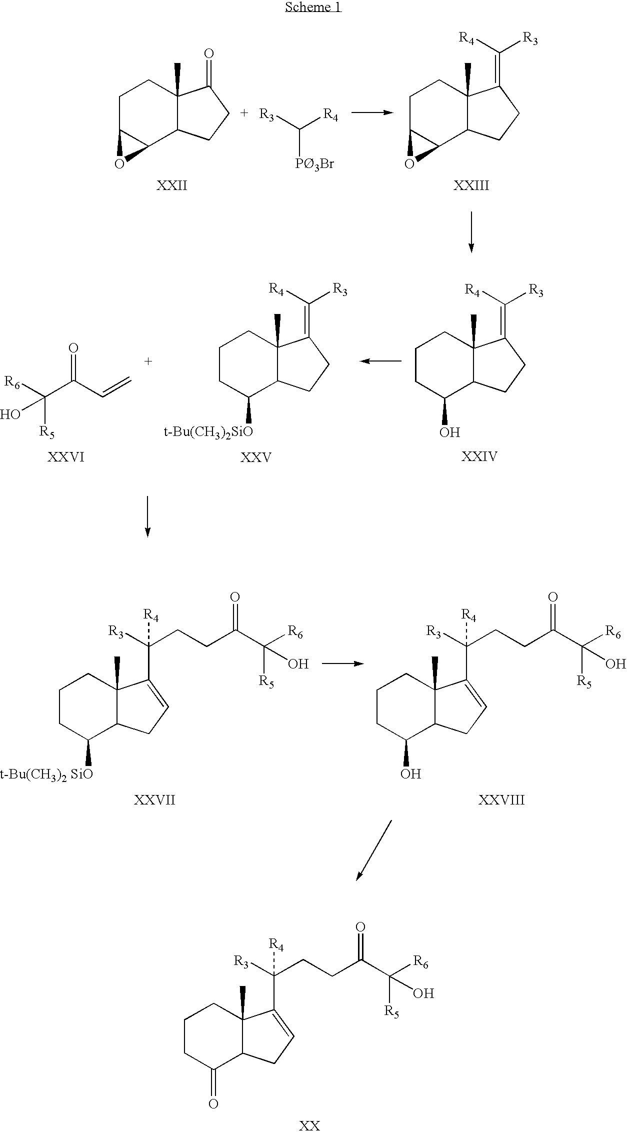

Scheme 2 shows the coupling of compound (XX) with a silylated phosphine oxide under Witting coupling conditions. Removal of the silyl protecting group provides diols of formula (XVIII), where R₁ and R² are both hydroxyl.

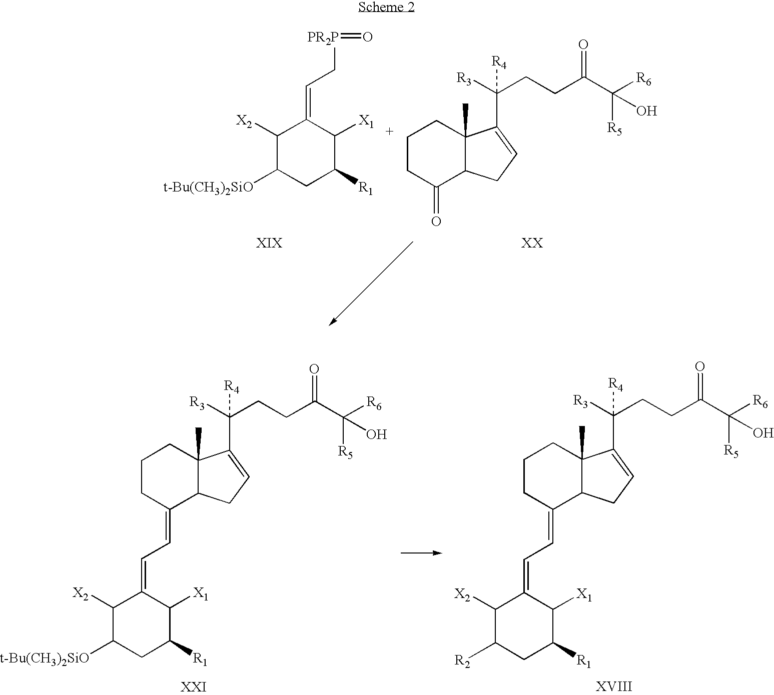

wherein X₁, X₂, R₃, R₄, R₅ and R₆ are as defined above.

Scheme 3 demonstrates the acetylation of the vitamin D₃ derivatives of formula (P) to the acetates of formula (Q).

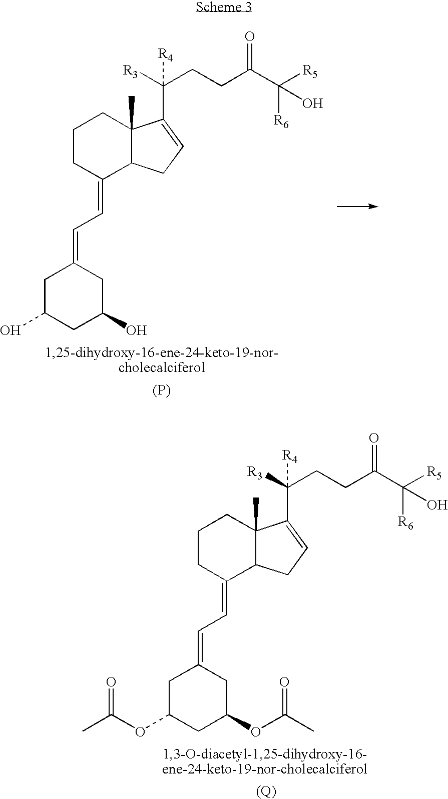

Vitamin D₃ compounds of the formula:

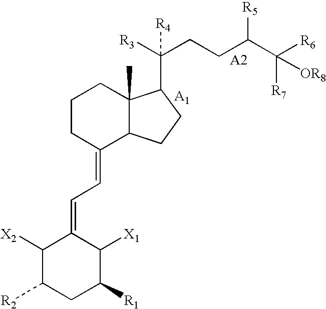

wherein:
A₁ is single or double bond;
A₂ is a single, double or triple bond;
X₁ and X₂ are each independently H or ═CH₂,
R₁ and R₂ are each independently OC(O)C₁-C₄ alkyl, OC(O)hydroxyalkyl, or OC(O)haloalkyl;
R₃, R₄ and R₅ are each independently hydrogen, C₁-C₄ alkyl, hydroxyalkyl, or haloalkyl, or R₃ and R₄ taken together with C20 form C₃-C₆ cycloalkyl;
R₆ and R₇ are each independently haloalkyl; and
R₈ is H or C(O)C₁-C₄ alkyl, C(O)hydroxyalkyl, or OC(O)haloalkyl; and pharmaceutically acceptable esters, salts, and prodrugs thereof.
   may be prepared analogously to the synthesis of 1,3-Di-O-acetyl-1,25-dihydroxy-16,23Z-diene-26,27-hexafluoro-19-nor-cholecalciferol (1), which is carried out under standard acetylation conditions of the diol to the corresponding diacetate:

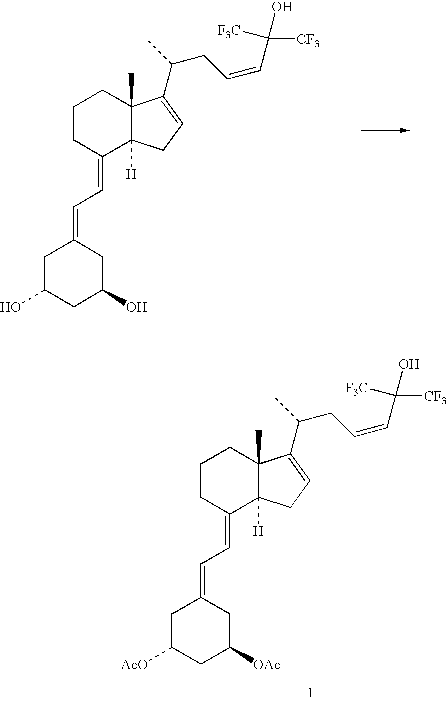

The present invention will now be described with reference to the following non-limiting examples, with reference to the figures, in which:

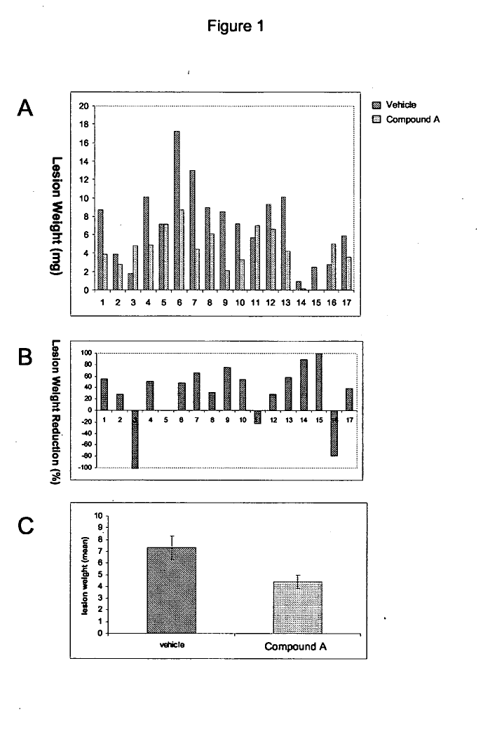

FIG. 1 shows the effect of treatment with a vitamin D compound (Compound A) on lesion weight in an in vivo model of endometriosis. Panel A—pairs of treated and untreated subjects receiving the same donor cells. Panel B—change in lesion weight for specific pairs. Panel C—Average lesion weight in treated and untreated subjects.

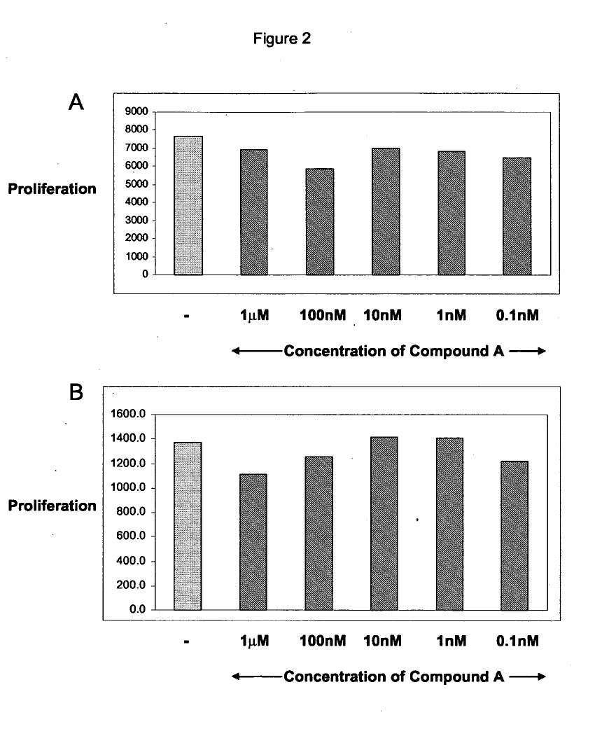

FIG. 2 shows the effect of treatment with a vitamin D compound (Compound A) on the proliferation of endometrial stromal cells. Panel A—Eutopic cells, Panel B—Ectopic cells.

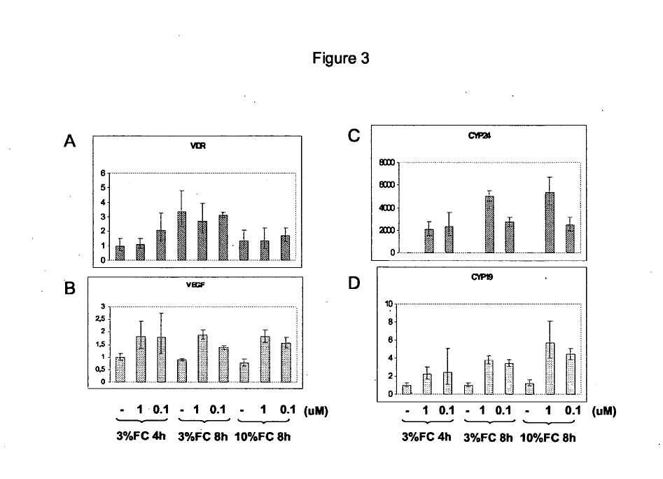

FIG. 3 shows the effect of treatment with a vitamin D compound (Compound A) on gene expression in cultured cells.

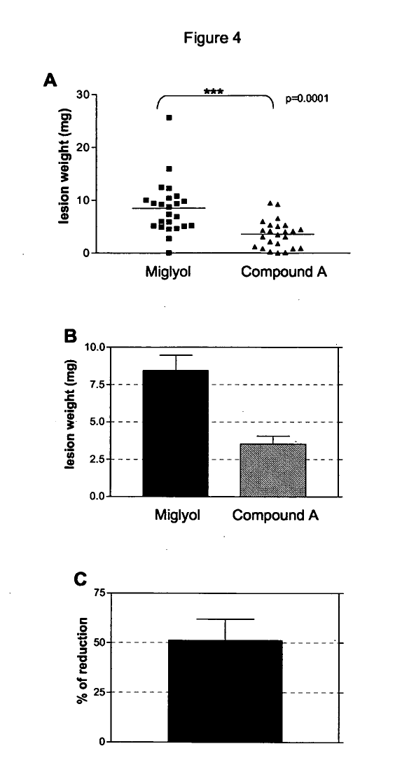

FIG. 4 shows the effect of treatment with a vitamin D compound (Compound A) on lesion weight in an in vivo model of endometriosis. Panel A—complete data set. Panel B—average lesion weight for treatment groups. Panel C—Relative reduction in lesion weight as a result of treatment.

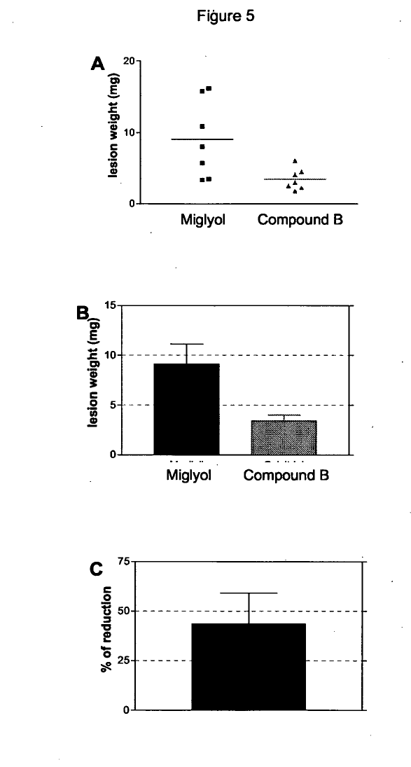

FIG. 5 shows the effect of treatment with a vitamin D compound (Compound B) on lesion weight in an in vivo model of endometriosis. Panel A—complete data set. Panel B—average lesion weight for treatment groups. Panel C—Relative reduction in lesion weight as a result of treatment.

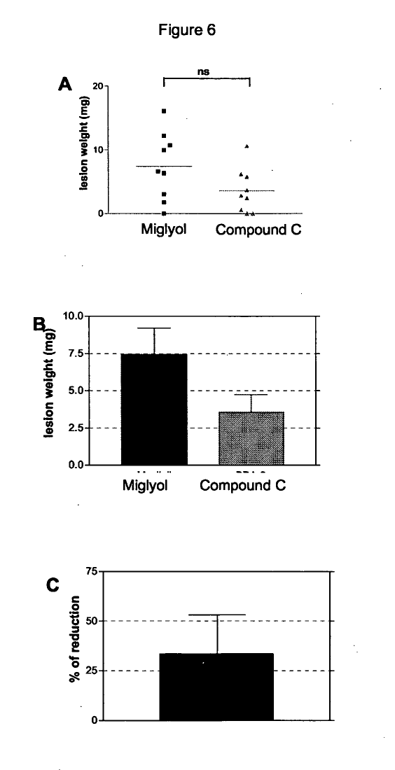

FIG. 6 shows the effect of treatment with a vitamin D compound (Compound C) on lesion weight in an in vivo model of endometriosis. Panel A—complete data set. Panel B—average lesion weight for treatment groups. Panel C—Relative reduction in lesion weight as a result of treatment.

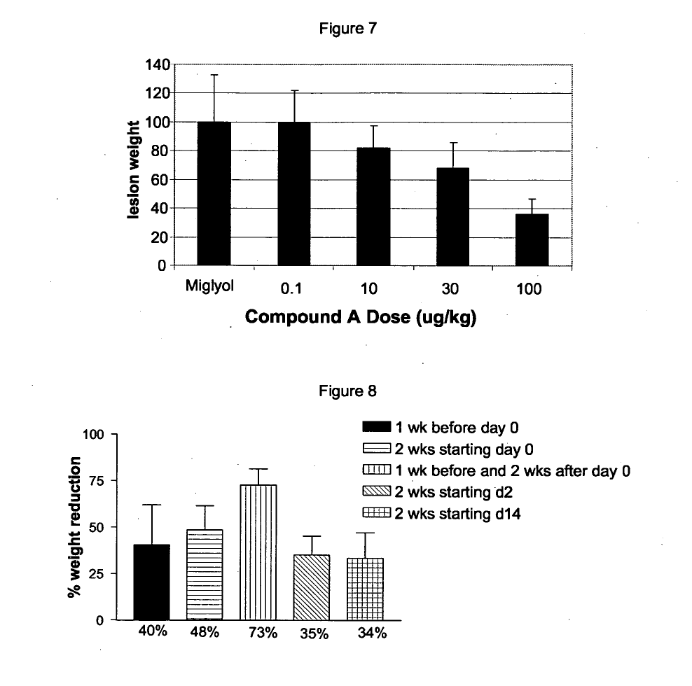

FIG. 7 illustrates the reduction in lesion weight as a function of different dosages of the vitamin D compound Compound A.

FIG. 8 illustrates the reduction in lesion weight resulting from a range of different treatment regimes using the vitamin D compound Compound A.

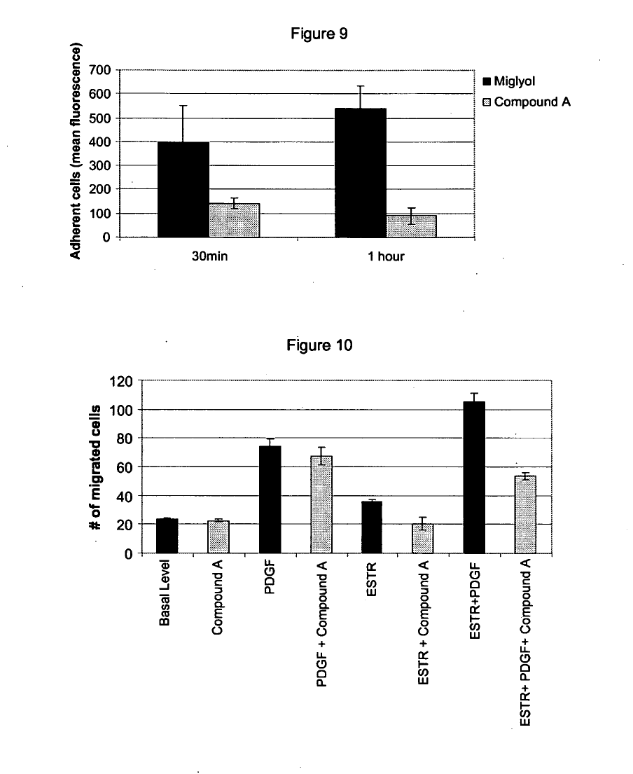

FIG. 9 shows the effect of treatment with Compound A on cell adhesion.

FIG. 10 shows the effect of treatment with Compound A on cell migration.

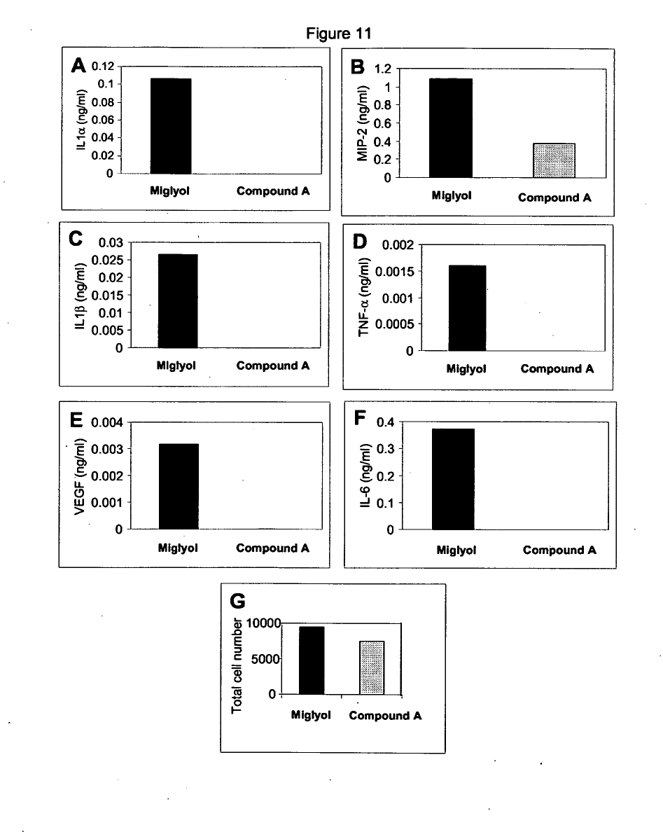

FIG. 11 shows the effect of treatment with Compound A on a range of inflammatory markers.

### SYNTHETIC EXAMPLES

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 μm mesh silica gel. Preparative HPLC was performed on a 5×50 cm column and 15-30 μm mesh silica gel at a flow rate of 100 ml/min.

### Synthetic Example 1

### Synthesis of 1,3-Di-O-acetyl-1,25-dihydroxy-16,23Z-diene-26,27-hexafluoro-19-nor-cholecalciferol (1)

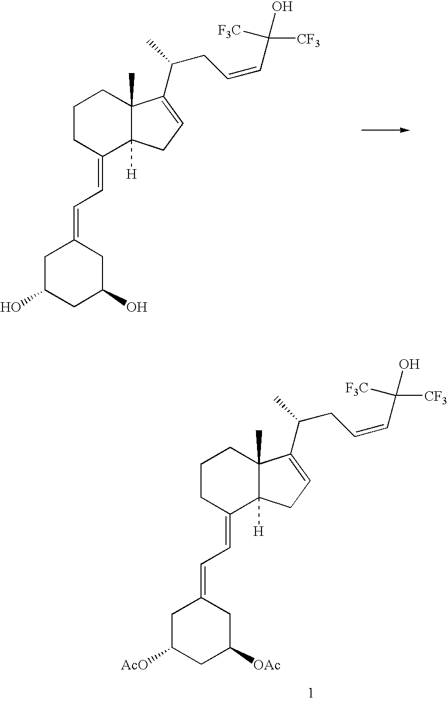

The starting material 1,25-dihydroxy-16,23Z-diene-26,27-hexafluoro-19-nor-cholecalciferol can be prepared as described in U.S. Pat. No. 5,428,029 to Doran et al. 3 mg of 1,25-dihydroxy-16,23Z-diene-26,27-hexafluoro-19-nor-cholecalciferol was dissolved in 0.8 ml of pyridine, cooled to ice-bath temperature and 0.2 ml of acetic anhydride was added and maintained at that temperature for 16 h. Then the reaction mixture was diluted with 1 ml of water, stirred for 10 min in the ice bath and distributed between 5 ml of water and 20 ml of ethyl acetate. The organic layer was washed with 3×5 ml of water, once with 5 ml of saturated sodium hydrogen carbonate, once with 3 ml of brine then dried (sodium sulfate) and evaporated. The oily residue was taken up in 1:6 ethyl acetate-hexane and flash-chromatographed using a stepwise gradient of 1:6, 1:4 and 1:2 ethyl acetate-hexane. The column chromatography was monitored by TLC (1:4 ethyl acetate-hexane, spot visualization with phosphomolybdic acid spray), the appropriate fractions were pooled, evaporated, the residue taken up in methyl formate, filtered, then evaporated again to give 23.8 mg of the title compound (1) as a colorless syrup; 400 MHz ¹H NMR δ 0.66 (3H, s), 0.90 (1H, m), 1.06 (3H, d, J=7.2 Hz), 1.51 (1H, m), 1.72-1.82 (3H, m), 1.9-2.1 (3H, m), 1.99 (3H, s) 2.04 (3H, s), 2.2-2.3 (3 m), 2.44-2.64 (6H, m), 2.78 (1H, m), 3.01 (1H, s), 5.10 (2H, m). 5.38 (1H, m), 5.43 (1H, d, J=12 Hz), 5.85 (1H, d, J=11.5 Hz), 5.97 (1H, dt, J=12 and 7.3 Hz), 6.25 (1H, d, J=11.5 Hz).

### Synthetic Example 2

### Synthesis of 1,3-Di-O-acetyl-1,25-Dihydroxy-16-ene-23-yne-26,27-hexafluoro-19-nor-cholecalciferol (2) and 1,3,25-Tri-O-acetyl-1,25-Dihydroxy-16-ene-23-yne-26,27-hexafluoro-19-nor-cholecalciferol (3)

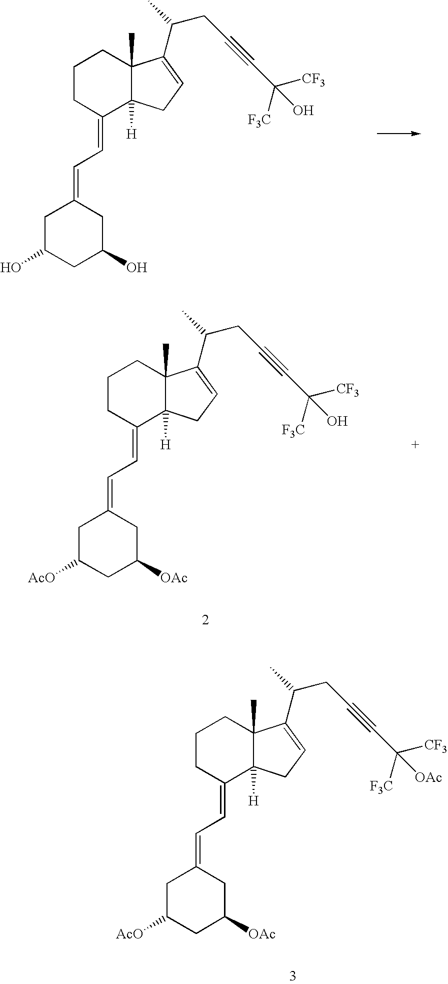

The starting material 1,25-dihydroxy-16-ene-23-yne-26,27-hexafluoro-19-nor-cholecalciferol can be prepared as described in U.S. Pat. Nos. 5,451,574 and 5,612,328 to Baggiolini et al. 314 mg (0.619 mmole) of 1,25-dihydroxy-16-ene-23-yne-26,27-hexafluoro-19-nor-cholecalciferol was dissolved in 1.5 ml of pyridine, cooled to ice-bath temperature, and 0.4 ml of acetic anhydride was added. The reaction mixture was kept at room temperature for 7 hours and then for 23 hours in a refrigerator. It was then diluted with 10 ml water and extracted with 30 ml of ethyl acetate. The organic extract was washed with water and brine, dried over sodium sulfate and evaporated. The residue was FLASH chromatographed on a 10×140 mm column with 1:6 and 1:4 ethyl acetate-hexane as the mobile phase to give 126 mg of 1,3-Di-O-acetyl-1,25-Dihydroxy-16-ene-23-yne-26,27-hexafluoro-19-nor-cholecalciferol (2); and 248 mg of 1,3,25-Tri-O-acetyl-1,25-Dihydroxy-16-ene-23-yne-26,27-hexafluoro-19-nor-cholecalciferol (3).

### Synthetic Example 3

### Synthesis of 1,3-Di-O-acetyl-1,25-dihydroxy-16-ene-23-yne-cholecalciferol (4)

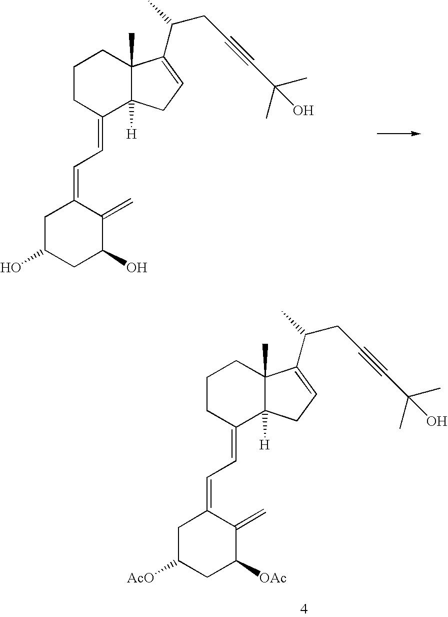

A 10-mL round-bottom flask was charged with 40 mg of 1,25-dihydroxy-16-ene-23-yne-cholecalciferol. This material was dissolved in 1 mL of pyridine. This solution was cooled in an ice bath then 0.3 mL of acetic anhydride was added. The solution was stirred for 30 min, then refrigerated overnight, diluted with water and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-5, 1:6 for fractions 6-13 and 1:4 ethyl acetate-hexane for fractions 14-20 (18 mL fractions). Fractions 14-19 contained the main band with Rf0.15 (TLC 1:4). Those fractions were pooled and evaporated to a colorless oil, 0.044 g. The material was taken up in methyl formate, filtered and evaporated to give a colorless, sticky foam, 0.0414 g of the title compound (4).

### Synthetic Example 4

### Synthesis of 1,3-Di-O-acetyl-1,25-dihydroxy-16,23E-diene-cholecalciferol (5)

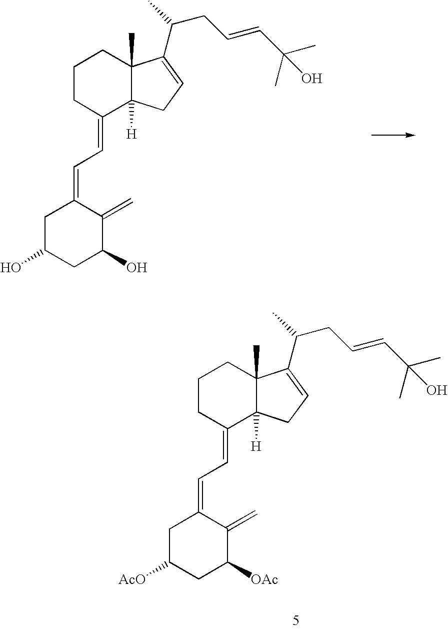

0.0468 g of 1,25-Dihydroxy-16,23E-diene-cholecalciferol was dissolved in 1.5 mL of pyridine. This solution was cooled in an ice bath then refrigerated overnight, diluted with 10 mL of water while still immersed in the ice bath, stirred for 10 min and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-3 (20 mL fractions), 1:6 for fractions 6-8 and 1:4 ethyl acetate-hexane for fractions 9-17 (18 mL each). Fractions 11-14 contained the main band with Rf 0.09 (TLC 1:4). Those fractions were pooled and evaporated to a colorless oil, 0.0153 g. This material was taken up in methyl formate, filtered and evaporated, to give 0.014 g of the title compound (5).

### Synthetic Example 6

### Synthesis of 1,3-Di-O-acetyl-1,25-dihydroxy-16-one-cholecalciferol (6)

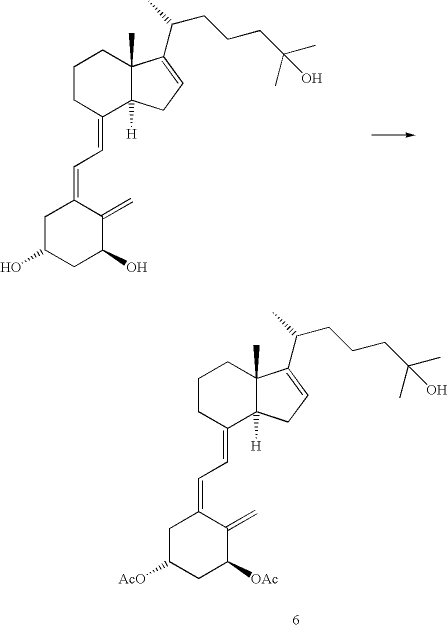

0.0774 g of 1,25-Dihydroxy-16-ene-cholecalciferol was dissolved in 1.5 mL of pyridine. This solution was cooled in an ice bath then 0.3 mL of acetic anhydride was added. The solution was stirred, refrigerated overnight then diluted with 1 mL of water, stirred for 1 h in the ice bath and diluted with 30 mL of ethyl acetate and 15 mL of water. The organic layer was washed with 4×15 mL of water, once with 5 mL of brine then dried (sodium sulfate) and evaporated. The light brown, oily residue was taken up in 1:9 ethyl acetate-hexane then flash chromatographed on a 10×130 mm column using 1:9 ethyl acetate-hexane as mobile phase for fraction 1 (20 mL fractions), 1:6 for fractions 2-7 and 1:4 ethyl acetate-hexane for fractions 8-13. Fractions 9-11 contained the main band with Rf 0.09 (TLC 1:4 ethyl acetate-hexane). Those fractions were pooled and evaporated to a colorless oil, 0.0354 g. This material was taken up in methyl formate, filtered and the solution evaporated, 0.027 g colorless film, the title compound (6).

### Synthetic Example 7

### Synthesis of 1,3,25-Tri-O-acetyl-1,25-dihydroxy-16-ene-23-yne-26,27-hexafluoro-cholecalciferol (7) and 1,3-Di-O-acetyl-1,25-dihydroxy-16-ene-23-yne-26,27-hexafluoro-cholecalciferol (8)

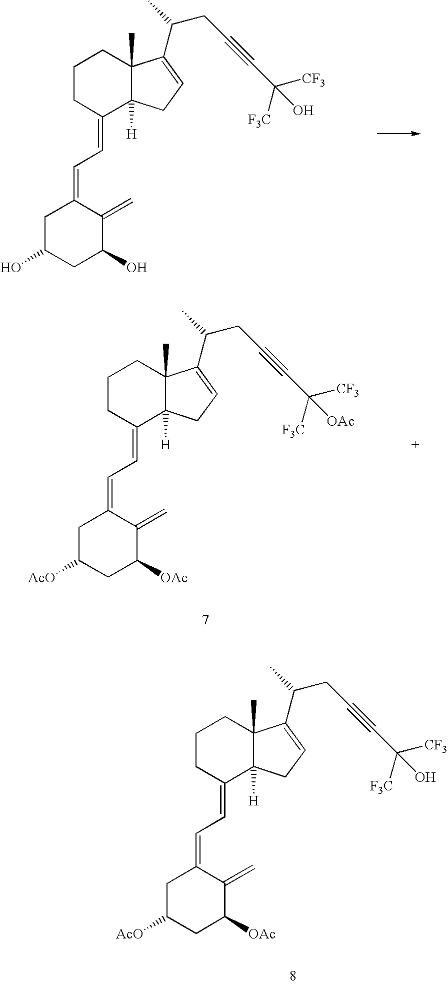

0.0291 g of 1,25-dihydroxy-16-ene-23-yne-26,27-hexafluoro-cholecalciferol was dissolved in 1.5 mL of pyridine. This solution was cooled in an ice bath then 0.25 mL of acetic anhydride was added. The solution was stirred for 20 min and kept in a freezer overnight. The cold solution was diluted with 15 mL of water, stirred for 10 min, and diluted with 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×110 mm column using 1:6 ethyl acetate-hexane as mobile phase. Fractions 2-3 gave 72.3461-72.3285=0.0176 g. Evaporation of fractions 6-7 gave 0.0055 g. The residue of fractions 2-3 was taken up in methyl formate, filtered and evaporated to give 0.0107 g of the title triacetate (7). The residue of fractions 6-7 was taken up in methyl formate, filtered and evaporated to give 0.0049 g of diacetate (8).

### Synthetic Example 8

### Synthesis of 1,3-Di-O-acetyl-1,25-dihydroxy-16,23E-diene-25R,26-trifluoro-cholecalciferol (9)

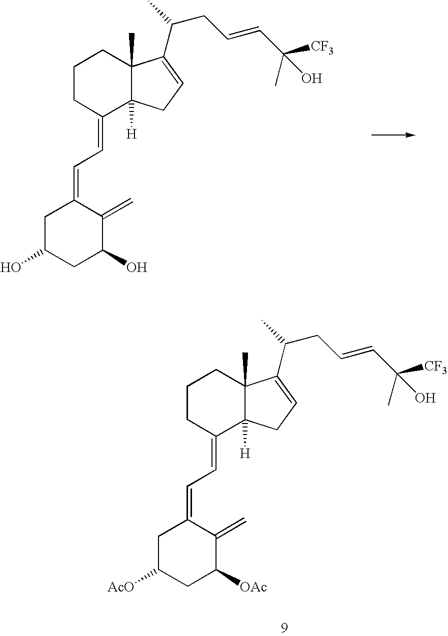

1.5 mL of 1,25-dihydroxy-16,23E-diene-25R,26-trifluoro-cholecalciferol was dissolved in 1.5 mL of pyridine, cooled to ice-bath temperature and 0.4 mL of acetic anhydride was added. The mixture was then refrigerated. After two days the mixture was diluted with 1 mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×130 mm column using 1:6 ethyl acetate-hexane as mobile phase. Fractions 4-6 (TLC, 1:4) contained the main band (see TLC) These fractions were evaporated and gave 0.0726 g. This residue was taken up in methyl formate, filtered and evaporated, to give 0.0649 g of colorless foam, the title compound (9).

### Synthetic Example 8

### Synthesis of 1,3-Di-O-acetyl-1,25-dihydroxy-16-ene-19-nor-cholecalciferol (10)

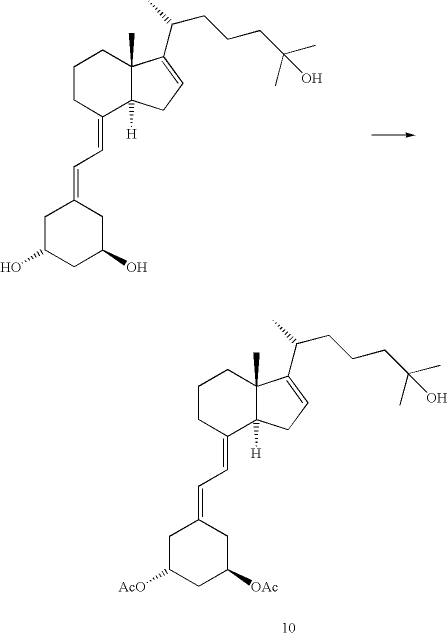

0.0535 g of 1,25-Dihydroxy-16-ene-19-nor-cholecalciferol was dissolved in 1.5 mL of pyridine, cooled to ice-bath temperature and 0.3 mL of acetic anhydride was added and the mixture was refrigerated overnight. The solution was diluted with 1 mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The nearly colorless, oily residue was taken up in 1:6 ethyl acetate-hexane as mobile phase for fractions 1-6 then 1:4 ethyl acetate-hexane was used. Fractions 9-19 (TLC, 1:4 ethyl acetate-hexane, Rf 0.09, see below) were pooled, evaporated, to give 0.0306 g, which was taken up in methyl formate, filtered, then evaporated. It gave 0.0376 of the title compound (10).

### Synthetic Example 9

### Synthesis of 1,3-Di-O-Acetyl-1,25-dihydroxy-16-ene-23-yne-19-nor-cholecalciferol (11)

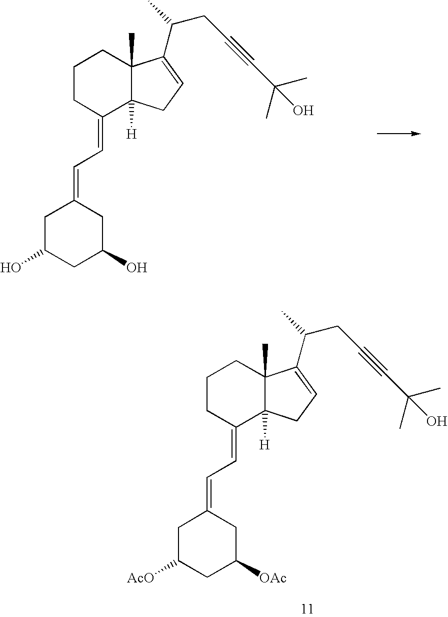

50 mg of 1,25-dihydroxy-16-ene-23-yne-19-nor-cholecalciferol was dissolved in 0.8 mL of pyridine, cooled to ice-bath temperature and 0.2 mL of acetic anhydride was added. The mixture was refrigerated for 3 days then diluted with 1 mL of water, stirred for 10 min in the ice bath and distributed between 5 mL of water and 20 mL of ethyl acetate. The organic layer was washed with 4×5 mL of water, once with 3 mL of brine then dried (sodium sulfate) and evaporated. The nearly colorless, oily residue was taken up in 1:6 ethyl acetate-hexane then flash chromatographed on a 15×120 mm column using 1:6 ethyl acetate-hexane as mobile phase for fractions 1-6, 1:4 for fractions 9-12, 1:3 for fractions 13-15 and 1:2 ethyl acetate-hexane for the remaining fractions. Fractions 11-16 (TLC, 1:4 ethyl acetate-hexane, Rf 0.09, see below) were pooled, evaporated 76.1487-76.1260=0.0227 g, taken up in methyl formate, filtered, then evaporated. It gave 0.0186 g of the title compound (11).

### Synthetic Example 10

### Synthesis of 1,3-Di-O-acetyl-1,25-dihydroxy-16-ene-23-yne-26,27-bishomo-19-nor-cholecalciferol (12)

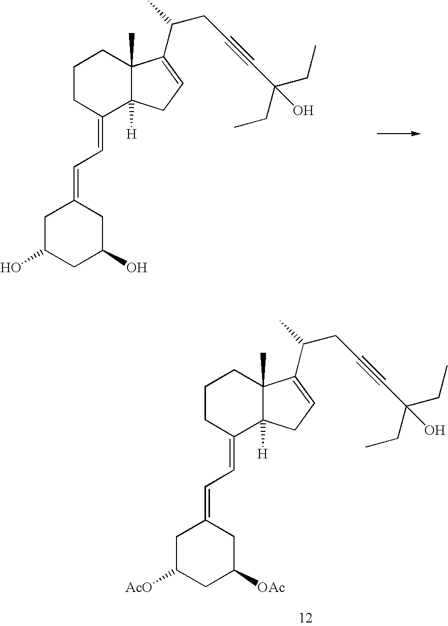

0.0726 g of 1,25-dihydroxy-16-ene-23-yne-26,27-bishomo-19-nor-cholecalciferol was dissolved in 0.8 mL of pyridine, cooled to ice-bath temperature and 0.2 mL of acetic anhydride was added. The solution was stirred in the ice-bath then refrigerated overnight. The solution was then diluted with 1 mL of water, stirred for 10 min in the ice bath and distributed between 10 mL of water and 25 mL of ethyl acetate. The organic layer was washed with 3×10 mL of water, once with 5 mL of saturated sodium hydrogen carbonate, once with 3 mL of brine then dried and evaporated, 33.5512-33.4654=0.0858 g of a tan oily residue that was flash-chromatographed on a 15×120 mm column using 1:6 as mobile phase. Fractions 7-11 (20 mL each) were pooled (TLC 1:4 ethyl acetate-hexane, Rf 0.14) and evaporated, 67.2834-67.2654=0.018 g. This residue was taken up in methyl formate, filtered and evaporated. It gave 0.0211 g of the title compound (12).

### Synthetic Example 11

### Synthesis of 1,3-Di-O-acetyl-1,25-dihydroxy-20-cyclopropyl-23-yne-19-nor-cholecalciferol (13)

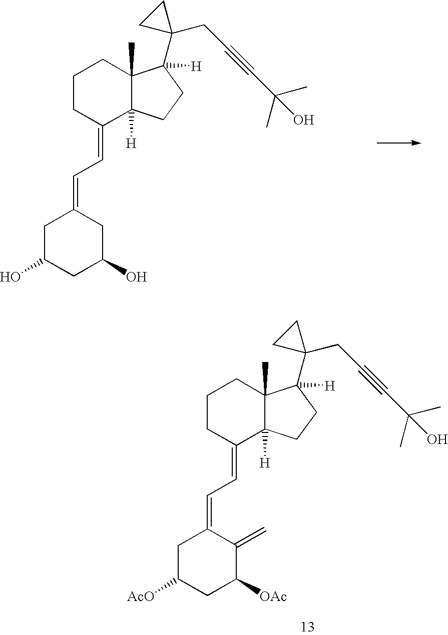

0.282 g of 1,25-Dihydroxy-20-cyclopropyl-23-yne-19-nor-cholecalciferol was dissolved in 0.8 mL of pyridine, cooled to ice-bath temperature and 0.2 mL of acetic anhydride was added and the mixture was refrigerated overnight, then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×110 mm column using 1:6 ethyl acetate-hexane as mobile phase for fractions 1-4, 1:4 for fractions 5-12, 1:3 for fractions 13-15 ethyl acetate-hexane for the remaining fractions. Fractions 7-12 (TLC, 1:4 ethyl acetate-hexane, Rf 0.13) were pooled, evaporated, the residue taken up in methyl formate, filtered, then evaporated to give 0.023 g of the title compound (13).

### Synthetic Example 12

### Synthesis of 1,3,25-Tri-O-acetyl-1,25-dihydroxy-20-cyclopropyl-23-yne-26,27-hexafluoro-19-nor-cholecalciferol (14) and 1,3-Di-O-acetyl-1,25-dihydroxy-20-cyclopropyl-23-yne-26,27-hexafluoro-19-nor-cholecalciferol (15)

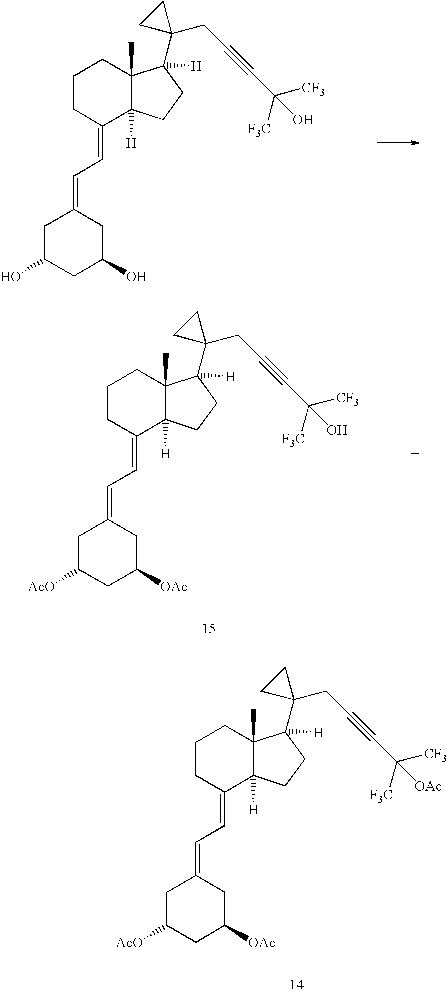

0.1503 g of 1,25-dihydroxy-20-cyclopropyl-23-yne-26,27-hexafluoro-19-nor-cholecalciferol was dissolved in 0.8 mL of pyridine, cooled to ice-bath temperature and 0.2 mL of acetic anhydride was added. The mixture was refrigerated overnight then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×150 mm column using 1:6 ethyl acetate-hexane as mobile phase for fractions 1-5, 1:4 for the remaining fractions. Fractions 3-4 and 6-7 were pooled, evaporated, then taken up in methyl formate, filtered, and evaporated to give 0.0476 g of the title triacetate (14) and 0.04670 g of the title diacetate (15).

### Synthetic Example 13

### Synthesis of 1,3-Di-O-acetyl-1,25-dihydroxy-20-cyclopropyl-23-yne-cholecalciferol (16)

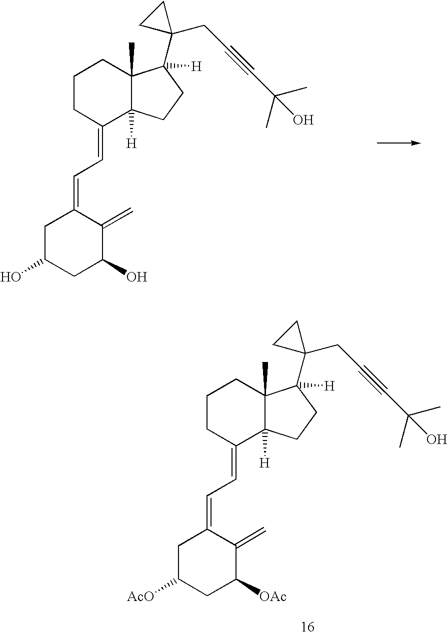

0.0369 g of 1,25-dihydroxy-20-cyclopropyl-23-yne-cholecalciferol was dissolved in 0.8 mL of pyridine, cooled to ice-bath temperature and 0.2 mL of acetic anhydride was added and the mixture was refrigerated overnight, then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash-chromatographed on a 13×110 mm column using 1:6 ethyl acetate-hexane as mobile phase for fractions 1-7, 1:4 ethyl acetate-hexane for the remaining fractions. Fractions 9-11 (TLC, 1:4 ethyl acetate-hexane) were pooled, evaporated, taken up in methyl formate, filtered, then evaporated, to give 0.0099 g of the title compound (16).

### Synthetic Example 14

### Synthesis of 1,3-Di-O-acetyl-1,25-dihydroxy-20-cyclopropyl-23E-ene-26,27-hexafluoro-19-nor-cholecalciferol (17)

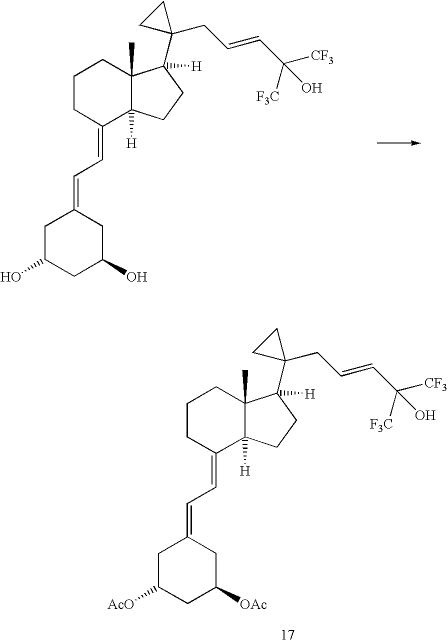

0.0328 g of 1,25-dihydroxy-20-cyclopropyl-23E-ene-26,27-hexafluoro-19-nor-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 5 mL of water and 20 mL of ethyl acetate. (Extraction of the aqueous layer gave no phosphomolybdic acid-detectable material). The organic layer was washed with 3×5 mL of water, once with 5 mL of saturated sodium hydrogen carbonate, once with 3 mL of brine then dried (sodium sulfate) and evaporated, the residue shows Rf 0.25 as the only spot. The oily residue was taken up in 1:6 ethyl acetate-hexane then flash-chromato-graphed on a 13.5×110 mm column using 1:6 ethyl acetate-hexane as mobile phase for fractions 1-10. Fractions 4-9 were pooled and evaporated, the residue taken up in methyl formate, filtered, then evaporated to give 0.0316 g of the title compound (17).

### Synthetic Example 15

### Synthesis of 1,3-Di-O-acetyl-1,25-dihydroxy-20-cyclopropyl-23Z-ene-26,27-hexafluoro-19-nor-cholecalciferol (18)

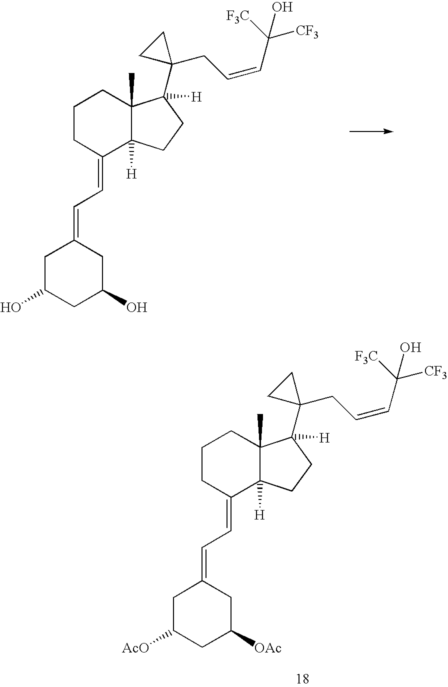

0.0429 g of 1,25-dihydroxy-20-cyclopropyl-23Z-ene-26,27-hexafluoro-19-nor-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 7 mL of water and 25 mL of ethyl acetate. The organic layer was washed with 3×5 mL of water, once with 5 mL of saturated sodium hydrogen carbonate, once with 3 mL of brine then dried (sodium sulfate, TLC (1:4 ethyl acetate-hexane shows mostly one spot) and evaporated, flash-chromatographed on a 15×120 mm column using 1:6 as mobile phase. Fractions 3-6 (20 mL each) were pooled and evaporated. The residue was taken up in methyl formate, filtered and evaporated, to give 0.0411 g of the title compound (18).

### Synthetic Example 16

### Synthesis of 1,3-Di-O-acetyl-1,25-dihydroxy-20-cyclopropyl-cholecalciferol (19)

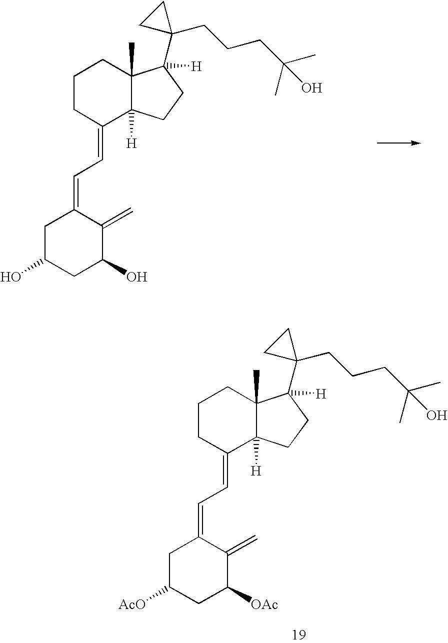

0.0797 g of 1,25-dihydroxy-20-cyclopropyl-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 10 mL of water and 25 mL of ethyl acetate. The organic layer was washed with 3×10 mL of water, once with 5 mL of saturated sodium hydrogen carbonate, once with 3 mL of brine then dried and evaporated, to give 0.1061 g of a tan oily residue that was flash-chromatographed on a 15×120 mm column using 1:6 as mobile phase. Fractions 9-16 (20 mL each) were pooled (TLC 1:4 ethyl acetate-hexane, Rf 0.13) and evaporated. This residue was taken up in methyl formate, filtered and evaporated to give 0.0581 g of the title compound (19).

### Synthetic Example 17

### Synthesis of 1,3-Di-O-acetyl-1-alpha,25-dihydroxy-16-ene-20-cyclopropyl-19-nor-cholecalciferol (20)

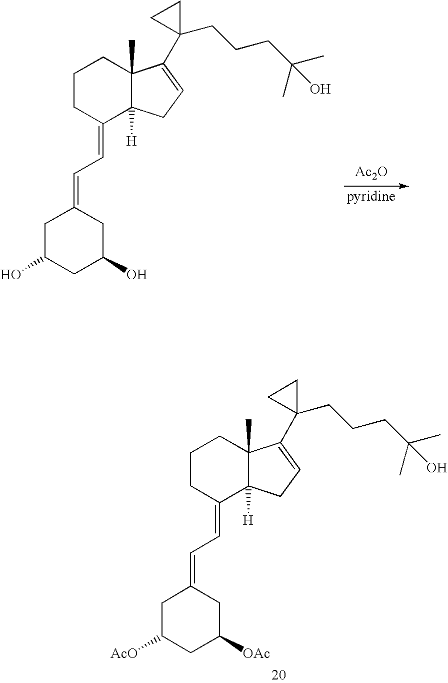

To the solution of 1-alpha,25-Dihydroxy-16-ene-20-cyclopropyl-19-nor-cholecalciferol (94 mg, 0.23 mmol) in pyridine (3 mL) at 0° C., acetic anhydride (0.5 mL, 5.3 mmol) was added. The mixture was stirred for 1 h, refrigerated for 15 h. and then was stirred for additional 8 h. Water (10 mL) was added and after stirring for 15 min. the reaction mixture was extracted with AcOEt: Hexane 1:1 (25 mL), washed with water (4×25 mL) and brine (20 mL), dried over Na₂SO₄. The residue (120 mg) after evaporation of the solvent was purified by FC (15 g, 30% AcOEt in hexane) to give the titled compound (20) (91 mg, 0.18 mmol, 80%). [α]³⁰_{D}=+ 14.4 c 0.34, EtOH; UV λmax (EtOH): 242 nm (ε34349 . . . ), 250en40458), 260 nm (ε 27545); ¹H NMR (CDCl₃): 6.25 (1H, d, J=11.1 Hz), 5.83 (1H, d, J=11.3 Hz), 5.35 (1H, m), 5.09 (2H, m), 2.82-1.98 (7H, m), 2.03 (3H, s), 1.98 (3H, s), 2.00-1.12 (15H, m), 1.18 (6H, s), 0.77 (3H, s), 0.80-0.36 (4H, m); ¹³C NMR (CDCl₃): 170.73(0), 170.65 (0), 157.27 (0), 142.55 (0), 130.01 (0), 125.06 (1), 123.84 (1), 115.71 (1), 71.32 (0), 70.24 (1), 69.99 (1), 59.68 (1), 50.40 (0), 44.08 (2), 41.40(2), 38.37 (2), 35.96 (2), 35.80 (2), 32.93 (2), 29.48 (3), 29.31 (2), 28.71 (2), 23.71 (2), 22.50 (2), 21.56 (3), 21.51 (0), 21.44(3), 18.01 (3), 12.93 (2), 10.53 (2); MS HRES Calculated for C31H₄₆O₅ M+Na 521.3237, Observed M+Na 521.3233

### Synthetic Example 18

### Synthesis of 1,3-Di-O-acetyl-1-alpha,25-hydroxy-16-ene-20-cyclopropyl-cholecalciferol (21)

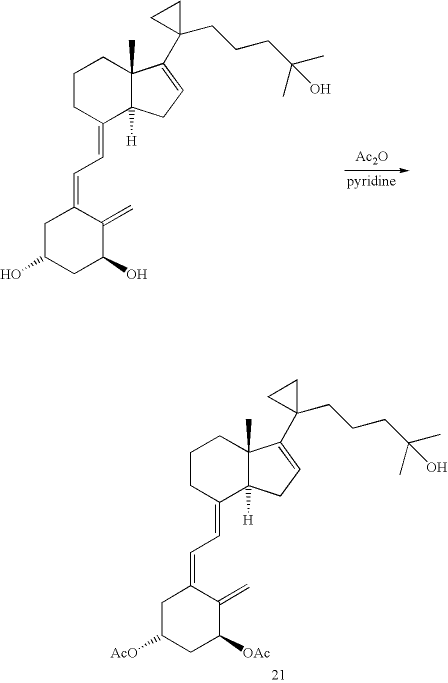

To the solution of 1-alpha,25-Dihydroxy-16-ene-20-cyclopropyl-cholecalciferol (100 mg, 0.23 mmol) in pyridine (3 mL) at 0° C., acetic anhydride (0.5 mL, 5.3 mmol) was added. The mixture was stirred for 2 h and then refrigerated for additional 15 h. Water (10 mL) was added and after stirring for 15 min. the reaction mixture was extracted with AcOEt:Hexane 1:1 (25 mL), washed with water (4×25 mL), brine (20 mL) and dried over Na₂SO₄. The residue (150 mg) after evaporation of the solvent was purified by FC (15 g, 30% AcOEt in hexane) to give the titled compound (21) (92 mg, 0.18 mmol, 78%). [α]³⁰_{D}=−14.9 c 0.37, EtOH; UV λmax (EtOH): 208 nm (ε 15949), 265 nm (ε 15745); ¹H NMR (CDCl₃): 6.34 (1H, d, J=11.3 Hz), 5.99 (1H, d, J=11.3 Hz), 5.47 (1H, m), 5.33 (1H, m), 5.31 (1H, s), 5.18 (1H, m), 5.04 (1H, s), 2.78 (1H, m), 2.64 (1H, m), 2.40-1.10 (18H, m), 2.05 (3H, s), 2.01 (3H, s), 1.18 (6H, s), 0.76 (3H, s), 0.66-0.24 (4H, m); ¹³C NMR (CDCl₃): 170.76(0), 170.22 (0), 157.18 (0), 143.02 (0), 142.40 (0), 131.94 (0), 125.31 (1), 125.10 (1), 117.40 (1), 115.22 (2), 72.97 (1), 71.32 (0), 69.65 (1), 59.71 (1), 50.57 (0), 44.07 (2), 41.73 (2), 38.36 (2), 37.10 (2), 35.80 (2), 29.45 (3), 29.35 (2), 29.25 (3), 28.92 (2), 23.80 (2), 22.48 (2), 21.55 (3), 21.50 (3), 21.35 (0), 17.90 (3), 12.92 (2), 10.54 (2); MS HRES Calculated for C₃₂H₄₆O₅ M+Na 533.3237, Observed M+Na 533.3236

### Synthetic Example 19

### Synthesis of 1,3-Di-O-acetyl-1,25-dihydroxy-23-yne-cholecalciferol (22)

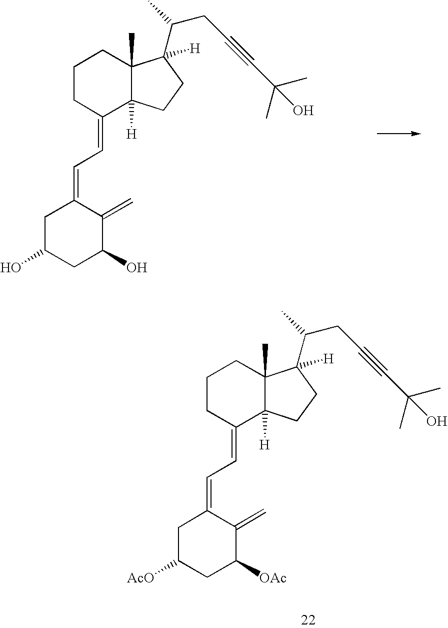

0.2007 g of (0.486 mmol) was dissolved in 2 mL of pyridine. This solution was cooled in an ice bath and 0.6 mL of acetic anhydride was added. The solution was kept in an ice bath for 45 h then diluted with 10 mL of water, stirred for 10 min and equilibrated with 10 mL of water and 40 mL of ethyl acetate. The organic layer was washed with 4×20 mL of water, 10 mL of brine, dried (sodium sulfate) and evaporated. The brown, oily residue was flash chromatographed using 1:19, 1:9, and 1:4 ethyl acetate-hexane as stepwise gradient. The main band with Rf 0.16 (TLC 1:4 acetate-hexane) was evaporated to give 1,3-di-O-acetyl-1,25-dihydroxy-23-yne-cholecalciferol (22) a colorless foam, 0.0939 g.

### Synthetic Example 20

### Synthesis of (3aR,4S,7aR)-7a-Methyl-1-[1-(4-hydroxy-4-methyl-pent-2-ynyl)-cyclopropyl]-3a,4,5,6,7,7a-hexahydro-3H-inden-4-ol

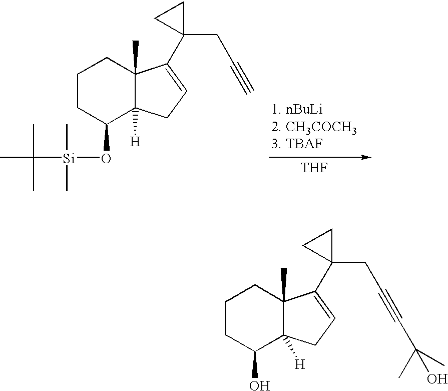

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])cyclopropyl}ethynyl (1.0 g, 2.90 mmol) in tetrahydrofurane (15 mL) at −78° C. was added n-BuLi (2.72 mL, 4.35 mmol, 1.6M in hexane). After stirring at −78° C. for 1 h., acetone (2.5 mL, 34.6 mmol) was added and the stirring was continued for 2.5 h. NH₄Cl_{aq} was added (15 mL) and the mixture was stirred for 15 min at room temperature then extracted with AcOEt (2×50 mL). The combined extracts were washed with brine (50 mL) and dried over Na₂SO₄. The residue after evaporation of the solvent (2.4 g) was purified by FC (50 g, 10% AcOEt in hexane) to give (3aR,4S,7aR)-5-{1-[4-(tert-Butyl-dimethyl-silanyloxy)-7a-methyl-3a,4,5,6,7,7a-hexahydro-3H-inden-1-yl]-cyclopropyl}-2-methyl-pent-3-yn-2-ol (1.05 g, 2.61 mmol) which was treated with tetrabutylammonium fluoride (6 mL, 6 mmol, 1.0M in THF) and stirred at 65-75° C. for 48 h. The mixture was diluted with AcOEt (25 mL) and washed with water (5×25 mL), brine (25 mL). The combined aqueous washes were extracted with AcOEt (25 mL) and the combined organic extracts were dried over Na₂SO₄. The residue after evaporation of the solvent (1.1 g) was purified by FC (50 g, 20% AcOEt in hexane) to give the titled compound (0.75 g, 2.59 mmol, 90%). [α]³⁰_{D}=+2.7 c 0.75, CHCl₃. ¹H NMR (CDCl₃): 5.50 (1H, m), 4.18 (1H, m), 2.40 (2H, s), 2.35-1.16 (11H, m), 1.48 (6H, s), 1.20 (3H, s), 0.76-0.50 (4H, m); ¹³C NMR (CDCl₃): 156.39, 125.26, 86.39, 80.19, 69.21, 65.16, 55.14, 46.94, 35.79, 33.60, 31.67, 29.91, 27.22, 19.32, 19.19, 17.73, 10.94, 10.37; MS HREI Calculated for C₂₂H₂₈O₂ M+288.2089, Observed M+ 288.2091.

### Synthetic Example 21

### Synthesis of (3aR,4S,7aR)-7a-Methyl-1-[1-(4-hydroxy-4-methyl-pent-2Z-enyl)-cyclopropyl]-3a,4,5,6,7,7a-hexahydro-3H-inden-4-ol

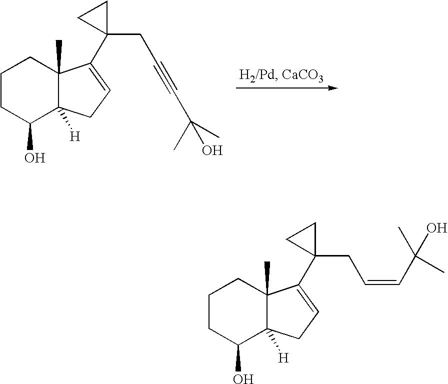

The mixture of (3aR,4S,7aR)-7a-Methyl-1-[1-(4-hydroxy-4-methyl-pent-2-ynyl)-cyclopropyl]-3a,4,5,6,7,7a-hexahydro-3H-inden-4-ol (0.72 g, 2.50 mmol), ethyl acetate (10 mL), hexane (24 mL), absolute ethanol (0.9 mL), quinoline (47 L) and Lindlar catalyst (156 mg, 5% Pd on CaCO₃) was hydrogenated at room temperature for 2 h. The reaction mixture was filtered through a celite pad and the pad was washed with AcOEt. The filtrates and the washes were combined and washed with 1M HCl, NaHCO₃ and brine. After drying over Na₂SO₄ the solvent was evaporated and the residue (0.79 g) was purified by FC (45 g, 20% AcOEt in hexane) to give the titled compound (640 mg, 2.2 mmol, 88%).

### Synthetic Example 22

### Synthesis of (3aR,4S,7aR)-7a-Methyl-1-[1-(4-hydroxy-4-methyl-pentyl)-cyclopropyl]-3a,4,5,6,7,7a-hexahydro-3H-inden-4-ol

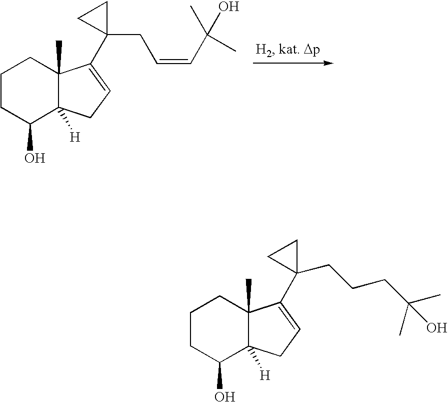

The mixture of (3aR,4S,7aR)-7a-Methyl-1-[1-(4-hydroxy-4-methyl-pent-2Z-enylycyclopropyl]-3a,4,5,6,7,7a-hexahydro-3H-inden-4-ol (100 mg, 0.34 mmol), 1,4-bis(diphenyl-phosphino)butane 1,5 cyclooctadiene rhodium tetrafluoroborate (25 mg, 0.034 mmol), dichloromethane (5 mL) and one drop of mercury was hydrogenated using Paar apparatus at room temperature and 50 p.s.i. pressure for 3 h. The reaction mixture was filtered through Celite pad, which was then washed with ethyl acetate. The combine filtrates and washes were evaporated to dryness (110 mg) and purified by FC (10 g, 20% AcOEt in hexane) to give the titled compound (75 mg, 0.26 mmol, 75%). [α]³⁰_{D}=−8.5 c 0.65, CHCl₃. ¹H NMR (CDCl₃): 5.37 (1H, m,), 4.14 (1H, m), 2.37-1.16 (17H, m), 1.19 (6H, s), 1.18 (3H, s), 0.66-0.24 (4H, m); MS HREI Calculated for C₁₉H₃₂O₂ M+H 292.2402, Observed M+H 292.2404.

### Synthetic Example 23

### Synthesis of (3aR,7aR)-7a-Methyl-1-[1-(4-methyl-4-trimethylsilanyloxy-pentyl)-cyclopropyl]-3a,4,5,6,7,7a-hexahydro-3H-inden-4-one

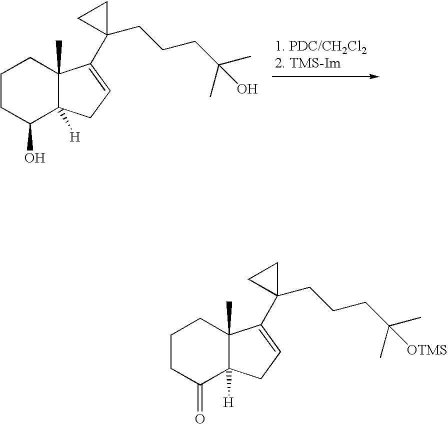

To a stirred suspension of (3aR,4S,7aR)-7a-Methyl-1-[1-(4-hydroxy-4-methyl-pentenyl)-cyclopropyl]-3a,4,5,6,7,7a-hexahydro-3H-inden-4-ol (440 mg, 1.50 mmol) and Celite (2.0 g) in dichloromethane (10 mL) at room temperature wad added pyridinium dichromate (1.13 g, 3.0 mmol). The resulting mixture was stirred for 5 h filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pentenyl) cyclopropyl]-3a,4,5,6,7,7a-hexahydro-3H-inden-4-one (426 mg, 1.47 mmol, 98%). To a stirred solution of (3aR,7aR)-7a-Methyl-1-[1-(4-hydroxy methyl-pentenyl)-cyclopropyl]-3a,4,5,6,7,7a-hexahydro-3H-inden-4-one (424 mg, 1.47 mmol) in dichloromethane (10 mL) at room temperature was added trimethylsilyl-imidazole (0.44 mL, 3.0 mmol). The resulting mixture was stirred for 1.0 h filtered through silica gel (10 g) and the silica gel pad was washed with 10% AcOEt in hexane. Combined filtered and washes were evaporated to give the titled compound (460 mg, 1.27 mmol, 86%). [α]²⁹_{D}=−9.9 c 0.55, CHCl₃. ¹H NMR (CDCl₃): 5.33 (1H, dd, J=3.2, 1.5 Hz), 2.81 (1H, dd, J=10.7, 6.2 Hz), 2.44 (1H, ddd, J=15.6, 10.7, 1.5 Hz), 2.30-1.15 (13H, m) overlapping 2.03 (ddd, J=15.8, 6.4, 3.2 Hz), 1.18 (6H, s), 0.92 (3H, s), 0.66-0.28 (4H, m), 0.08 (9H, s); ¹³C NMR (CDCl₃): 211.08 (0), 155.32 (0), 124.77(1), 73.98 (0), 64.32 (1), 53.91 (0), 44.70 (2), 40.45 (2), 38.12 (2), 34.70 (2), 29.86 (3), 29.80 (3), 26.80 (2), 24.07 (2), 22.28 (2), 21.24 (0), 18.35 (3), 12.60 (2), 10.64 (2), 2.63 (3); MS HRES Calculated for C₂₂H₃₈O₂Si M+ 362.2641. Observed M+ 362.2648.

### Synthetic Example 24

### Synthesis of (3aR,7aR)-7a-Methyl-1-[1-(4-methyl-4-trimethylsilanyloxy-pent-2-ynyl)-cyclopropyl]-3a,4,5,6,7,7a-hexahydro-3H-inden-4-one

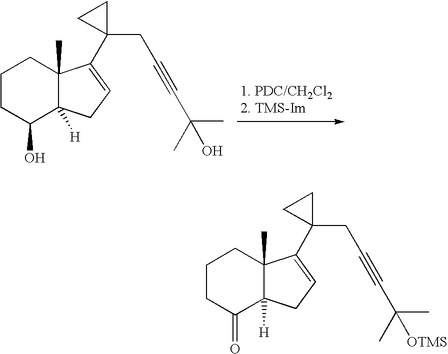

To a stirred suspension of (3aR,4S,7aR)-7a-Methyl-1-[1-(4-hydroxy-4-methyl-pent-2-ynyll)-cyclopropyl]-3a,4,5,6,7,7a-hexahydro-3H-inden-4-ol (381 mg, 1.32 mmol) and Celite (2.0 g) in dichloromethane (10 mL) at room temperature wad added pyridinium dichromate (1.0 g, 2.65 mmol). The resulting mixture was stirred for 1.5 h filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pent-2-ynyll)-cyclopropyl]-3a,4,5,6,7,7a-hexahydro-3H-inden-4-one (360 mg, 1.26 mmol, 95%). To a stirred solution of (3aR,7aR)-7a-Methyl-1-[1-(4-hydroxy-4-methyl-pent-2-ynyll)cyclopropyl]-3a,4,5,6,7,7a-hexahydro-3H-inden-4-one (360 mg, 1.26 mmol) in dichloromethane (10 mL) at room temperature was added trimethylsilyl-imidazole (0.25 mL, 1.7 mmol). The resulting mixture was stirred for 0.5 h filtered through silica gel (10 g) and the silica gel pad was washed with 5% AcOEt in hexane. Combined filtered and washes were evaporated to give the titled compound (382 mg, 1.07 mmol, 81%).

### Synthetic Example 25

### Synthesis of 1-alpha,25-Dihydroxy-16-ene-20-cyclopropyl-23,24-yne-cholecalciferol (23)

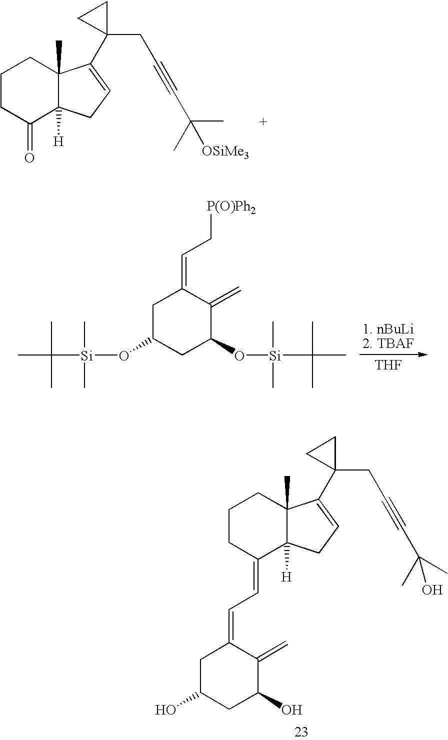

To a stirred solution of a (1S,5R)-1,5-bis-((tert-butyldimethyl)silanyloxy)-3-[2-(diphenylphosphinoyl)eth-(Z)-ylidene]-2-methylene-cyclohexane (513 mg, 0.88 mmol) in tetrahydrofurane (6 mL) at −78° C. was added n-BuLi (0.55 mL, 0.88 mmol). The resulting mixture was stirred for 15 min and solution of (3aR,7aR)-7a-Methyl-1-[1-(4-methyl-4-trimethylsilanyloxy-pent-2-ynyll)-cyclopropyl]-3a,4,5,6,7,7a-hexahydro-3H-inden-4-one (179 mg, 0.50 mmol, in tetrahydrofurane (2 mL) was added dropwise. The reaction mixture was stirred at −72° C. for 3.5 h diluted with hexane (25 mL) washed brine (30 mL) and dried over Na₂SO₄. The residue (716 mg) after evaporation of the solvent was purified by FC (15 g, 5% AcOEt in hexane) to give 1-alpha,3-beta-Di(tert-Butyl-dimethyl-silanyloxy)-25-trimethylsilanyloxy-16-ene-20-cyclopropyl-23,24-yne-cholecalciferol (324 mg, 045 mmol). To the 1-alpha,3-beta-Di(tert-Butyl-dimethyl-silanyloxy)-25-trimethylsilanyloxy-16-ene-20-cyclopropyl-23,24-yne-cholecalciferol (322 mg, 0.45 mmol) tetrabutylammonium fluoride (4 mL, 4 mmol, 1M solution in THF) was added, at room temperature. The mixture was stirred for 18 h diluted with AcOEt (25 mL) and washed with water (5×20 mL), brine (20 mL) and dried over Na₂SO₄. The residue (280 mg) after evaporation of the solvent was purified by FC (10 g, 50% AcOEt in hexane and AcOEt) to give the titled compound (23) (172 mg, 0.41 mmol, 82%). [α]³¹_{D}=+ 32.4 c 0.50, MeOH. UV λmax (EtOH): 261 nm (ε 11930); ¹H NMR (CDCl₃): 6.36 (1H, d, J=11.3 Hz), 6.09 (1H, d, J=11.3 Hz), 5.45 (1H, m), 5.33 (1H, m), 5.01 (1H, s), 4.45 (1H, m), 4.22 (1H, m), 2.80 (1H, m), 2.60 (1H, m), 2.50-1.10 (16H, m), 1.45 (6H, s), 0.81 (3H, s), 0.72-0.50 (4H, m); MS HRES Calculated for C₂₈H₃₈O₃ M+ 422.2821, Observed M+ 422.2854.

### Synthetic Example 26

### Synthesis of 1-alpha,25-Dihydroxy-16-ene-20-cyclopropyl-23,24-yne-19-nor-cholecalciferol (24)

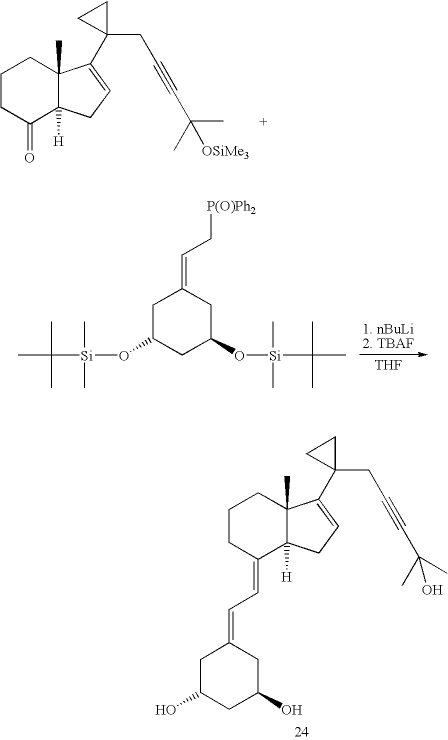

To a stirred solution of a (1R,3R)-1,3-bis-((tert-butyldimethyl)silanyloxy)-5-[2-(diphenylphosphinoyl)ethylidene]-cyclohexane (674 mg, 1.18 mmol) in tetrahydrofurane (8 mL) at −78° C. was added n-BuLi (0.74 mL, 1.18 mmol). The resulting mixture was stirred for 15 min and solution of (3aR,7aR)-7a-Methyl-1-[1-(4-methyl-4-trimethylsilanyloxy-pent-2-ynyl) cyclopropyl]-3a,4,5,6,7,7a-hexahydro-3H-inden-4-one (235 mg, 0.66 mmol, in tetrahydrofurane (3 mL) was added dropwise. The reaction mixture was stirred at −72° C. for 3.5 h diluted with hexane (25 mL) washed brine (30 mL) and dried over Na₂SO₄. The residue (850 mg) after evaporation of the solvent was purified by FC (159, 5% AcOEt in hexane) to give 1-alpha,3-beta-Di(tert-Butyl-dimethyl-silanyloxy)-25-trimethylsilanyloxy-16-ene-20-cyclopropyl-23,24-yne-19-nor-cholecalciferol (330 mg, 0.46 mmol). To the 1-alpha,3-beta-7-Di(tert-Butyl-dimethyl-silanyloxy)-25-trimethylsilanyloxy-16-ene-20-cyclopropyl-23,24-yne-19-nor-cholecalciferol (328 mg, 0.46 mmol) tetrabutylammonium fluoride (5 mL, 5 mmol, 1M solution in THF) was added, at room temperature. The mixture was stirred for 62 h diluted with AcOEt (25 mL) and washed with water (5×20 mL), brine (20 mL) and dried over Na₂SO₄. The residue (410 mg) after evaporation of the solvent was purified by FC (10 g, 50% AcOEt in hexane and AcOEt) to give the titled compound (24) (183 mg, 0.45 mmol, 68%). [[α]²⁹_{D}=+ 72.1 c 0.58, MeOH. UV λmax (EtOH): 242 nm (ε 29286), 251 nm (ε 34518), 260 nm (ε2357R(CDCl₃): 6.30 (1H, d, J=11.3 Hz), 5.94 (1H, d, J=11.3 Hz), 5.48 (1H, m), 4.14(1H, m), 4.07(1H, m), 2.78(2H, m), 2.52-1.10 (18H, m), 1.49 (6H, s), 0.81 (3H, s), 0.72-0.50 (4H, m); MS HRES Calculated for C₂₇H₃₈O₃ M+ 410.2821, Observed M+ 410.2823.

### Synthetic Example 27

### Synthesis of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol

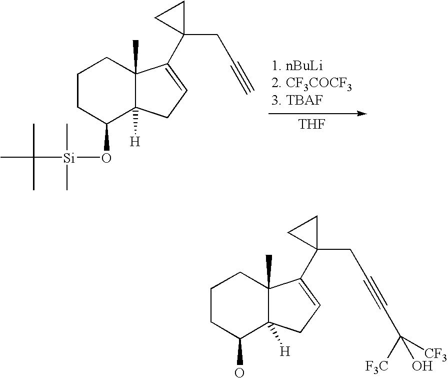

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])cyclopropyl}-ethynyl (1.95 g, 5.66 mmol) in tetrahydrofurane (35 mL) at −78° C. was added n-BuLi (4.3 mL, 6.88 mmol, 1.6M in hexane). After stirring at −78° C. for 1 h., hexafluoroacetone (six drops from the cooling finger) was added and the stirring was continued for 1 h. NH₄Cl_{aq} was added (10 mL) and the mixture was allowed to warm to room temperature. The reaction mixture was diluted with brine (100 mL) and extracted with hexane (2×125 mL). The combined extracts were dried over Na₂SO₄. The residue after evaporation of the solvent (8.2 g) was purified by FC (150 g, 10% AcOEt in hexane) to give (3aR,4S,7aR)-5-{1-[4-(tert-Butyl-dimethyl-silanyloxy)-7a-methyl-3a,4,5,6,7,7a-hexahydro-3H-inden-1-yl]-cyclopropyl}-1,1,1-trifluoro-2-trifluoromethyl-pent-3-yn-2-ol (2.73 g, 5.35 mmol) which was treated with tetrabutylammonium fluoride (20 mL, 20 mmol, 1.0M in THF) and stirred at 65-75° C. for 30 h. The mixture was diluted with AcOEt (150 mL) and washed with water (5×150 mL), brine (150 mL). The combined aqueous washes were extracted with AcOEt (150 mL) and the combined organic extracts were dried over Na₂SO₄. The residue after evaporation of the solvent (3.2 g) was purified by FC (150 g, 20% AcOEt in hexane) to give the titled compound (2.05 g, 5.17 mmol, 97%). [α]²⁸_{D}=+ 6.0 c 0.47, CHCl₃. ¹H NMR (CDCl₃): 5.50 (1H, br. s), 4.16 (1H, br. s), 3.91 (1H, s), 2.48 (1H, part A of the AB quartet, J=17.5 Hz), 2.43 (1H, part B of the AB quartet, J=17.5 Hz), 2.27 (1H, m), 2.00-1.40 (9H, m), 1.18 (3H, s), 0.8-0.5 (4H, m); ¹³C NMR (CDCl₃): 155.26(0), 126.68 (1), 121.32 (0, q, J=284 Hz), 90.24 (0), 71.44 (0, sep. J=34 Hz), 70.54 (0), 69.57 (1), 55.17 (1), 47.17 (0), 36.05 (2), 33.63 (2), 30.10 (2), 27.94 (2), 19.50 (3), 19.27 (0), 17.90 (2), 11.56 (2), 11.21 (2); MS HREI Calculated for C₁₉H₂₂O₂F₆ M+ 396.1524, Observed M+ 396.1513.

### Synthetic Example 28

### Synthesis of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-H-inden-4-one

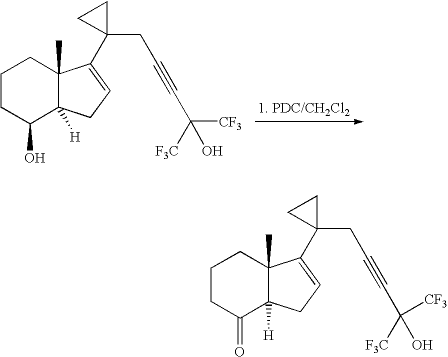

To a stirred suspension of (3aR,4S,7aR)-7a-Methyl-1-[1-(5,5,5-trifluoro-4-hydroxy-4-trifluoromethyl-pent-2-ynyl)-yclopropyl]-3a,4,5,6,7,7a-hexahydro-3H-inden-4-ol (504 mg, 1.27 mmol) and Celite (1.5 g) in dichloromethane (12 mL) at room temperature wad added pyridinium dichromate (0.98 g, 2.6 mmol). The resulting mixture was stirred for 2.5 h filtered through silica gel (5 g), and then silica gel pad was washed with 20% AcOEt in hexane. The combined filtrate and washes were evaporated, to give a titled compound (424 mg, 1.08 mmol, 85%). [α]²⁸_{D}=+3.1 c 0.55, CHCl₃. ¹H NMR (CDCl₃): 5.46 (1H, br. s), 3.537 (1H, s), 2.81 (1H, dd, J=10.7, 6.5 Hz), 2.49-1.76 (10H, m), 0.90 (3H, s), 0.77-0.53 (4H, m); MS HREI Calculated for C₁₉H₂₀O₂F₆ M+H 395.1440, Observed M+H 395.1443.

### Synthetic Example 29

### Synthesis of 1-alpha,25-Dihydroxy-16-ene-20-cyclopropyl-23,24-yne-26,27-hexafluoro-19-nor-cholecalciferol (25)

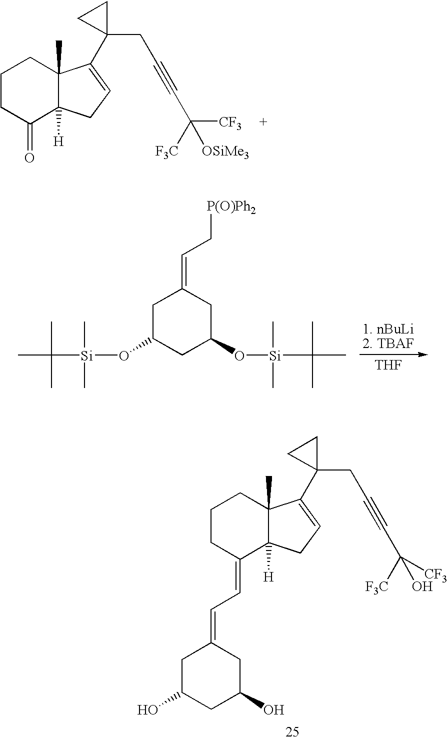

To a stirred solution of a (1R,3R)-1,3-bis-((tert-butyldimethyl)silanyloxy)-5-[2-(diphenylphosphinoyl)ethylidene]-cyclohexane (900 mg, 1.58 mmol) in tetrahydrofurane (8 mL) at −78° C. was added n-BuLi (1.0 mL, 1.6 mmol). The resulting mixture was stirred for 15 min and solution of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one (200 mg, 0.51 mmol, in tetrahydrofurane (3 mL) was added dropwise. The reaction mixture was stirred at −72° C. for 3.5 h diluted with hexane (25 mL) washed brine (30 mL) and dried over Na₂SO₄. The residue (850 mg) after evaporation of the solvent was purified by FC (20 g, 10% AcOEt in hexane) to give 1-alpha,3-beta-Di(tert-Butyl-dimethyl-silanyloxy)-25-hydroxy-16-ene-20-cyclopropyl-23,24-yne-26,27-hexafluoro-19-nor-cholecalciferol (327 mg, 0.44 mmol, 86%). To the 1-alpha,3-beta-Di(tert-Butyl-dimethyl-silanyloxy)-25-hydroxy-16-ene-20-cyclopropyl-23,24-yne-26,27-hexafluoro-19-nor-cholecalciferol (327 mg, 0.44 mmol). Tetrabutylammonium fluoride (4 mL, 4 mmol, 1M solution in THF) was added, at room temperature. The mixture was stirred for 24 h. diluted with AcOEt (25 mL) and washed with water (5×20 mL), brine (20 mL) and dried over Na₂SO₄. The residue (250 mg) after evaporation of the solvent was purified by FC (10 g, 50% AcOEt in hexane and AcOEt) to give the titled compound (25) (183 mg, 0.45 mmol, 68%). [α]³⁰_{D}=+ 73.3 c 0.51, EtOH. UV λmax (EtOH): 243 nm (ε29384.), 251 nm (ε34973), 260 nm (ε23924); NMR (CDCl₃): 6.29 (1H, d, J=11.1 Hz), 5.93 (1H, d, J=11.1 Hz), 5.50 (1H, m), 4.12 (1H, m), 4.05 (1H, m), 2.76 (2H, m), 2.55-1.52 (18H, m), 0.80 (3H, s), 0.80-0.49 (4H, m); ¹³C NMR (CDCl₃): 155.24(0), 141.78 (0), 131.28 (0), 126.23 (1), 123.65 (1), 121.09 (0, q, J=285 Hz), 115.67 (1), 89.63 (0), 70.42 (0), 67.48 (1), 67.29 (1), 59.19 (1), 49.87 (0), 44.49 (2), 41.98 (2), 37.14 (2), 35.76 (2), 29.22 (2), 28.47 (2), 27.57 (2), 23.46 (2), 19.32 (0), 17.97 (3), 11.89 (2), 10.18 (2); MS HRES Calculated for C₂₇H₃₂O₃F₆ M+H 519.2329. Observed M+H 519.2325.

### Synthetic Example 30

### Synthesis of 1-alpha,25-Dihydroxy-16-ene-20-cyclopropyl-23,24-yne-26,27 hexafluoro-cholecalciferol (26)

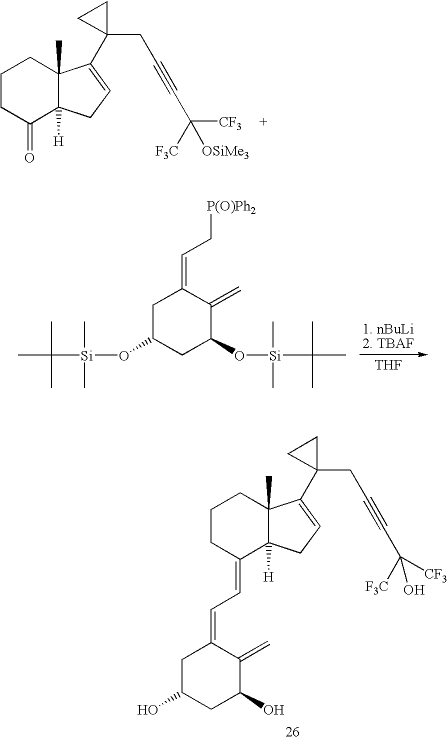

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (921 mg, 1.58 mmol) in tetrahydrofurane (8 mL) at −78° C. was added n-BuLi (1.0 mL, 1.6 mmol). The resulting mixture was stirred for 15 min and solution of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one (197 mg, 0.50 mmol, in tetrahydrofurane (2 mL) was added dropwise. The reaction mixture was stirred at −72° C. for 3.5 h diluted with hexane (25 mL) washed brine (30 mL) and dried over Na₂SO₄. The residue (876 mg) after evaporation of the solvent was purified by FC (209, 105% AcOEt in hexane) to give 1-alpha,3-beta-Di(tert-Butyl-dimethyl-silanyloxy)-25-hydroxy-16-ene-20-cyclopropyl-23,24-yne-26,27-hexafluoro-cholecalciferol (356 mg, 0.47 mmol). To the 1-alpha,3-beta-Di(tert-Butyl-dimethyl-silanyloxy)-25-hydroxy-16-ene-20-cyclopropyl-23,24-yne-26,27-hexafluoro-cholecalciferol (356 mg, 0.47 mmol) tetrabutylammonium fluoride (5 mL, 5 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (270 mg) after evaporation of the solvent was purified by FC (20 g, 50% AcOEt in hexane and AcOEt) to give the titled compound (26)-(216 mg, 0.41 mmol, 87%). [α]³⁰_{D}=+40.0 c 0.53, EtOH. UV λmax (EtOH): 262 nm (ε 12919); ¹H NMR (CDCl₃): 6.38 (1H, d, J=11.5 Hz), 6.10 (1H, d, J=11.1 Hz), 5.49 (1H, m), 5.35 (1H, s), 5.02 (1H, s), 4.45 (1H, m), 4.25 (1H, m), 3.57 (1H, s), 2.83-1.45 (18H, m), 0.82 (3H, s), 0.80-0.51 (4H, m); MS HRES Calculated for C₂₈H₃₂O₃F₆ M+H 531.2329. Observed M+H 531.2337.

### Synthetic Example 31

### Synthesis of (3aR,4S,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-ol

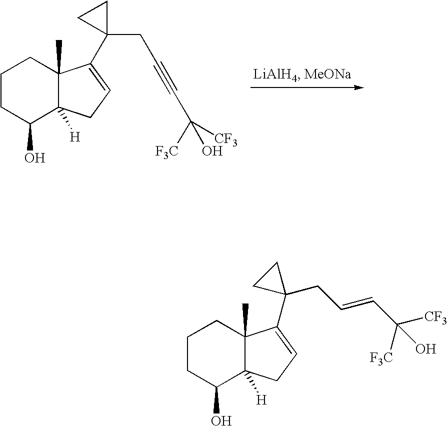

To a lithium aluminum hydride (4.5 mL, 4.5 mmol, 11.0M in THF) at 5° C. was added first solid sodium methoxide (245 mg, 4.6 mmol) and then dropwise solution of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (360 mg, 0.91 mmol) in tetrahydrofurane (5 mL). After addition was completed the mixture was stirred under reflux for 2.5 h. Then it was cooled in the ice-bath and quenched with water (2.0 mL) and sodium hydroxide (2.0 mL, 2.0 M water solution); diluted with ether (50 mL) stirred for 30 min, MgSO₄ (5 g) was than added and stirring was continued for 30 min. The residue after evaporation of the filtrates (0.42 g) was purified by FC (20 g, 20% AcOEt in hexane) to give the titled compound (315 mg, 0.79 mmol, 87%). [α]²⁸_{D}=+2.0 c 0.41, CHCl₃. ¹H NMR (CDCl₃): 6.24 (1H, dt, J=15.7, 6.7 Hz), 5.60 (1H, d, J=15.7 Hz), 5.38 (1H, br. s), 4.13 (1H, br. s), 3.27 (1H, s), 2.32-1.34 (12H, m), 1.15 (3H, s), 0.80-0.45 (4H, m); ¹³C NMR (CDCl₃): 155.89(0), 138.10 (1), 126.21 (1), 122.50 (0, q, J=287 Hz), 119.15 (1), 76.09 (0, sep. J=31 Hz), 69.57 (1), 55.33 (1), 47.30 (0), 40.31 (2), 36.05 (2), 33.71 (2), 30.10 (2), 20.36 (0), 19.46 (3), 17.94 (2), 11.96 (2), 11.46 (2); MS REI Calculated for C₁₉H₂₄O₂F₆ M+ 398.1680. Observed M+ 398.1675.

### Synthetic Example 32

### Synthesis of (3aR,7aR)-7a-Methyl-1-[1-(5,5,5-trifluoro-4-trifluoromethyl-4-trimethylsilanyloxy-pen-2E-enyl)-cyclopropyl]3a,4,5,6,7,7a-hexahydro-3H-inden-4-one

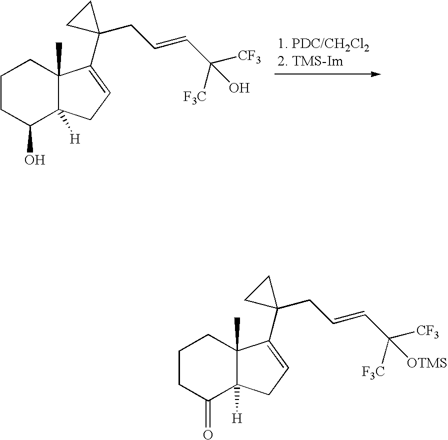

To a stirred suspension of (3aR,4S,7aR)-7a-Methyl-1-[1-(5,5,5-trifluoro-4-hydroxy-4-trifluoromethyl-pent-2E-enylcyclopropyl]-3a,4,5,6,7,7a-hexahydro-3H-inden-4-ol (600 mg, 1.51 mmol) and Celite (2.0 g) in dichloromethane (10 mL) at room temperature wad added pyridinium dichromate (1.13 g, 3.0 mmol). The resulting mixture was stirred for 3.5 h filtered through silica gel (10 g), and then silica gel pad was washed with 25% AcOEt in hexane. The combined filtrate and washes were evaporated, to give a crude (3aR,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-one (550 mg, 1.39 mmol, 92%). To a stirred solution of (3aR,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-one (550 mg, 1.39 mmol) in dichloromethane (15 mL) at room temperature was added trimethylsilyl-imidazole (1.76 mL, 12.0 mmol). The resulting mixture was stirred for 1.0 h filtered through silica gel (10 g) and the silica gel pad was washed with 10% AcOEt in hexane. Combined filtered and washes were evaporated to give the titled compound (623 mg, 1.33 mmol, 88%). [α]²⁸_{D}=−1.6 c 0.51, CHCl₃. ¹H NMR (CDCl₃): 6.14 (1H, dt, J=15.5, 6.7 Hz), 5.55 (1H, d, J=115.5 Hz), 5.35 (1H, m), 2.80 (1H, dd, J=10.7, 6.4 Hz), 2.47-1.74 (10H, m), 0.90 (3H, s), 0.76-0.40 (4H, m), 0.2 (9H, s); ¹³C NMR (CDCl₃): 210.99 (0), 154.28(0), 137.41 (1), 126.26 (1), 122.59 (0, q, J=289 Hz), 120.89 (1), 64.31 (1), 53.96 (0), 40.60 (2), 40.13 (2), 35.00 (2), 27.03 (2), 24.21 (2), 20.57 (0), 18.53 (3), 12.41 (2), 10.79 (2), 1.65 (3); MS HRES Calculated for C₂₂H₃₀O₂F₆Si M+H 469.1992. Observed M+H 469.1995.

### Synthetic Example 33

### Synthesis of 1-alpha,25-Dihydroxy-16-ene-20-cyclopropyl-23,24-E-ene-26,27-hexafluoro-19-nor-cholecalciferol (27)

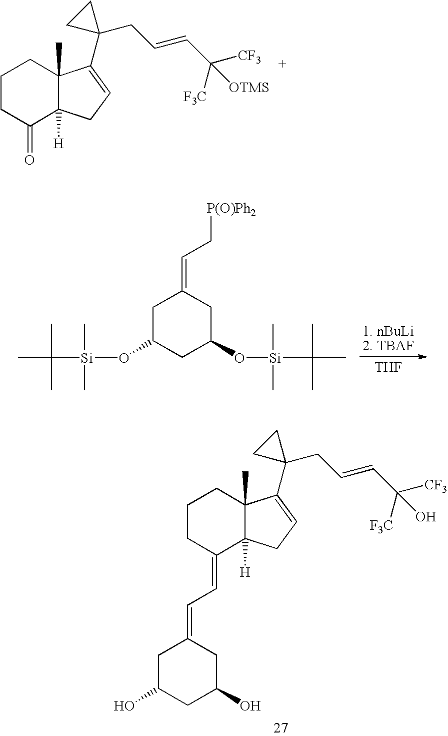

To a stirred solution of a (1R,3R)-1,3-bis-((tert-butyldimethyl)silanyloxy)-5-[2-(diphenylphosphinoyl)ethylidene]-cyclohexane (514 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2E-enylycyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (750 mg) after evaporation of the solvent was purified by FC (15 g, 5% AcOEt in hexane) to give a mixture of 1-alpha,3-beta-Di(tert-Butyl-dimethyl-silanyloxy)-25-trimethylsilanyloxy-16-ene-20-cyclopropyl-23,24-E-ene-26,27-hexafluoro-19-nor-cholecalciferol and 1-alpha,3-beta-Di(tert-Butyl-dimethyl-silanyloxy)-25-hydroxy-16-ene-20-cyclopropyl-23,24-E-ene-26,27-hexafluoro-19-nor-cholecalciferol (250 mg). To the mixture of 1-alpha,3-beta-Di(tert-Butyl-dimethyl-silanyloxy)-25-trimethylsilanyloxy-16-ene-20-cyclopropyl-23,24-E-ene-26,27-hexafluoro-19-nor-cholecalciferol and 1-alpha,3-beta-Di(tert-Butyl-dimethyl-silanyloxy)-25-hydroxy-16-ene-20-cyclopropyl-23,24-E-ene-26,27-hexafluoro-19-nor-cholecalciferol (250 mg) tetrabutylammonium fluoride (4 mL, 4 mmol, 1M solution in THF) was added, at room temperature. The mixture was stirred for 24 h. diluted with AcOEt (25 mL) and washed with water (5×20 mL), brine (20 mL) and dried over Na₂SO₄. The residue (270 mg) after evaporation of the solvent was purified by FC (10 g, 50% AcOEt in hexane and AcOEt) to give the titled compound (27) (157 mg, 0.30 mmol, 70%). [α]³⁰_{D}=+63.3 c 0.45, EtOH. UV λmax (EtOH): 243 nm (ε 30821), 251 nm (ε 36064), 260 nm (ε 24678); ¹H NMR (CDCl₃): 6.29 (1H, d, J=11.3 Hz), 6.24 (1H, dt, J=15.9, 6.4 Hz), 5.92 (1H, d, J=11.1 Hz), 5.61 (1H, d, J=15.7 Hz), 5.38 (1H, m), 4.13 (1H, m), 4.05 (1H, m), 2.88 (1H, s), 2.82-1.34 (19H, m), 0.770 (3H, s), 0.80-0.36 (4H, m); MS HRES Calculated for C₂₇H₃₄O₃F₆ M+H 521.2485. Observed M+H 521.2489.

### Synthetic Example 34

### Synthesis of 1-alpha,25-Dihydroxy-16-ene-20-cyclopropyl-23,24-E-ene-26,27-hexafluoro-cholecalciferol (28)

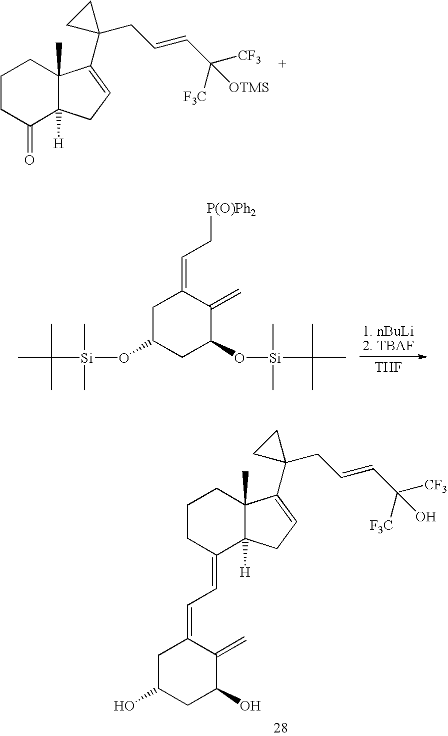

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (525 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2E-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 2.5 h diluted with hexane (35 mL) washed brine (30 mL) and dried over Na₂SO₄. The residue (760 mg) after evaporation of the solvent was purified by FC (15 g, 10% AcOEt in hexane) to give a mixture of 1-alpha,3-beta-Di(tert-Butyl-dimethyl-silanyloxy)-25-trimethylsilanyloxy-16-ene-20-cyclopropyl-23,24-E-ene-26,27-hexafluoro-cholecalciferol and 1-alpha,3-beta-Di(tert-Butyl-dimethyl-silanyloxy)-25-hydroxy-16-ene-20-cyclopropyl-23,24-E-ene-26,27-hexafluoro-cholecalciferol (274 mg). To the mixture of 1-alpha,3-beta-Di(tert-Butyl-dimethyl-silanyloxy)-25-trimethylsilanyloxy-16-ene-20-cyclopropyl-23,24-E-ene-26,27-hexafluoro-cholecalciferol and 1-alpha,3-beta-Di(tert-Butyl-dimethyl-silanyloxy)-25-hydroxy-16-ene-20-cylopropyl-23,24-E-ene-26,27-hexafluoro-cholecalciferol (274 mg) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (280 mg) after evaporation of the solvent was purified by FC (15 g, 50% AcOEt in hexane and AcOEt) to give the titled compound (28) (167 mg, 0.31 mmol, 73%). [α]³⁰_{D}=+ 18.3 c 0.41, EtOH. UV λmax (EtOH): 207 nm (ε 17778), 264 nm (ε 15767); ¹H NMR (CDCl₃): 6.36 (1H, d, J=11.1 Hz), 6.24 (1H, dt, J=15.7, 6.7 Hz), 6.07 (1H, d, J=11.3 Hz), 5.60 (1H, d, J=15.5 Hz), 5.35 (1H, m), 5.33 (1H, s), 5.00 (1H, s), 4.44 (1H, m), 4.23 (1H, m), 3.14 (1H, s), 2.80 (1H, m), 2.60 (1H, m), 2.40-1.40 (15H, m), 0.77 (3H, s), 0.80-0.36 (4H, m); MS HRES Calculated for C₂₈H₃₄O₃F₆ M+H 533.2485. Observed M+H 533.2483.

### Synthetic Example 35

### Synthesis of (3aR,4S,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-ol

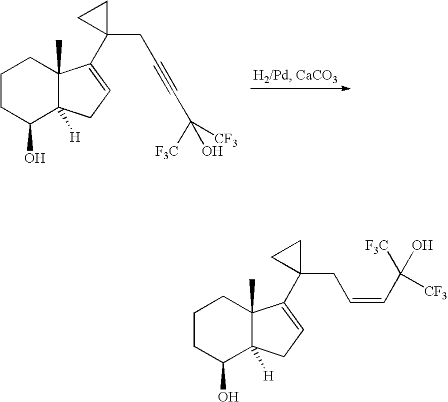

The mixture of (3aR,4S,7aR)-7a-Methyl-1-[1-(5,5,5-trifluoro-4-hydroxy-4-trifluoromethyl-pent-2-ynyl)cyclopropyl]-3a,4,5,6,7,7a-hexahydro-3H-inden-4-ol (300 mg, 0.76 mmol), ethyl acetate (5 mL), hexane (12 mL), absolute ethanol (0.5 mL) quinoline (30 uL) and Lindlar catalyst (75 mg, 5% Pd on CaCO₃) was hydrogenated at room temperature for 2 h. The reaction mixture was filtered through a celite pad and the pad was washed with AcOEt. The solvent was evaporated to give the titled compound (257 mg, 0.65 mmol, 87%). [α]²⁸_{D}=+ 1.8 c 0.61, CHCl₃. ¹H NMR (CDCl₃): 6.08 (1H, dt, J=12.3, 6.7 Hz), 5.47 (1H, m,), 5.39 (1H, d, J=12.1 Hz), 4.15 (1H, br. s), 3.28 (1H, s), 2.52-1.34 (12H, m), 1.16 (3H, s), 0.78-0.36 (4H, m); ¹³C NMR (CDCl₃): 156.66(0), 141.77 (1), 126.51 (1), 122.79 (0, q, J=285 Hz), 115.77 (1), 69.59 (1), 55.41 (1), 47.28 (0), 36.44 (2), 35.90 (2), 33.75 (2), 30.22 (2), 20.89 (0), 19.41 (3), 17.94 (2), 12.05 (2), 11.11 (2); MS HRES Calculated for C₁₉H₂₄O₂F₆ M+H 399.1753. Observed M+H 399.1757.

### Synthetic Example 36

### Synthesis of (3aR,7aR)-7a-Methyl-1-[1-(5,5,5-trifluoro-4-trifluoromethyl-4-trimethylsilanyloxy-pen-2Z-enyl)-cyclopropyl]-3a,4,5,6,7,7a-hexahydro-3H-inden-4-one

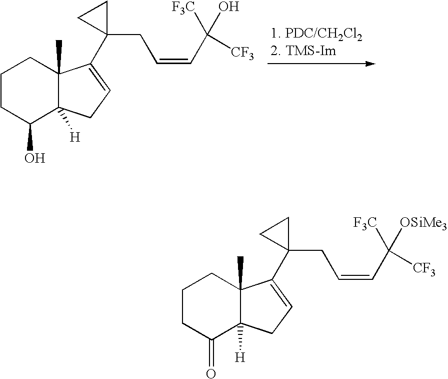

To a stirred suspension of (3aR,4S,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-ol (617 mg, 1.55 mmol) and Celite (2.0 g) in dichloromethane (10 mL) at room temperature wad added pyridinium dichromate (1.17 g, 3.1 mmol). The resulting mixture was stirred for 2.5 h filtered through silica gel (5 g), and then silica gel pad was washed with 20% AcOEt in hexane. The combined filtrate and washes were evaporated, to give a crude (3aR,7aR)-7a-Methyl-1-[1-(5,5,5-trifluoro-4-hydroxy-4-trifluoromethyl-pentenyl)-cylopropyl]-3a,4,5,6,7,7a-hexahydro-3H-inden-4-one (600 mg, 1.51 mmol, 98%). To a stirred solution of (3aR,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-one (600 mg, 1.51 mmol) in dichloromethane (15 mL) at room temperature was added trimethylsilyl-imidazole (1.76 mL, 12.0 mmol). The resulting mixture was stirred for 1.0 h filtered through silica gel (10 g) and the silica gel pad was washed with 10% AcOEt in hexane. Combined filtered and washes were evaporated to give the titled compound (640 mg, 1.37 mmol, 88%). [α]²⁸_{D}=−0.2 c 0.55, CHCl₃. ¹H NMR (CDCl₃): 5.97 (1H, dt, J=12.2, 6.2 Hz), 5.40 (1H, m), 5.38 (1H, d, J=12.2 Hz), 2.82 (1H, dd, J=10.7, 6.6 Hz), 2.60-1.74 (10H, m), 0.89 (3H, s), 0.75-0.36 (4H, m), 0.21 (9H, s); ¹³C NMR (CDCl₃): 210.56 (0), 154.30(0), 139.28(1), 125.81 (1), 122.52 (0, q, J=289 Hz), 118.17 (1), 64.11 (1), 53.69 (0), 40.43 (2), 35.51 (2), 34.85 (2), 26.94 (2), 24.07 (2), 20.89 (0), 18.39 (3), 12.26 (2), 10.61 (2), 1.43 (3); MS HRES Calculated for C₂₂H₃₀O₂F₆Si M+H 469.1992. Observed M+H 469.1992.

### Synthetic Example 37

### Synthesis of 1-alpha,25-Dihydroxy-16-ene-20-cyclopropyl-23,24-Z-ene-26,27-hexafluoro-19-nor-cholecalciferol (29)

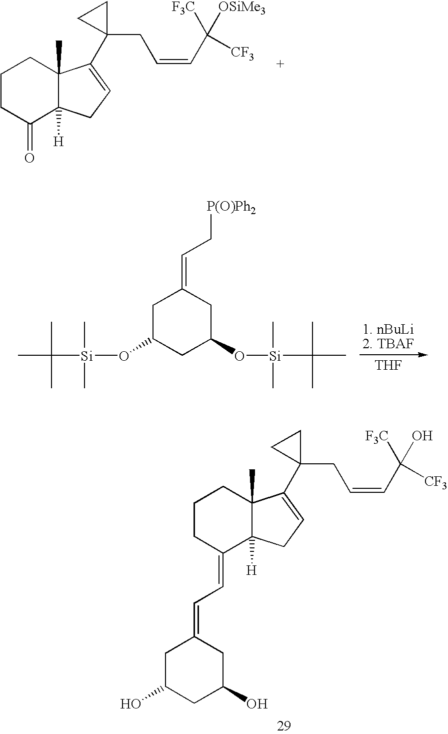

To a stirred solution of a (1R,3R)-1,3-bis-((tert-butyldimethyl)silanyloxy)-5-[2-(diphenylphosphinoyl)ethylidene]-cyclohexane (514 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2Z-enyl)-cyopropyl]-3a,4,5,6,7,7a-hexahydro-3H-inden-4-one (194 mg, 0.41 mmol, in tetrahydrofurane (2 mL) was added dropwise. The reaction mixture was stirred at −72° C. for 3.0 h diluted with hexane (35 mL) washed brine (30 mL) and dried over Na₂SO₄. The residue (750 mg) after evaporation of the solvent was purified by FC (15 g, 10% AcOEt in hexane) to give a mixture of 1-alpha,3-beta-Di(tert-Butyl-dimethyl-silanyloxy)-25-trimethylsilanyloxy-16-ene-20-cyclopropyl-23,24-Z-ene-26,27-hexafluoro-19-nor-cholecalciferol and 1-alpha,3-beta-Di(tert-Butyl-dimethyl-silanyloxy)-25-hydroxy-16-ene-20-cyclopropyl-23,24-Z-ene-26,27-hexafluoro-19-nor-cholecalciferol (230 mg). To the mixture of 1-alpha,3-beta-Di(tert-Butyl-dimethyl-silanyloxy)-25-trimethylsilanyloxy-16-ene-20-cyclopropyl-23,24-Z-ene-26,27-hexafluoro-19-nor-cholecalciferol and 1-alpha,3-beta-Di(tert-Butyl-dimethyl-silanyloxy)-25-hydroxy-16-ene-20-cyclopropyl-23,24-Z-ene-26,27-hexafluoro-19-nor-cholecalciferol (230 mg) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (260 mg) after evaporation of the solvent was purified by FC (10 g, 50% AcOEt in hexane and AcOEt) to give the titled compound (29) (1327 mg, 0.25 mmol, 62%). [α]²⁸_{D}=+ 53.6 c 0.33, EtOH. UV λmax (EtOH): 243 nm (ε 26982). 251 nm (ε 32081), 260 nm (ε 21689); ¹H NMR (CDCl₃): 6.29 (1H, d, J=12.7 Hz), 6.08 (1H, dt, J=12.5, 6.7 Hz), 5.93 (1H, d, J=11.1 Hz), 5.46 (1H, m,), 5.40 (1H, d, J=12.7 Hz)), 4.12 (1H, m), 4.05 (1H, m), 3.14 (1H, s), 2.80-1.40 (19H, m), 0.77 (3H, s), 0.80-0.36 (4H, m); MS HRES Calculated for C₂₇H₃₄O₃F₆ M+H 521.2485. Observed M+H 521.2487.

### Synthetic Example 38

### Synthesis of 1-alpha,25-Dihydroxy-16-ene-20-cyclopropyl-23,242-ene-26,27-hexafluoro-cholecalciferol (30)

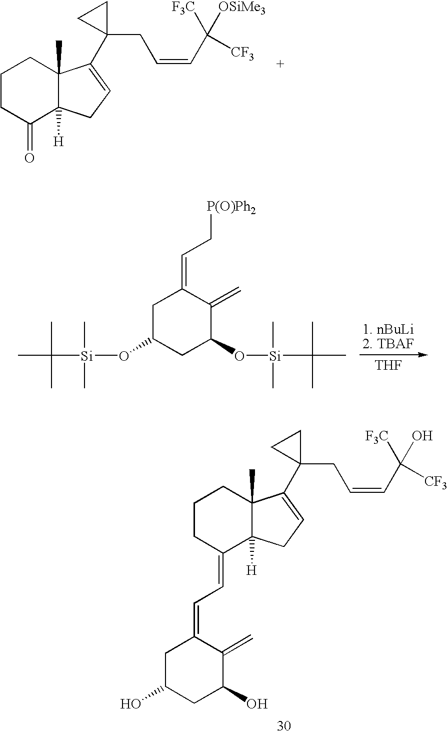

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (525 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2Z-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 2.5 h diluted with hexane (35 mL) washed brine (30 mL) and dried over Na₂SO₄. The residue (680 mg) after evaporation of the solvent was purified by FC (15 g, 10% AcOEt in hexane) to give a mixture of 1-alpha,3-beta-Di(tert-Butyl-dimethyl-silanyloxy)-25-trimethylsilanyloxy-16-ene-20-cyclopropyl-23,24-Z-ene-26,27-hexafluoro-cholecalciferol and 1-alpha,3-beta-Di(tert-Butyl-dimethyl-silanyloxy)-25-hydroxy-16-ene-20-cyclopropyl-23,24-Z-ene-26,27-hexafluoro-cholecalciferol (310 mg). To the mixture of 1-alpha,3-beta-Di(tert-Butyl-dimethyl-silanyloxy)-25-trimethylsilanyloxy-16-ene-20-cyclopropyl-23,24-Z-ene-26,27-hexafluoro-cholecalciferol and 1-alpha,3-beta-Di(tert-Butyl-dimethyl-silanyloxy)-25-hydroxy-16-ene-20-cyclopropyl-23,24-Z-ene-26,27-hexafluoro-cholecalciferol (310 mg) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (370 mg) after evaporation of the solvent was purified by FC (10 g, 50% AcOEt in hexane and AcOEt) to give the titled compound (30) (195 mg, 0.37 mmol, 85%). [α]³⁰_{D}=+ 9.4 c 0.49, EtOH. UV λmax (EtOH): 262 nm (ε 11846); ¹H NMR (CDCl₃): 6.36 (1H, d, J=11.1 Hz), 6.08 (2H, m), 5.44 (1H, m), 5.40 (1H, d, J=12.3 Hz), 5.32 (1H, s), 5.00 (1H, s), 4.43 (1H, m), 4.23 (1H, m), 3.08 (1H, s), 2.80 (1H, m), 2.60 (1H, m), 2.55-1.40 (15H, m), 0.77 (3H, s), 0.80-0.34 (4H, m); MS HRES Calculated for C₂₈H₃₄O₃F₆ M+H 533.2485. Observed M+H 533.2502.

### Synthetic Example 39

### Synthesis of 1-alpha,25-Dihydroxy-16-ene-20-cyclopropyl-19-nor-cholecalciferol (31)

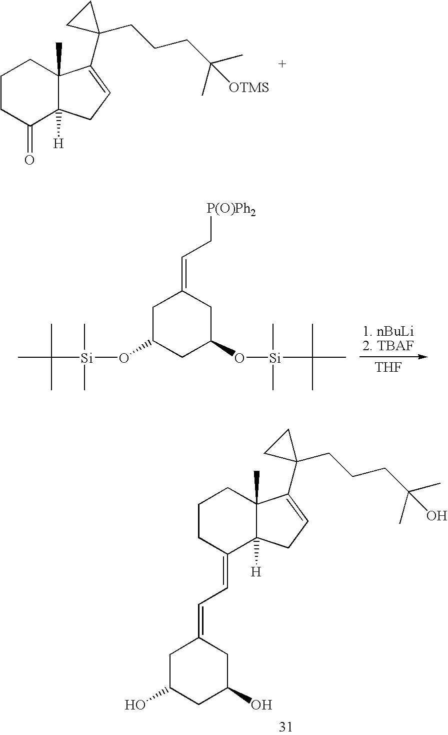

To a stirred solution of a (1R,3R)-1,3-bis-((tert-butyldimethyl)silanyloxy)-5-[2-(diphenylphosphinoyl)ethylidene]-cyclohexane (697 mg, 1.22 mmol) in tetrahydrofurane (9 mL) at −78° C. was added n-BuLi (0.77 mL, 1.23 mmol). The resulting mixture was stirred for 15 min and solution of (3aR,7aR)-7a-Methyl-1-[1-(4-methyl-4-trimethylsilanyloxy-pentyl)-cyclopropyl]-3a,4,5,6,7,7a-hexahydro-3H-inden-4-one (220 mg, 0.61 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (900 mg) after evaporation of the solvent was purified by FC (15 g, 10% AcOEt in hexane) to give 1-alpha,3-beta-Di(tert-Butyl-dimethyl-silanyloxy)-25-trimethylsilanyloxy-16-ene-20-cyclopropyl-19-nor-cholecalciferol (421 mg, 0.59 mmol). To the 1-alpha,3-beta-Di(tert-Butyl-dimethyl-silanyloxy)-25-trimethylsilanyloxy-16-ene-20-cyclopropyl-26,27-hexadeutero-19-nor-cholecalciferol (421 mg, 0.59 mmol) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (450 mg) after evaporation of the solvent was purified by FC (15 g, 50% AcOEt in hexane and AcOEt) to give the titled compound (31) (225 mg, 0.54 mmol, 89%). [α]²⁹_{D}=+ 69.5 c 0.37, EtOH. UV λmax (EtOH): 243 nm (ε 27946), 251 nm (ε33039), 261 nm (ε227R(CDCl₃): 6.30 (1H, d, J=11.3 Hz), 5.93 (1H, d, J=11.3 Hz), 5.36 (1H, m), 4.12, (1H, m), 4.04 (1H, m), 2.75 (2H, m), 2.52-1.04 (22H, m), 1.18 (6H, s), 0.79 (3H, s), 0.65-0.26 (4H, m); ¹³C NMR (CDCl₃): 157.16(0), 142.33 (0), 131.25 (0), 124.73(1), 123.76 (1), 115.50 (1), 71.10 (0), 67.39 (1), 67.19 (1), 59.47 (1), 50.12 (0), 44.60 (2), 43.84 (2), 42.15 (2), 38.12 (2), 37.18 (2), 35.57 (2), 29.26 (3), 29.11 (2), 29.08 (3), 28.48 (2), 23.46 (2), 22.26 (2), 21.27 (0), 17.94 (3), 12.70 (2), 10.27 (2); MS HRES Calculated for C₂₇H₄₂O₃ M+H 415.3207. Observed M+H 415.3207.

### Synthetic Example 40

### Synthesis of 1-alpha,25-Dihydroxy-16-ene-20-cyclopropyl-cholecalciferol (32)

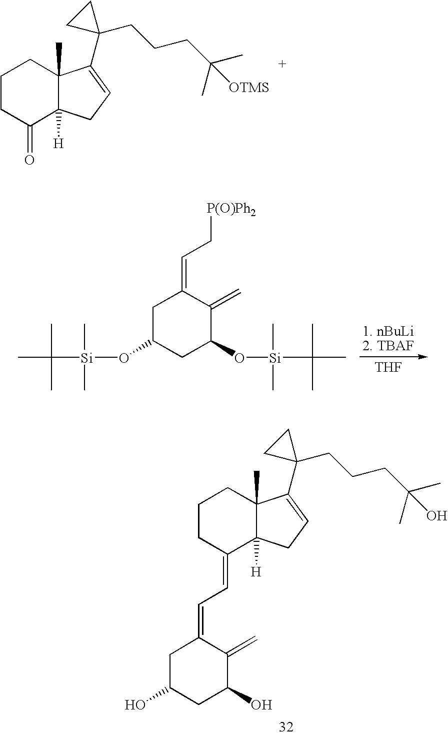

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (675 mg, 1.16 mmol) in tetrahydrofurane (8 mL) at −78° C. was added n-BuLi (0.73 mL, 1.17 mmol). The resulting mixture was stirred for 15 min and solution of (3aR,7aR)-7a-Methyl-1-[1-(4-methyl-4-trimethylsilanyloxy-pentyl)-cyclopropyl]-3a,4,5,6,7,7a-hexahydro-3H-inden-4-one (210 mg, 0.58 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (850 mg) after evaporation of the solvent was purified by FC (15 g, 10% AcOEt in hexane) to give 1-alpha,3-beta-Di(tert-Butyl-dimethyl-silanyloxy)-25-trimethylsilanyloxy-16-ene-20-cyclopropyl-cholecalciferol (382 mg, 0.53 mmol). To the 1-alpha,3-beta-Di(tert-Butyl-dimethyl-silanyloxy)-25-trimethylsilanyloxy-16-ene-20-cyclopropyl-cholecalciferol (382 mg, 0.53 mmol) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (380 mg) after evaporation of the solvent was purified by FC (15 g, 50% AcOEt in hexane and AcOEt) to give the titled compound (32) (204 mg, 0.48 mmol, 83%). [α]²⁹_{D}=+ 16.1 c 0.36, EtOH. UV λmax (EtOH): 208 nm (ε 17024), 264 nm (ε 16028); ¹H NMR (CDCl₃): 6.37 (1H, d, J=11.3 Hz), 6.09 (1H, d, J=11.1 Hz), 5.33 (2H, m), 5.01 (1H, s), 4.44 (1H, m), 4.23 (1H, m), 2.80 (1H, m), 2.60 (1H, m), 2.38-1.08 (20H, m), 1.19 (6H, s), 0.79 (3H, s), 0.66-0.24 (4H, m); ¹³C NMR (CDCl₃): 157.07(0), 147.62 (0), 142.49 (0), 133.00 (0), 124.90(1), 124.73(1); 117.19 (1), 111.64 (2), 71.10 (1), 70.70 (0), 66.88 (1), 59.53 (1), 50.28 (0), 45.19 (2), 43.85 (2), 42.86 (2), 38.13 (2), 35.59 (2), 29.27 (2), 29.14 (3), 28.65 (2), 23.57 (2), 22.62 (2), 21.29 (0), 17.84 (3), 12.74 (2), 10.30 (2); MS HRES Calculated for C₂₈H₄₂O₃ M+Na 449.3026. Observed M+Na 449.3023.

### Synthetic Example 41

### Synthesis of 1,25-Dihydroxy-21-(2R,3-dihydroxy-3-methyl-butyl)-20R-Cholecalciferol (33)

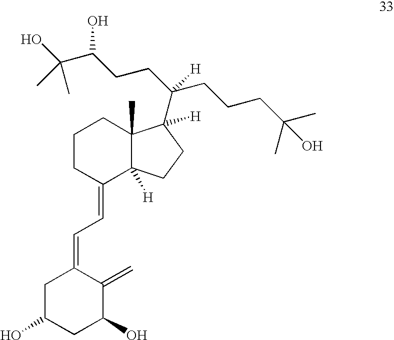

### [1R,3aR,4S,7aR]-2(R)-[4-(1,1-dimethylethyl)dimethyl-silanyloxy)-7a-methyl-octahydro-inden-1-yl]-6-methyl-heptane-1,6-diol (34) and [1R,3aR,4S,7aR]-2(S)-[4-(1,1-dimethylethyl)dimethyl-silanyloxy)-7a-methyl-octahydro-inden-1-yl]-6-methyl-heptane-1,6-diol (35)

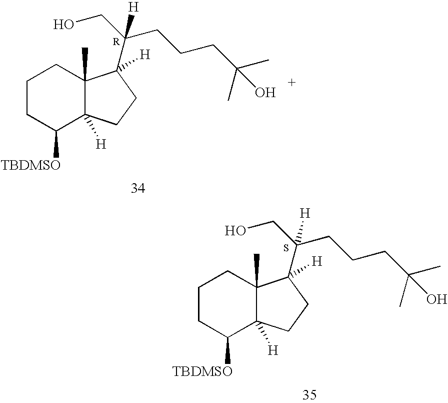

A solution of the alkenol in tetrahydrofuran (9 mL) was cooled in an ice bath and a 1M solution of borane-THF in tetrahydrofuran (17 mL) was added dropwise in an originally effervescent reaction. The solution was stirred overnight at room temperature, re-cooled in an ice bath water (17 mL) was added dropwise followed by sodium percarbonate (7.10 g, 68 mmol). The mixture was immersed into a 50° C. bath and stirred for 70 min to generate a solution. The two-phase system was allowed to cool then equilibrated with 1:1 ethyl acetate-hexane (170 mL). The organic layer was washed with water (2×25 mL) then brine (20 mL), dried and evaporated to leave a colorless oil (2.76 g). This material was passed through a short flash column using 1:1 ethyl acetate-hexane and silica gel G. The effluent, obtained after exhaustive elution, was evaporated, taken up in ethyl acetate, filtered and chromatographed on the 2×18″ 15-20μ silica YMC HPLC column using 2:1 ethyl acetate-hexane as mobile phase and running at 100 mL/min. Isomer 34 emerged at an effluent maximum of 2.9 L, colorless oil, 1.3114 g, [α]_{D}+45.2° (methanol, c 0.58; ¹H NMR δ −0.002 (3H, s), 0.011 (3H, s), 0.89 (9H, s), 0.93 (3H, s), 1.17 (1H, m), 1.22 (6H, s), 1.25-1.6 (16H, m), 1.68 (1H, m), 1.80 (2H, m), 1.89 (1H, m), 3.66 (1H, dd, J=4.8 and 11 Hz), 3.72 (1H, dd, J=3.3 and 11 Hz), 4.00 (1H, m); LR-ES(−) m/z 412 (M), 411 (M−H); HR-ES(+): calcd for (M+Na) 435.3265, found: 435.3269.

Isomer 35 at was eluted at an effluent maximum of 4.9 L, colorless oil, 0.8562 g that crystallized upon prolonged standing: mp 102-30, [α]_{D}+25.20 (methanol, c 0.49); ¹H NMR δ 0.005 (3H, s), 0.009 (3H, s), 0.89 (9H, s), 0.93 (3H, s), 1.16 (1H, m), 1.22 (6H, s), 1.3-1.5, (14H, m), 1.57 (2H, m), 1.67 (1H, m), 1.80 (2H, m), 1.91 (1H, m), 3.54 (1H, dd, J=4.8 and 11 Hz), 3.72 (1H, dd, J=2.9 and 11 Hz), 4.00 (1H, m); LR-ES(−) m/z 412 (M), 411 (M−H). Anal. Calcd for C₂₄H₄₈O₃Si: C, 69.84, H, 11.72; found: C, 69.91; H, 11.76.

### [1R,3aR,4S,7aR]-6(R)-[4-tert-Butyl-dimethyl-silanyloxy)-7a-methyl-octahydro-inden-1-yl]-7-iodo-2-methyl-heptan-2-ol (36)

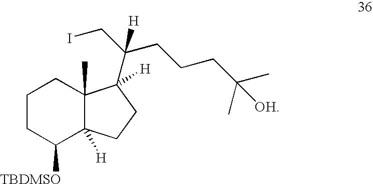

A stirred mixture of triphenylphosphine (0.333 g, 1.27 mmol) and imidazole (0.255 g, 3 mmol) in dichloromethane (3 mL) was cooled in an ice bath and iodine (0.305 g, 1.20 mmol) was added. This mixture was stirred for 10 min then a solution of 34 (0.4537 g, 1.10 mmol) in dichloromethane (3 mL) was added dropwise over a 10 min period. The mixture was stirred in the ice bath for 30 min then at ambient temperature for 2¾ h. TLC (1:1 ethyl acetate-hexane) confirmed absence of educt. A solution of sodium thiosulfate (0.1 g) in water (5 mL) was added, the mixture equilibrated and the organic phase washed with 0.1 N sulfuric acid (10 mL) containing a few drops of brine then with 1:1 water-brine (2×10 mL), once with brine (10 mL) then dried and evaporated. The residue was purified by flash chromatography using 1:9 ethyl acetate-hexane as mobile phase to furnish 36 as a colorless syrup, 0.5637 g, 98%: ¹H NMR δ 0.005 (3H, s), 0.010 (3H, s), 0.89 (9H, s), 0.92 (3H, s), 1.23 (6H, s), 1.1-1.6 (16H, m), 1.68 (1H, m), 1.79 (2H, m), 1.84 (1H, m), 3.37 (1H, dd, J=4 and 10 Hz), 3.47 (1H, dd, J=3 and 10 Hz), 4.00 (1H, m); LR-EI(+) m/z 522 (M), 465 (M-C₄H₉), 477 (M-C₄H₉—H₂O); HR-EI(+): calcd for C₂₄H₄₇IO₂Si: 522.2390, found: 522.2394.

### [1R,3aR,4S,7aR]-6(S)-[4-(tert-Butyl-dimethyl-silanyloxy)-7a-methyl-octahydro-inden-1-yl]-2-methyl-non-8-yn-2-ol (37)

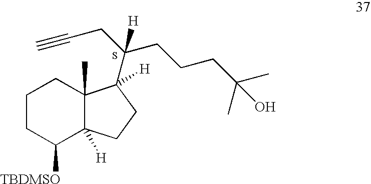

Lithium acetylide DMA complex (0.110 g, 1.19 mmol) was added to a solution of 36 (0.2018 g (0.386 mmol) in dimethyl sulfoxide (1.5 mL) and tetrahydrofuran (0.15 mL). The mixture was stirred overnight. TLC (1:4 ethyl acetate-hexane) showed a mixture of two spots traveling very close together (Rf 0.52 and 0.46). Fractions at the beginning of the eluted band contained pure alkenol, which is the elimination product of 36, and was produced as the major product. Fractions at the end of the elution band, however, were also homogeneous and gave the desired acetylene 37 upon evaporation. The NMR spectra of 37 and its 6-epimer which served for identification were previously reported.

### [1R,3aR,4S,7aR]-7-Benzenesulfonyl-6(S)-[4-(tert-butyl-dimethyl-silanyloxy)-7a-methyl-octahydro-inden-1-yl]-2-methyl-heptan-2-ol (38)

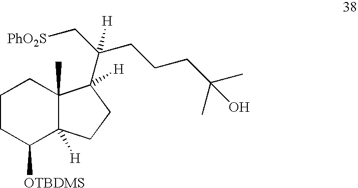

A mixture of 37b (0.94 g, 1.8 mmol), sodium benzenesulfinate (2.18 g, 13 mmol) and N,N-dimethylformamide (31.8 g) was stirred at room temperature for 12 h, then in a 40° C. bath for ca.6 h until all educt was converted as shown by TLC (1:4 ethyl acetate-hexane). The solution was equilibrated with 1:1 ethyl acetate-hexane (120 mL) and 1:1 brine-water (45 mL). The organic layer was washed with water (4×25 mL) brine (10 mL), then dried and evaporated to leave a colorless oil, 1.0317 g. This material was flash-chromatographed using a stepwise gradient (1:9, 1:6, 1:3 ethyl acetate-hexane) to give a colorless oil, 0.930 g, 96%: 300 MHz ¹H NMR δ −0.02 (3H, s), 0.00 (3H, s), 0.87 (9H, s), 0.88 (3H, s), 1.12 (1H, m), 1.20 (6H, s), 1.2-1.8 (18H, m), 1.81 (1H, m), 3.09 (2H, m), 3.97 (1H, brs), 7.59 (3H, m), 7.91 2H, m).

### [1R,3aR,4S,7aR]-1-(1(S)-Benzenesulfonylmethyl-5-methyl-5-trimethylsilanyloxy-hexyl)-4-(tert-butyl-dimethyl-silanyloxy)-7a-methyl-octahydro-indene (39)

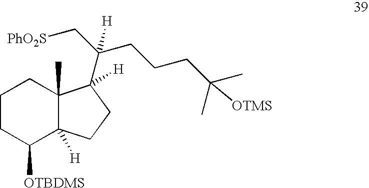

1-(Trimethylsilyl)imidazole (1 mL) was added to a solution of 38 (0.8 g) in cyclohexane (10 mL) and stirred overnight then flash-chromatographed using a stepwise gradient of hexane, 1:39 and 1:19 ethyl acetate-hexane. The elution was monitored by TLC (1:4 ethyl acetate-hexane) leading to 39 as a colorless syrup, 0.7915 g: 300 MHz ¹H NMR δ 0.00 (3H, s), 0.02 (3H, s), 0.12 (9H, s), 0.90 (12H, s, t-butyl+7a-Me), 1.16 (1H, m), 1.20 (6H, s), 1.2-1.6 (15H, m), 1.66-1.86 (3H, m), 3.10 (2H, m), 4.00 (1H, brs), 7.56-7.70 (3H, m), 7.93 (2H, m).

### [1R,3aR,4S,7aR]-6(R)-[4-tert-Butyl-dimethyl-silanyloxy)-7a-methyl-octahydro-inden-1-yl]-2,10 dimethyl-undecane-2,3(R), 10-triol (40)

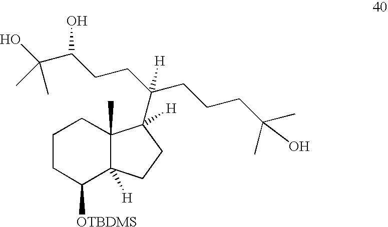

A solution of 39 (0.7513 g, 1.23 mmol) and diol (0.508 g, 1.85 mmol) in tetrahydrofuran (28 mL) was cooled to −35° C. then 2.5 M butyllithium in hexane (2.75 mL) was added dropwise. The temperature was allowed to rise to −20° C. and maintained at that temperature for 6 h or until the educt was consumed. Reaction progress was monitored by TLC (1:4 ethyl acetate-hexane) exhibiting the educt (Rf 0.71) and the two epimeric diols (Rf 0.09 and 0.12). Toward the end of the reaction period the temperature was increased briefly to 0° C., lowered again to −10, then saturated ammonium chloride (25 mL) was added followed by ethyl acetate (50 mL) and enough water to dissolve the precipitated salts. The resulting aqueous phase was extracted with ethyl acetate (15 mL). The combined extracts were washed with brine (15 mL), dried and evaporated. The resulting syrup was flash-chromatographed using a stepwise gradient of 1:9, 1:6, 1:4 and 1:1 ethyl acetate-hexane to give 39a as a colorless syrup, 0.8586 g. This material was dissolved in a mixture of tetrahydrofuran (30 mL) and methanol (18 mL), then 5% sodium amalgam (20 g) was added. The reductive de-sulfonylation was complete after stirring of the mixture for 14 h. Progress of the reaction was monitored by TLC (1:1 ethyl acetate-hexane) which showed the disappearance of the epimeric diols (Rf 0.63 and 0.74) and the generation of 40a (Rf 0.79) and the partially de-silylated analog 40 (Rf 0.16). The mixture was diluted with methanol (20 mL), stirred for 3 min, then ice (20 g) was added, stirred for 2 min and the supernatant decanted into a mixture containing saturated ammonium chloride (50 mL). The residue was repeatedly washed with small amounts of tetrahydrofuran that was also added to the salt solution, which was then equilibrated with ethyl acetate (80 mL). The aqueous layer was re-extracted once with ethyl acetate (20 mL), the combined extracts were washed with brine (10 mL) then dried and evaporated. The resulting colorless oil containing both 40a and 40 was dissolved in 10 mL of a 1 N oxalic acid solution in methanol (prepared from the dihydrate) effecting the selective hydrolysis of the trimethylsilyl ether within minutes. Calcium carbonate (1 g) was added and the suspension stirred overnight, then filtered. The solution was evaporated and the resulting residue flash-chromatographed using a stepwise gradient of 1:4, 1:2, 1:1 and 2:1 ethyl acetate-hexane giving a residue of the triol 40 that crystallized in very fine branching needles from acetonitrile, 0.45 g: mp 94-95° C., [α]_{D}+44.1° (methanol, c 0.37); 400 MHz ¹H NMR δ −0.005 (3H, s), 0.007 (3H, s), 0.89 (9H, s), 0.92 (3H, s), 1.15 (1H, m), 1.16 (3H, s), 1.21 (9H, s), 1.2-1.6 (19H, m), 1.67 (1H, m), 1.79 (2H, m), 1.90 (2H, m), 2.06 (1H, m), 3.31 (1H, brd, J=10 Hz), 4.00 (1H, brs), LR-ES(−) m/z: 533 (M+Cl), 497 (M−H); HR-ES(+): Calcd for C₂₉H₅₈O₄Si+Na: 521.3996, found: 521.4003. Anal Calcd for C₂₉H₅₈O₄Si: C, 69.82, H, 11.72; found: C, 69.97; H, 11.65.

[1R,3aR,4S,7aR]-6(R)-(4-Hydroxy-7a-methyl-octahydro-inden-1-yl)-2,10-dimethyl-undecane-2,3(R), 10-triol (41).

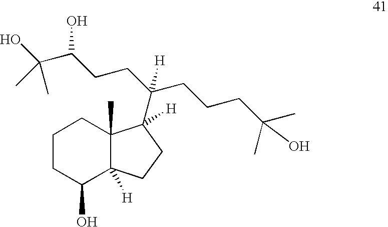

A stirred solution of the triol 40 (0.4626 g, 0.927 mmol) in acetonitrile (10 mL) and dioxane (0.7 mL) was cooled to 10° C. and a fluorosilicic acid solution (2 mL) was added dropwise. The cooling bath was removed, the 2-phase system further diluted with acetonitrile (2 mL) then stirred at room temperature for 3¼ h. The disappearance of educt was monitored by TLC (ethyl acetate). The mixture was equilibrated with water (10 mL) and ethyl acetate (30 mL). The aqueous phase was re-extracted with ethyl acetate (2×20 mL), the combined extracts were washed with water (5 mL) and brine (10 mL), then 1:1 brine-saturated sodium hydrogen carbonate solution and dried. The residue was purified by flash-chromatography using a step-wise gradient from 1:1 to 2:1 ethyl acetate-hexane and neat ethyl acetate to give a residue that was taken up in 1:1 dichloromethane-hexane, filtered and evaporated to furnish amorphous solids, 0.3039 g (85%): [α]_{D}+42.60 (methanol, c 0.48); ¹H NMR (DMSO-d₆): δ 0.87 (3H, s), 0.97 (3H, s), 1.02 (3H, s), 1.04 (6H, s), 1.1-1.4 (18H, m), 1.5-1.8 (4H, m), 1.84 (1H, m), 2.99 (1H, dd, J=6 and 10 Hz), 3.87 (1H, brs), 4.02 (1H, s, OH), 4.05 (1H, s, OH), 4.16 (1H, d, OH, J=3.6 Hz), 4.20 (1H, d, OH, J=6.4 Hz); LR-ES(+): m/z 384 (M), 383 (M−H); HR-ES(+): Calcd for (M+Na) 407.3132, found: 407.3134.

### [1R,3aR,4S,7aR]-1-{5-Hydroxy-5-methyl-1(R)-[2-(2,2,5,5-tetramethyl-[1,3]-dioxolan-4(R)-yl)-ethyl]-hexyl}-7a-methyl-octahydro-inden-4-ol (42)

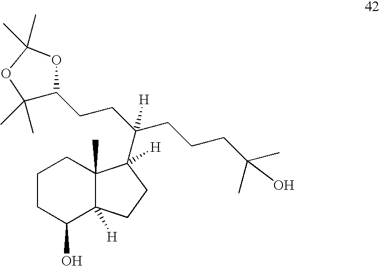

A solution of the tetraol 40 (0.2966 g, 0.771 mmol) and pyridinium tosylate (100 mg) in acetone (8 mL) and 2,2-dimethoxypropane (8 mL) was kept at room temperature for 12 h. TLC analysis (ethyl acetate) showed the absence of educt (Rf 0.21) and two new spots with Rf 0.82 and 0.71, the former the expected 42 and the latter assumed to be the methylacetal. The reaction mixture was diluted with water (5 mL) and stirred for 10 min. At that time only the spot with higher Rf value was observed. The mixture was neutralized with sodium hydrogen carbonate (0.5 g) then equilibrated with ethyl acetate (50 mL) and brine (5 mL). The organic layer was washed with water (5 mL) and brine (5 mL) then dried and evaporated to leave a sticky residue (0.324 g) that was used directly in the next step: 300 MHz ¹H NMR: δ 0.94 (3H, s), 1.10 (3H, s), 1.20 (1H, m), 1.22 (6H, s), 1.25 (3H, s), 1.34 (3H, s), 1.41 (3H, s), 1.2-1.65 (20H, m), 1.78-1.86 (3H, m), 1.93 (1H, m), 3.62 (1H, dd, J=4.6 and 8.3 Hz), 4.08 (1H, brs).

### [1R,3aR,4S,7aR]-Acetic acid 1-{5-hydroxy-5-methyl-1(R)-[2-(2,2,5,5-tetramethyl-[1,3]dioxolan-4(R)-yl)-ethyl]-hexyl}-7a-methyl-octahydro-inden-4-yl ester (43)

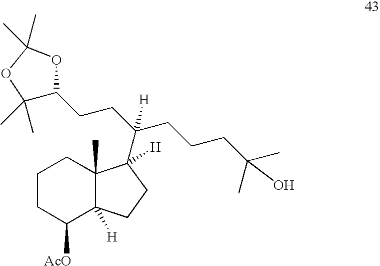

The residue obtained above was dissolved in pyridine (6.9 g) and further diluted with acetic anhydride (3.41 g). The mixture was allowed to stand at room temperature for 24 h, then in a 35° C. bath for ca. 10 h until the educt was no longer detectable (TLC, ethyl acetate). The mixture was diluted with toluene and evaporated. The residue was purified by flash chromatography (1:4 ethyl acetate-hexane) to give 43 as colorless syrup, 0.3452 g, 97%: ¹H NMR: δ 0.89 (3H, s), 1.10 (3H, s), 1.20 (1H, m), 1.22 (6H, s), 1.25 (3H, s), 1.33 (3H, s), 1.41 (3H, s), 1.25-1.6 (19H, m), 1.72 (1H, m), 1.82 (2H, m), 1.95 (1H, m), 2.05 (3H, s), 3.63 (1H, dd, J=4.4 and 8.4 Hz), 5.15 (1H, brs); LR-FAB(+) m/z 467 (M+H), 465 (M−H), 451 (M-Me).

### [1R,3aR,4S,7aR]-Acetic acid 1-[4(R), 5-dihydroxy-1(R)-(4-hydroxy-4-methyl-pentyl)-5-methyl-hexyl]-7a-methyl-octahydro-inden-4-yl ester (44)

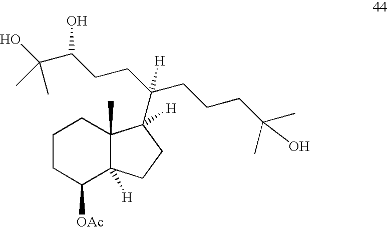

A solution of 43 (0.334 g, 0.716 mmol) in 80% acetic acid (2 mL) was kept in a 68° C. bath. TLC (ethyl acetate, Rf 0.33) monitored the progress of the hydrolysis. The educt was no longer detectable after 2.5 h. The mixture was evaporated then co-evaporated with a small amount of toluene to leave a colorless film (0.303 g) that was used directly in the next step: 300 MHz ¹H NMR: δ 0.89 (3H, s), 1.17 (3H, s), 1.22 (6H, s), 1.56 (3H, s), 1.1-1.6 (21H, m), 1.6-2.0 (5H, m), 2.04 (3H, s), 3.32 (1H, brd, J=10 Hz), 5.15 (1H, brs).

### [1R,3aR,4S,7aR]-Acetic acid 1-[4(R)-[dimethyl-(1,1,2-trimethyl-propyl)-silanyloxy]-5-hydroxy-1(R)-(4-hydroxy-4-methyl-pentyl)-5-methyl-hexyl]-7a-methyl-octahydro-inden-4-yl ester (45)

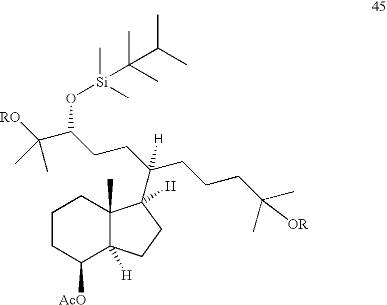

A solution of the triol 44 (0.30 g), imidazole (0.68 g, 10 mmol) and dimethylthexylsilyl chloride (1.34 g, 7.5 mmol) in N,N-dimethylformamide (6 g) was kept at room temperature. After 48 h 4-(N,N-dimethylamino)pyridine (15 mg) was added and the mixture stirred for an additional 24 h. Reaction progress was monitored by TLC (ethyl acetate; 24, Rf 0.83; 25a, Rf 0.38). The mixture was diluted with water (2 mL), stirred for 10 min then distributed between ethyl acetate (45 mL) and water (20 mL). The aqueous layer was extracted once with ethyl acetate (10 mL). The combined organic phases were washed with water (4×12 mL) and brine (8 mL) then dried and evaporated. The residual oil was purified by flash-chromatography using a stepwise gradient of 1:9 and 1:4 ethyl acetate-hexane to give 45 as colorless syrup. A small amount of unreacted educt (80 mg) was eluted with ethyl acetate. The syrupy 45 was used directly in the next step: 400 MHz ¹H NMR: δ 0.13 (3H, s), 0.14 (3H, s), 0.87 (6H, s), 0.91 (9H, m), 1.10 (1H, m), 1.14 (3H, s), 1.15 (3H, s), 1.21 (6H, s), 1.1-1.6 (19H, m), 1.6-1.9 (5H, m), 1.94 (1H, brd, J=12.8 Hz), 2.05 (3H, s), 3.38 (1H, brs), 5.15 (1H, brs).

### [1R,3aR,4S,7aR]-Acetic acid 1-[4(R)-[dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-yl ester (46)

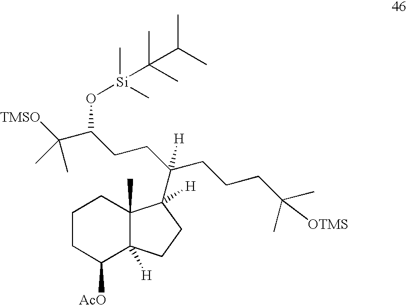

1-(Trimethylsilyl)imidazole (0.90 mL, 6.1 mmol) was added to a solution of 45 (0.2929 mg) in cyclohexane (6 mL) and stirred for 12 h, then flash-chromatographed (1:79 ethyl acetate-hexane) to yield 46 as colorless syrup (0.3372 g). The elution was monitored by TLC (1:4 ethyl acetate-hexane) leading to 46 as a colorless syrup, 0.7915 g: ¹H NMR δ: 0.074 (3H, s), 0.096 (3H, s), 0.103 (9H, s), 0.106 (9H, s), 0.82 (1H, m), 0.83 (6H, s), 0.88 (9H, m), 1.32 (3H, s), 1.20 (9H, s), 1.15-1.6 (17H, m), 1.6-1.9 (5H, m), 1.97 (1H, brd, J=12.8 Hz), 2.05 (3H, s), 3.27 (1H, m), 5.15 (1H, brs); LR-FAB(+) m/z: 712 (M), 711 (M−H), 697 (M-Me), 653 (M-AcO), 627 (M-C₆H₁₃).

### [1R,3aR,4S,7aR]-1-[4(R)-[Dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-ol (47)

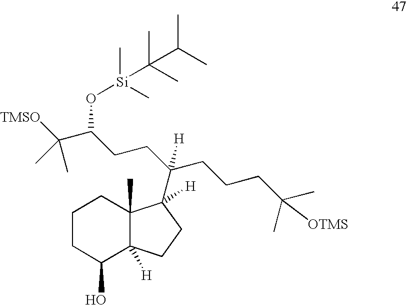

A stirred solution of 46 (0.335 mg, 0.47 mmol) in tetrahydrofuran (15 mL) was cooled in an ice-bath and a 1 M solution of lithium aluminum hydride in tetrahydrofuran (2 mL) was added dropwise. TLC (1:9 ethyl acetate-hexane) showed complete conversion 25b (Rf 0.61) to 26 (Rf 0.29) after 1.5 h. A 2 M sodium hydroxide solution (14 drops) was added, followed by water (0.5 mL) and ethyl acetate (30 mL). A small amount of Celite was added and, after stirring for 15 min, the liquid layer was filtered off. The solid residue was rinsed repeatedly with ethyl acetate and the combined liquid phases evaporated to leave a colorless syrup, that was taken up in hexane, filtered and evaporated to yield 26 (0.335 g) that was used without further purification: ¹H NMR δ: 0.075 (3H, s), 0.10 (21H, brs), 0.82 (1H, m), 0.84 (6H, s), 0.89 (6H, m), 0.93 (3H, s), 1.13 (3H, s), 1.20 (9H, s), 1.2-1.6 (16H, m), 1.6-1.7 (2H, m), 1.82 (3H, m), 1.95 (1H, brd, J=12.4 Hz), 3.27 (1H, m), 4.08 (1H, brs); LR-FAB(+) m/z: 585 (M-C₆H₁₃), 481 (M-TMSO); HR-ES(+) m/z: Calcd for C₃₇H₇₈O₄Si₃+Na: 693.5100 found: 693.5100.

### [1R,3aR,7a]-1-[4(R)-[Dimethyl-(1,1,2-trimethyl-propyl)-silanyloxy]-5-methyl-1 (R)-(4-methyl-4-trimethylsilanyloxy-pentyl)-5-trimethylsilanyloxy-hexyl]-7a-methyl-octahydro-inden-4-one (48)

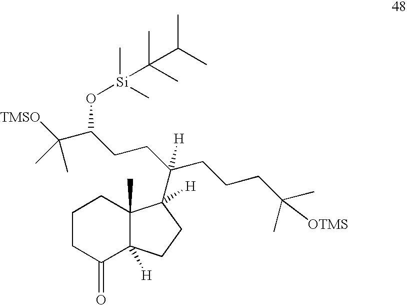

Celite (0.6 g) was added to a stirred solution of 47 (0.310 g, 0.462 mmol) in dichloromethane (14 mL) followed by pyridinium dichromate (0.700 g, 1.86 mmol). The conversion of 47 (Rf 0.54) to the ketone 27 (Rf 0.76) was followed by TLC (1:4 ethyl acetate-hexane). The mixture was diluted with cyclohexane after 4.5 h then filtered trough a layer of silica gel. Filtrate and ether washes were combined and evaporated. The residue was flash-chromatographed (1:39 ethyl acetate-hexane) to give 27 as a colorless syrup, 0.2988 g, 96.6%: ¹H NMR δ: 0.078 (3H, s), 0.097 (3H, s), 0.107 (18H, s), 0.64 (3H, s), 0.81 (1H, m), 0.84 (6H, s), 0.89 (6H, m), 1.134 (3H, s), 1.201 (3H, s), 1.207 (3H, s), 1.211 (3H, s), 1.3-1.6 (14H, m), 1.6-1.7 (3H, m), 1.88 (1H, m), 2.04 (2H, m), 2.2-2.32 (2H, m), 2.46 (1H, dd, J=7.5 and 11.5 Hz), 3.28 (1H, m); LR-FAB(+) m/z: 583 (M-C₆H₁₃), 479 (M-OTMS); HR-ES(+) m/z: Calcd for C₃₇H₇₆O₄Si₃+Na: 691.4943, found: 691.4949.

### [1R,3aR,7aR,4E]-4-{2(Z)-[3(S),5(R)-Bis-(tert-butyl-dimethyl-silanyloxy)-2-methylene-cyclohexylidene]-ethylidene}-7a-methyl-1-[5-methyl-1(R)-(4-methyl-4-trimethylsilanyloxy-pentyl)-4(R)-[dimethyl-(1,1,2-trimethyl-propyl)-silanyloxy]-5-trimethylsilanyloxy-hexyl]-octahydro-indene (49)

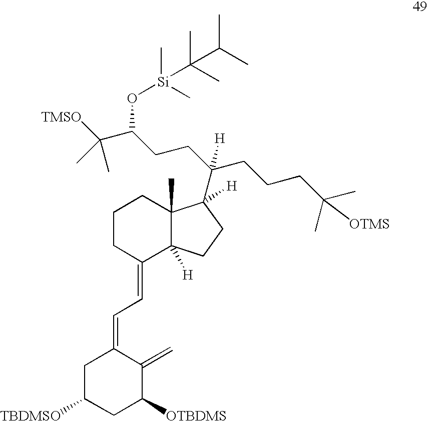

A solution of 2.5-M butyllithium in hexane (0.17 mL) was added to a solution of 28 in tetrahydrofuran (2 mL) at −70° C. to produce a deep cherry-red color of the yield. After 10 min a solution of ketone 27 (0.1415 g, 0.211 mmol) in tetrahydrofuran (2 mL) was added dropwise over a 15 min period. The reaction was quenched after 4 h by the addition of pH 7 phosphate buffer (2 mL). The temperature was allowed to increase to 0° C. then hexane (30 mL) was added. The aqueous layer was re-extracted with hexane (15 mL). The combined extracts were washed with of brine (5 mL), dried and evaporated to give a colorless oil that was purified by flash-chromatography (1:100 ethyl acetate-hexane) to yield 49 as colorless syrup, 0.155 g, 71%: ¹H NMR δ: 0.068 (15H, m), 0.103 (12H, s), 0.107 (9H, s), 0.53 (3H, s), 0.82 (1H, m), 0.84 (6H, s), 0.88 (18H, m), 0.89 (6H, m), 1.14 (3H, m), 1.20 (9H, s), 12-1.9 (22H, m), 1.97 (2H, m), 2.22 (1H, dd, J=7.5 an 13 Hz), 2.45 (1H, brd, J=13 Hz), 2.83 (1H, brd, J=13 Hz), 3.28 (1H, m), 4.20 (1H, m), 4.38 (1H, m), 4.87 (1H, d, J=2 Hz), 5.18 (1H, d, J=2 Hz), 6.02 (1H, d, J=11.4 Hz, 6.24 (1H, d, J=11.4 Hz); LR-FAB(+) m/z 1033 (M+H), 1032 (M), 1031 (M−H), 901 (M-TBDMS).

### Synthesis of 1,25-Dihydroxy-21-(2R,3-dihydroxy-3-methyl-butyl)-20R-Cholecalciferol (33)

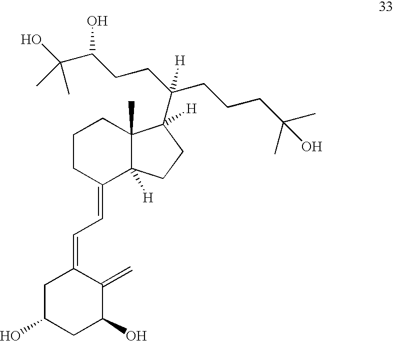

The residue of 49 (0.153 g, 0.148 mmol), as obtained in the previous experiment, was dissolved in a 1 M solution of tetrabutylammonium fluoride (3.5 mL). TLC (ethyl acetate) monitored reaction progress. Thus, the solution was diluted with brine (5 mL) after 24 h, stirred for 5 min then equilibrated with ethyl acetate (35 mL) and water (15 mL). The aqueous layer was re-extracted once with ethyl acetate (15 mL). The combined organic layers were washed with water (5×10 mL), once with brine (5 mL) then dried and evaporated. The residue was purified by flash chromatography using a stepwise gradient of ethyl acetate and 1:100 methanol-ethyl acetate furnishing 33 as colorless, microcrystalline material from methyl formate-pentane, 70 mg, 91%: [α]_{D}+34.3° (methanol, c 0.51); ¹H NMR (DMSO-d₆) δ: 0.051 (3H, s), 0.98 (3H, s), 1.03 (3H, s), 1.05 (6H, s), 1.0-1.6 (17H, m), 1.64 (3H, m), 1.80 (2H, m), 1.90 (1H, d, J=11.7 Hz), 1.97 (1H, dd, J=J=9.8 Hz), 2.16 (1H, dd, J=5.9 and J=13.7 Hz), 2.36 (1H, brd), 2.79 (1H, brd), 3.00 (1H, dd, J=5 and 10 Hz), 3.99 (1H, brs), 4.01 (1H, s, OH), 4.04 (1H, s, OH), 4.54 (1H, OH, d, J=3.9 Hz), 4.76 (1H, brs), 4.87 (1H, OH, d, J=4.9 Hz), 5.22 (1H, brs), 5.99 (1H, d, J=10.7 Hz), 6.19 (1H, d, J=10.7 Hz); LR-ES(+) m/z: 519 (M+H), 518 (M), 517 (M−H), 501 (M-OH); HR-ES(+) calcd for C₃₂H₅₄O₅+Na: 541.3863; found 541.3870; UVₘₐₓ(ε): 213 (13554), 241sh (12801), 265 (16029) nm.

### Synthetic Example 42

### Synthesis of 1,25-Dihydroxy-21(2R,3-dihydroxy-3-methyl-butyl)-20S-Cholecalciferol (50)

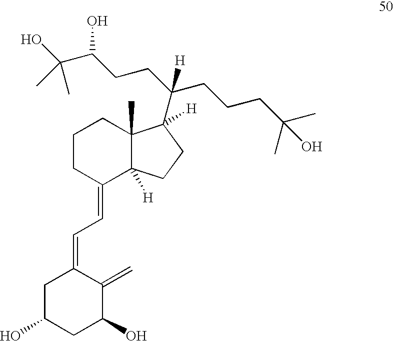

### [1R,3aR,4S,7aR]-7-Benzenesulfonyl-6(R)-[4-(tert-butyl-dimethyl-silanyloxy)-7a-methyl-octahydro-inden-1-yl]-2-methyl-heptan-2-ol (51)

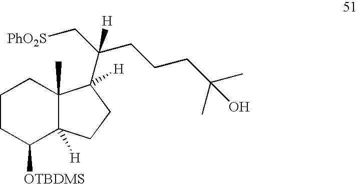

A solution of 36 and sodium benzenesulfinate (0.263 g, 1.6 mmol) in N,N-dimethyl formamide (5 mL) was stirred in a 77° C. bath for 3 h. The solution was equilibrated with 1:1 ethyl acetate-hexane (25 mL) and the organic layer washed with water (5×10 mL), dried and evaporated. The residue was flash-chromatographed with a stepwise gradient of 1:9, 1:4, and 1:3 ethyl acetate-hexane to furnish the sulfone as a colorless syrup: ¹H NMR δ −0.02 (3H, s), 0.005 (3H, s), 0.79 (3H, s), 0.87 (9H, s), 1.12 (1H, m), 1.19 (6H, s), 1.12 (1H, m), 1.20 (6H, s), 1.2-1.8 (18H, m), 2.08 (1H, m), 3.09 (1H, dd, J=9.3 and 14.5 Hz), 3.31 (1H, dd, J=3 and 14.5 Hz), 3.97 (1H, brs), 7.58 (3H, m), 7.66 (1H, m), 7.91 2H, m); LR-ES(+) m/z: 600 (M+Na+MeCN), 559 (M+Na); LR-ES(−) m/z: 536 (M), 535 (M−H); HR-ES(+): Calcd for C₃₀H₅₂O₄SSi+Na 559.3248; found 559.3253.

### [1R,3aR,4S,7aR]-1-(1(R)-Benzenesulfonylmethyl-5-methyl-5-trimethylsilanyloxy-hexyl)-4-(tert-butyl-dimethyl-silanyloxy)-7a-methyl-octahydro-indene (52)

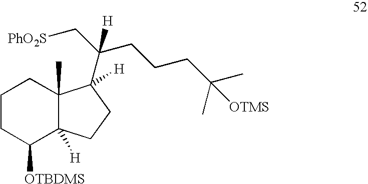

1-(Trimethylsilyl)imidazole (0.146 mL) was added to a solution of 51 (0.145 g, 0.27 mmol) in cyclohexane (2 mL). After 17 h the product was purified by flash chromatography using a stepwise gradient of 1:79 and 1:39 ethyl acetate-hexane to give 52 as colorless residue, 0.157 g 0.258 mmol, TLC (1:9 ethyl acetate-hexane) Rf 0.14. 300 MHz ¹H NMR: δ −0.02 (3H, s), 0.00 (3H, s), 0.87 (12H, s), 1.12 (1H, m), 1.17 (6H, s), 1.2-1.6 (15H, m), 1.6-1.9 (3H, m), 3.08 (2H, m), 3.97 (1H, brs), 7.53-7.70 (3H, m), 7.90 (2H, d, J=7 Hz).

### [1R,3aR,4S,7aR]-5(R,S)-Benzenesulfonyl-6(R)-[4-(tert-butyl-dimethyl-silanyloxy)-7a methyl-octahydro-inden-1-yl]-2,10-dimethyl-10-trimethylsilanyloxy-undecane-2,3(R)-diol (53)

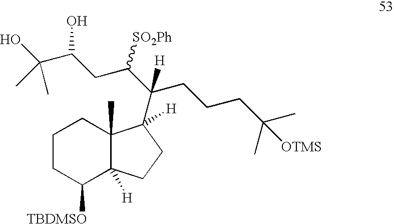

A solution of 52 (0.2589, 0.425 mmol) and diol (0.176 g, 0.638 mmol) in tetrahydrofuran (9 mL) was cooled to −25° C. and 1.6 M butyllithium in hexane (1.4 mL) was added. The temperature was raised to −20° C. and maintained for 3 h then at −10° C. for 2.5 h and 0° C. for 10 min. The mixture was cooled again to −10° C., saturated ammonium chloride solution (5 mL) was added, then equilibrated with ethyl acetate (50 mL) and enough water to dissolve precipitated salts. The aqueous layer was re-extracted with ethyl acetate (15 mL), the combined extracts were dried and evaporated and the residue purified by flash chromatography using a stepwise gradient of 1:6, 1:4, and 1:1 ethyl acetate-hexane to produce 53 as a colorless syrup, 0.212 g, 70%: 300 MHz ¹H NMR: δ 0.00 (3H, s), 0.017 (3H, s), 0.12 (9H, s), 0.81 (3H, s), 0.89 (9H, s), 1.16 (1H, m), 1.19 (12H, m), 1.1-1.6 (20H, m), 1.6-1.8 (2H, m), 3.10 (1H, dd, J=8.4 and 14.7 Hz), 3.30 (1H, m), 3.99 (1H, brs), 7.61 (2H, m), 7.67 (1H, m), 7.93 (2H, m).

### [1R,3aR,4S,7aR]-6(S)-[4-(tert-Butyl-dimethyl-silanyloxy)-7a-methyl-octahydro-inden-1-yl]-2,10-dimethyl-10-trimethylsilanyloxy-undecane-2,3(R)-diol (54)

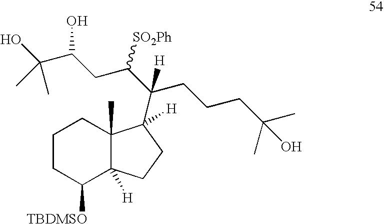

Compound 53 (0.186 mg, 0.262 mmol) was dissolved in 0.5 M oxalic acid dihydrate in methanol (2.5 mL). The solution was stirred for 15 min then calcium carbonate was added (0.5 g) and the suspension stirred overnight then filtered. The filtrate was evaporated to give 54 as a white foam, 0.188 g, 98%: TLC (1:1 ethyl acetate-hexane) Rf 0.06. This material was used in the next step without further purification.

### [1R,3aR,4S,7aR]-6(S)-[4-(tert-Butyl-dimethyl-silanyloxy)-7a-methyl-octahydro-inden-1-yl]-2,10-dimethyl-undecane-2,3(R), 10-triol (triol 55)

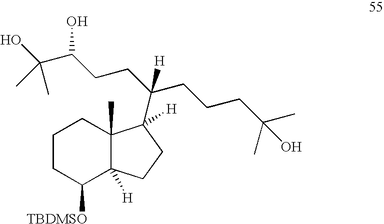

Sodium amalgam (5% sodium, 10.8 g) was added to a vigorously stirred solution of 54 (0.426 g, 0.667 mmol) in a mixture of tetrahydrofuran (15 mL) and methanol (9 mL). The suspension was stirred for 24 h and the reaction monitored by TLC (1:1 ethyl acetate-hexane0 to observe the production of 55 (Rf 0.17). The mixture was diluted with methanol (3 mL), stirred for 5 min then further diluted with water (10 mL), stirred for 2 min and decanted into saturated ammonium chloride solution (25 mL). The aqueous layer was extracted with ethyl acetate (2×20 mL). The combined extracts were washed with pH 7 phosphate buffer (5 mL) then brine (10 mL), dried and evaporated. The residue was purified by flash-chromatography using a stepwise gradient of 1:1 and 2:1 ethyl acetate-hexane to provide 55 as a colorless syrup, 0.244 g, 73%: ¹H NMR: δ −0.006 (3H, s), 0.006 (3H, s), 0.86 (9H, s), 0.92 (3H, s), 1.11 (1H, m), 1.15 (3H, s), 1.21 (9H, s), 1.2-1.75 (21H, m), 1.7-1.85 (3H, m), 1.90 (1H, m), 3.29 (1H, brd), 3.99 (1H, brs); LR-ES(+) m/z: 521 (M+Na), 481 (M-OH); LR-ES(−): m/z 544: (M+CH₂O₂), 543 (M−H+CH₂O₂), 533 (M-Cl); HR-ES(+) m/z: Calcd for CH₅₈O₄Si+Na: 521.3996, found 521.3999.

### [1R,3aR,4S,7aR]-6(S)-(4-Hydroxy-7a-methyl-octahydro-inden-1-yl)-2,10-dimethyl-undecane-2,3(R), 10-triol (56)

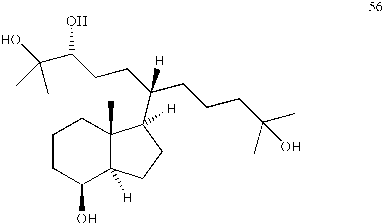

An aqueous fluorosilicic acid solution (3 mL) was added to a stirred solution of 55 (0.240 g, 0.481 mmol) in acetonitrile (12 mL). TLC (ethyl acetate) monitored the reaction. After 2.5 h compound 56 (Rf 0.37) was the predominating species, produced at the expense of less polar 55. The mixture was equilibrated with ethyl acetate and water (10 mL), the aqueous layer was re-extracted with water (2×10 mL) and the combined extracts were washed with water (6 mL) and brine (2×10 mL) then dried and evaporated. The colorless residue was flash-chromatographed using a stepwise gradient of 1:2, 1:1 and 2:1 ethyl acetate-hexane to elute some unreacted 55, followed by 56, obtained as colorless syrup, 0.147 g, 79%: ¹H NMR: 0.94 (3H, s), 1.12 (1H, m), 1.15 (3H, s), 1.21 (9H, s), 1.15-1.7 (20H, m), 1.7-1.9 (5H, m), 1.96 (1H, brd), 3.29 (1H, d, J=9.6 Hz), 4.08 (1H, brs); LR-ES(+): m/z 448: (M+Na+MeCN), 407 (M+Na); LR-ES(−): m/z 419 (M+Cl); HR-ES(+) m/z: Calcd for C₂₃H₄₄O₄+Na: 407.3132, found 407.3135.

### [1R,3aR,4S,7aR]-1-(5-Hydroxy-1(S)-{2-[2-(4-methoxy-phenyl)-5,5-dimethyl-[1,3]dioxolan-4(R)-yl]-ethyl}-5-methyl-hexyl)-7a-methyl-octahydro-inden-4-ol (57)

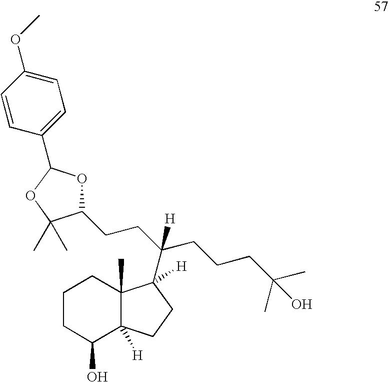

4-Methoxybenzaldehyde dimethyl acetal (60 μL, 0.35 mmol) was added to a solution of 56 (81.2 mg, 0.211 mmol) in dichloromethane (2 mL), followed by a solution (0.2 mL) containing pyridinium tosylate (200 mg) in dichloromethane (10 mL). Reaction progress was followed by TLC (1:2 ethyl acetate-hexane) which showed 4-methoxybenzaldehyde dimethyl acetal (Rf 0.80), 4-methoxybenzaldehyde (Rf 0.65), educt 56 (Rf 0.42) and product 57 (Rf 0.26). After 5¾ h the mixture was stirred for 15 min with saturated sodium hydrogencarbonate solution (5 mL) then equilibrated with ethyl acetate (25 mL). The organic layer was washed with brine (5 mL), dried and evaporated. The residue was flash-chromatographed using a stepwise gradient of 1:3 and 1:2 ethyl acetate-hexane to yield 57 as colorless syrup, 0.106 mg (100%): ¹H NMR: 0.94 (3H, s), 1.19, 1.21 (6H, s each, Me₂COH), 1.23, 1.35 and 1.24, 1.37 (6H, s each, major and minor 5,5-dimethyloxolane diastereomer), 1.1-1.7 (18H, m), 1.7-1.9 (5H, m), 1.9-2.0 (2H, m), 3.65 (1H, m), 3.81 (3H, s), 4.08 (1H, brs), 5.78 and 5.96 (1H, s each, major and minor acetal diastereomer), 6.89 (2H, m), 7.41 (2H, m).

### [1R,3aR,7aR]-1-(5-Hydroxy-1(S)-{2-[2-(4-methoxy-phenyl)-5,5-dimethyl-[1,3]dioxolan-4(R)-yl]-ethyl}-5-methyl-hexyl)-7a-methyl-octahydro-inden-4-one (58)

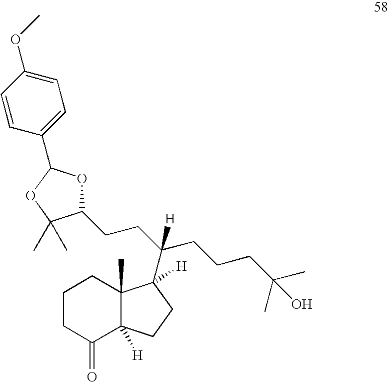

Pyridinium dichromate (230 mg, 0.61 mmol) was added to a stirred mixture containing 57 (0.0838, 0.167 mmol), Celite (185 mg), and dichloromethane (4 mL). The conversion of 57 (Rf 0.31) to 58 (Rf 0.42) was monitored by TLC (1:25 methanol-chloroform) The mixture was diluted with dichloromethane (10 mL) after 2.5 h, then filtered through a layer of silica gel. Filtrate and washings (1:1 dichloromethane-ethyl acetate) were evaporated and the residue:chromatographed (1:4 ethyl acetate-hexane) to give ketone 58, 0.0763 g, 91%: ¹H NMR: 0.63 (3H, s), 1.19, 1.21 and 1.23 (6H, s each, Me₂COH), 1.25, 1.36, 1.38 (6H, m,s,s, 5,5-dimethyloxolane diastereomer), 1.1-1.9 (18H, m), 1.9-2.1 (3H, m), 2.1-2.4 (2H, m), 2.45 (1H, m), 3.66 (1H, m), 3.802 and 3.805 (3H, s each), 5.78 and 5.95 (1H, s each, major and minor acetal diastereomer), 6.89 (2H, m), 7.39 (2H, m).

### [1R,3aR,7aR]-1-[4(R), 5-Dihydroxy-1(S)-(4-hydroxy-4-methyl-pentyl)-5-methyl-hexyl]-7a-methyl-octahydro-inden-4-one (59)

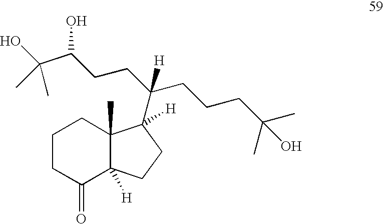

The ketone 58 was stirred in a 1 N oxalic acid solution in 90% methanol. The mixture became homogeneous after a few min. TLC (ethyl acetate) suggested complete reaction after 75 min (Rf 0.24 for 59). Thus, calcium carbonate (0.60 g) was added and the suspension stirred overnight, then filtered. The filtrate was evaporated and flash-chromatographed using a stepwise gradient of 4:1:5 dichloromethane-ethyl acetate-hexane, 1:1 ethyl acetate-hexane, and neat ethyl acetate produce 59 as a colorless residue, 0.060 mg, 94%: ¹H NMR: 0.5 (3H, s), 1.17 (3H, s), 1.22 (6H, s), 1.23 (3H, s), 1.2-1.21 (23H, m), 2.15-2.35 (2H, m), 2.45 (1H, dd, J=7 and 11 Hz), 3.30, 1H, brd).

### [1R,3aR,7aR]-7a-Methyl-1-[5-methyl-1(S)-(4-methyl-4-triethylsilanyloxy-pentyl)-4(R), 5-bis-triethylsilanyloxy-hexyl]-octahydro-inden-4-one (60)

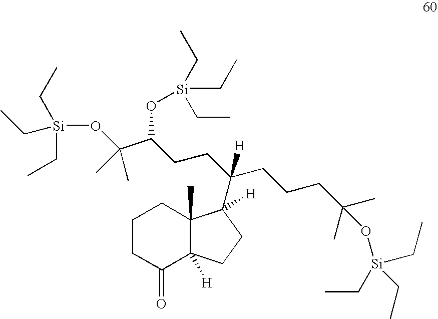

A mixture of 59 (0.055 g, 0.143 mmol), imidazole, (14.9 mg, 1.69 mmol), N,N-dimethylpyridine (6 mg), triethylchlorosilane (0.168 mL, 1 mmol) and N,N-dimethylformamide (1.5 mL) was stirred for 17 h. The reaction was followed by TLC (1:4 ethyl acetate-hexane) and showed rapid conversion to the disilyl intermediate (Rf 0.47). Further reaction proceeded smoothly overnight to give the fully silylated 60 (Rf 0.90). The solution was equilibrated with water (3 mL), equilibrated with ethyl acetate (20 mL), the ethyl acetate layer was washed with water (3×4 mL), dried and evaporated. The residue was flash-chromatographed using a stepwise gradient of hexane and 1:100 ethyl acetate-hexane to yield 60 as a colorless syrup, 0.0813 g, 78.4%: ¹H NMR δ 0.55-0.64 (21H, m), 0.92-0.97 (27H, m), 1.12 (3H, s), 1.18 (3H, s), 1.19 (3H, s), 1.21 (3H, s), 1.1-1.7 (18H, m), 1.9-2.15 (2H, m), 2.15-2.35 (2H, m), 2.43 (1H, dd, J=7.7 and 11 Hz), 3.30 (1H, dd, J=3 and 8.4 Hz).

### [1R,3aR,7aR,4E]-4-{2(Z)-[3(S),5(R)-Bis-(tert-butyl-dimethyl-silanyloxy)-2-methylene-cyclohexylidene]-ethylidene}-7a-methyl-1-[5-methyl-1(S)-(4-methyl-4-triethylsilanyloxy-pentyl)-4(R),5-bis-triethylsilanyloxy-hexyl]-octahydro-indene (61)

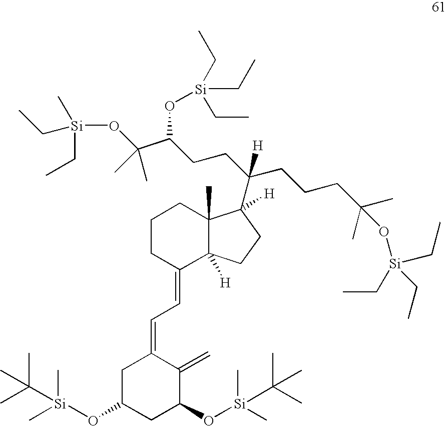

A solution of 1.6 M butyllithium in hexane (0.14 mL) was added to a solution of phosphine (0.1308 g, 0.224 mmol) in tetrahydrofuran (1.5 mL) at −70° C. After 10 min a solution of ketone 60 (0.0813 g, 0.112 mmol) in tetrahydrofuran (1.5 mL) was added dropwise over a 15 min period. The yield color had faded after 3 h so that pH 7 phosphate buffer (2 mL) was added and the temperature allowed to increase to 0° C. The mixture was equilibrated with hexane (30 mL), the organic layer was washed with brine (5 mL), dried and evaporated to give a colorless oil that was purified by flash-chromatography (1:100 ethyl acetate-hexane). Only the band with Rf 0.33 (TLC 1:39 ethyl acetate-hexane) was collected. Evaporation of those fractions gave 61 as colorless syrup, 0.070 g, 57%: ¹H NMR δ 0.06 (12H, brs), 0.53-4.64 (21H, m), 0.88 (18H, s), 0.92-0.97 (27H, m), 1.11 (3H, s), 1.177 (3H, s), 1.184 (3H, s), 1.195 (3H, s), 1-1.9 (22H, m), 1.98 (2H, m), 2.22 (1H, m), 2.45 (1H, m), 2.83 (1H, brd, J=13 Hz, 3.27 (1H, d, J=6 Hz), 4.19 (1H, m), 4.38 (1H, m), 4.87 (1H, brs), 5.18 (1H, brs), 6.02 (1H, d, J=11 Hz), 6.24 (1H, d, J=11 Hz).

### Synthesis of 1,25-Dihydroxy-21 (2R,3-dihydroxy-3-methyl-butyl)-20S-Cholecalciferol (50)

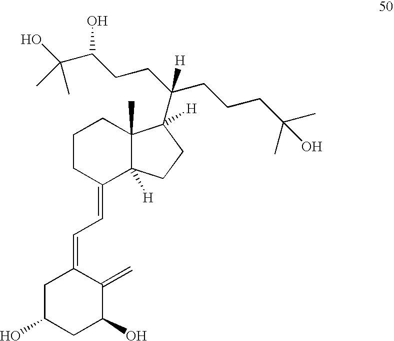

The deprotection reaction of 61 (0.068 g, 0.06238 mmol) in 1M solution of tetrabutylammonium fluoride in tetrahydrofuran, followed by TLC (ethyl acetate), gradually proceeded to give 50 (Rf 0.19). The mixture was diluted with brine (5 mL) after 25 h, stirred for 5 min the equilibrated with ethyl acetate (35 mL) and water (15 mL). The aqueous layer was re-extracted once with ethyl acetate (35 mL), the combined extracts were washed with water (5×10 mL) and brine (5 mL) then dried and evaporated. The residue was flash-chromatographed using a linear gradient of 1:1 and 2:1 ethyl acetate-hexane, and 2:98 methanol-ethyl acetate to give a residue that was taken up in methyl formate and evaporated to a white foam, 30 mg, 93%: [α]_{D}+29.3° (methanol, c 0.34); MHz ¹H NMR δ: 0.55 (3H, s), 1.16 (3H, s), 1.21 (9H, s), 1.1-1.75 (22H, m), 1.80 (2H, m), 1.9-2.1 (5H, m), 2.31 (1H, dd, J=7 and 13 Hz), 2.60 (1H, brd), 284 (1H, m), 3.29 (1H, d, J=9.5 Hz), 4.22 (1H, m), 4.43 (1H, m), 5.00 (1H, s), 5.33 (1H, s), 6.02 (1H, d, J=11 Hz), 6.02 (1H, d, J=11 Hz); LR-ES(−) m/z: 564 (M+H₂CO2), 563 M−H+ H2CO₂); HR-ES(+) calcd for C₃₂H₅₄O₅+Na: 541.3863; found 541.3854; UVₘₐₓ(ε): 211 (15017), 265 (15850), 204 sh (14127), 245 sh (13747) nm.

### Synthetic Example 43

### Synthesis of 1,25-Dihydroxy-21-(2R,3-dihydroxy-3-methyl-butyl)-20S-19-nor-cholecalciferol (62)

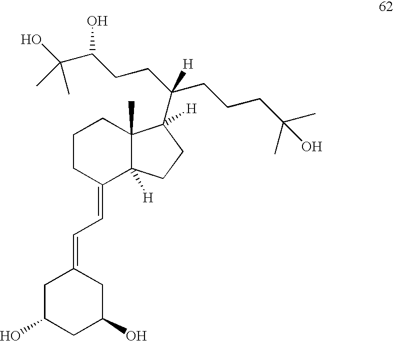

### [1R,3aR,7aR,4E]-4-{2(Z)-[3(S),5(R)-Bis-(tert-butyl-dimethyl-silanyloxy)-cyclohexylidene]-ethylidene}-7a-methyl-1-[5-methyl-1(S)-(4-methyl-4-triethylsilanyloxy-pentyl)-4(R), 5-bis-triethylsilanyloxy-hexyl]-octahydro-indene (63)

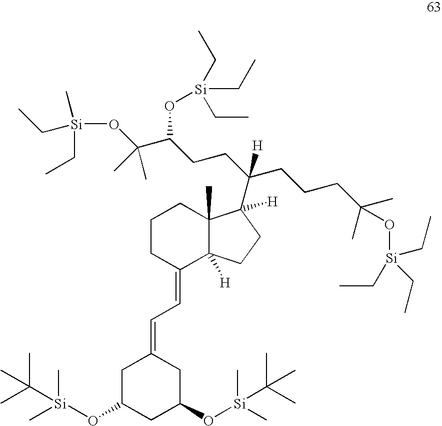

A solution of 1.6 M butyllithium in hexane was added to a solution of phosphine in tetrahydrofuran at −70° C. After 10 min a solution of ketone 60 from Example 2 in tetrahydrofuran was added dropwise over a 15 min period. After the yield color had faded pH 7 phosphate buffer was added and the temperature allowed to increase to 0° C. The mixture was equilibrated with hexane, the organic layer was washed with brine, dried and evaporated to give a colorless oil that was purified by flash-chromatography (1:100 ethyl acetate-hexane) that gave 63.

### 1,25-Dihydroxy-21-(2R,3-dihydroxy-3-methyl-butyl)-20S-19-nor-cholecalciferol (62)

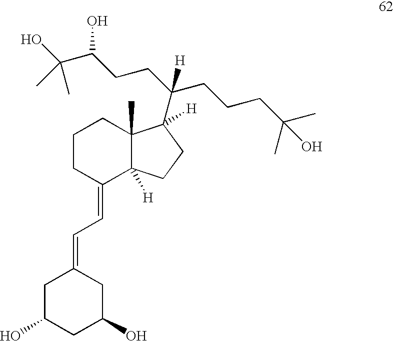

The deprotection reaction of 63 was carried out in 1M solution of tetrabutylammonium fluoride in tetrahydrofuran to give 62. The mixture was diluted with brine after 25 h, stirred for 5 min and then equilibrated with ethyl acetate and water. The aqueous layer was re-extracted once with ethyl acetate, the combined extracts were washed with water and brine, and then dried and evaporated. The residue was flash-chromatographed to give a residue that was taken up in methyl formate and evaporated to yield 62.

### Synthetic Example 44

### Synthesis of 1,25-dihydroxy-20S-21(3-hydroxy-3-methyl-butyl)-24-keto-19-nor-cholecalciferol (64)

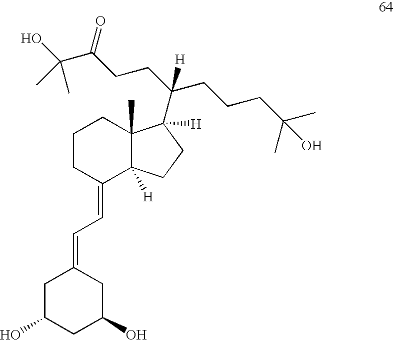

### (R)-6-[(1R,3aR,4S,7aR)-4-(tert-Butyl-dimethyl-silanyloxy)-7a-methyl-octahydro-inden-1-yl]-2-methyl-7-phenylsulfanyl-heptan-2-ol (65)

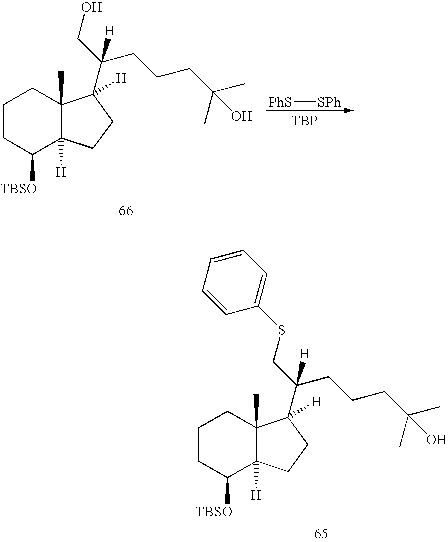

The reaction above was carried out as described in Tet. Lett. 1975, 17: 1409-12. Specifically, a 50 mL round-bottom flask was charged with 1.54 g (3.73 mmol) of (R)-2-[(1R,3aR,4S,7aR)-4-(tert-Butyldimethylsilanyloxy)-7a-methyloctahydroinden-1-yl]-6-methylheptane-1,6-diol (1) (Eur. J. Org. Chem. 2004, 1703-1713) and 2.45 g (11.2 mmol) of diphenylsulfide. The mixture was dissolved in 5 mL of pyridine and 2.27 g (11.2 mmol, 2.80 mL) of tributylphosphine was added. The mixture was stirred overnight and then diluted with 20 mL of toluene and evaporated. The residue was again taken up in toluene and evaporated, the remaining liquid chromatographed on silica gel using stepwise gradients of hexane, 1:39, 1:19 and 1:9 ethyl acetate-hexane to provide the title compound 65 as a syrup, 1.95 g.

### (R)-7-Benzenesulfonyl-6-[(1R,3aR,4S,7aR)-4-(tert-butyl-dimethyl-silanyloxy)-7a-methyl-octahydro-inden-1-yl]-2-methyl-heptan-2-ol (67) and (1R,3aR,4S,7aR)-1-((R)-1-Benzenesulfonylmethyl-5-methyl-5-triethylsilanyloxy-hexyl)-4-(tert-butyl-dimethyl-silanyloxy)-7a-methyl-octahydro-indene (68)

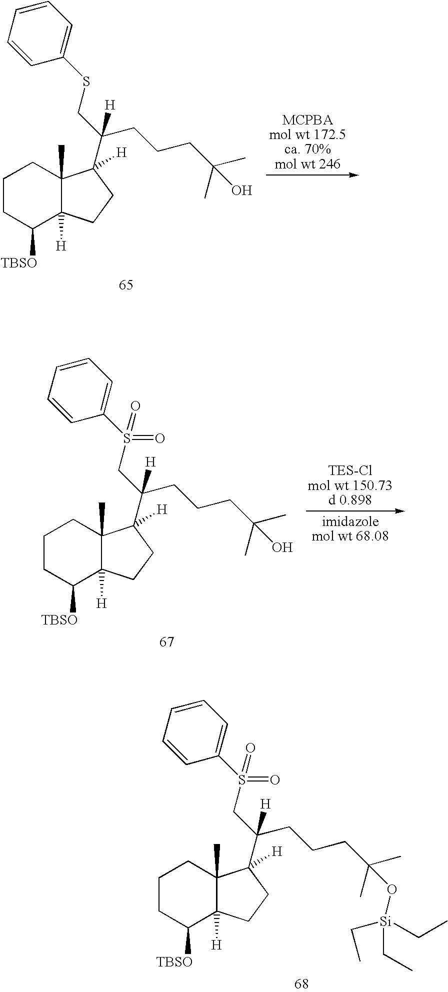

A 500-mL round-bottom flask containing 1.95 g (3.9 mmol) of the crude sulfide 65 was admixed with 84 g of dichloromethane (63 mL). The solution was stirred in an ice bath, then 2.77 g (11 mmol) of meta-chloroperbenzoic acid was added in one portion. The suspension was stirred in the ice bath for 40 min then at room temperature for 2 h. The reaction was monitored by TLC (1:19 methanol-dichloromethane). At the end of the reaction period, only one spot at Rf 0.45 observed. Then, 1.68 g (20 mmol) of solid sodium hydrogen carbonate was added to the suspension, the suspension was stirred for 10 min, then 30 mL of water was added in portions and vigorous stirring continued for 5 min to dissolve all solids. The mixture was further diluted with 40 mL of hexane, stirred for 30 min, transferred to a separatory funnel with 41.6 g of hexane. The lower layer was discarded and the upper one was washed with 25 mL of saturated sodium hydrogen carbonate solution, dried (sodium sulfate) and evaporated to give 3.48 g of 67. This material was triturated with hexane, filtered, and evaporated, to leave 67 as a cloudy syrup (2.81 g) that was used directly in the next step.

A 100-mL round bottom flask containing 2.81 g of 67 obtained above, was charged with 30 mL of N,N-dimethylformamide 1.43 g of (21 mmol) of imidazole and 1.75 mL of (10 mmol) of triethylsilyl chloride. The mixture was stirred for 17 h then diluted with 50 g of ice-water, stirred for 10 min, further diluted with 5 mL of brine and 60 mL of hexane. The aqueous layer was re-extracted with 20 mL of hexane, both extracts were combined, washed with 2×30 mL of water, dried, evaporated. This material contained a major spot with Rf 0.12 (1:39 ethyl acetate-hexane) and a minor spot with Rf 0.06. This material was chromatographed on silica gel using hexane, 1:100, 1:79, 1:39 and 1:19 ethyl acetate-hexane as stepwise gradients. The major band was eluted with 1:39 and 1:19 ethyl acetate-hexane to yield 1.83 g of 68.

### (R)-5-Benzenesulfonyl-6-[(1R,3aR,4S,7aR)-4-(tert-butyl-dimethyl-silanyloxy)-7a-methyl-octahydro-inden-1-yl]-10-methyl-2-(R)-methyl-10-triethylsilanyloxy-undecane-2,3-diol (69)

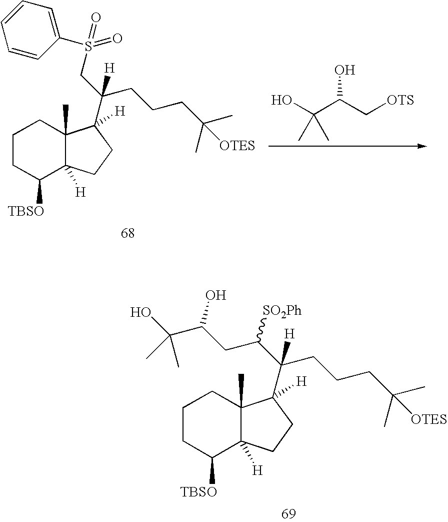

A 100-mL 3-neck round-bottom flask, equipped with magnetic stirrer, thermometer and Claisen adapter with rubber septum and nitrogen sweep, was charged with 1.7636 g of (2.708 mmol) of sulfone 68, 1.114 g of (4.062 mmol) tosylate, and 50 mL of tetrahydrofuran freshly distilled from benzophenone ketyl. This solution was cooled to −20° C. and 9.31 mL of a 1.6 M butyllithium solution in hexane was added dropwise at <−20° C. The temperature range between −10 and −20° C. was maintained for 5 h. The cooling bath was removed and 50 mL of saturated ammonium chloride solution added followed by 75 mL of ethyl acetate and enough water to dissolve all salts. The organic layer was washed with 15 mL of brine, dried, and evaporated to a colorless oil. This residue was chromatographed on silica gel using hexane, 1:9, 1:6, 1:4 and 1:3 ethyl acetate-hexane as stepwise gradients. The main band was eluted with 1:4 and 1:3 ethyl acetate-hexane to furnish 1.6872 g of compound 69 as colorless syrup.

### (S)-6-[(1R,3aR,4S,7aR) 4-(tert-Butyl-dimethyl-silanyloxy)-7a-methyl-octahydro-inden-1-yl]-10-methyl-2-(R)-methyl-10-triethylsilanyloxy-undecane-2,3-diol (70)

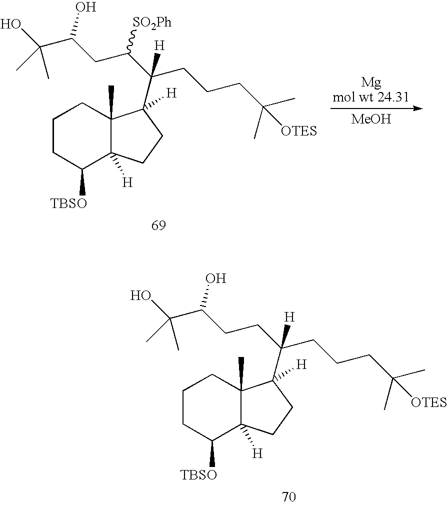

A 25-mL 2-neck round-bottom flask, equipped with magnetic stirrer, thermometer and Claisen adapter with rubber septum and nitrogen sweep, was charged with 1.6872 g (2.238 mmol) of sulfone 69 and 40 mL of methanol. Then 1.25 g (51.4 mmol) of magnesium was added to the stirred solution in two equal portions, in a 30 min time interval. The suspension was stirred for 70 min then another 0.17 g of magnesium and ca. 5 mL of methanol was added and stirring continued 1 h. The mixture was then diluted with 100 mL of hexane and 50 mL of 1 M sulfuric acid was added dropwise to give two liquid phases. The aqueous layer was neutral. The aqueous layer was re-extracted once with 25 mL of 1:1 dichloromethane-hexane. The organic layers were combined then washed once with 15 mL of brine, dried and evaporated. The resulting material was chromatographed on silica gel using hexane, 1:39, 1:19 and 1:9 ethyl acetate-hexane as stepwise gradients. The main band was eluted with 1:9 ethyl acetate-hexane to provide 1.2611 g of 70 as a colorless syrup.

### (S)-6-[(1R,3aR,4S,7aR)-4-(tert-Butyl-dimethyl-silanyloxy)-7a-methyl-octahydro-inden-1-yl]-2,10-dihydroxy-2,10-dimethyl-undecan-3-one (71)

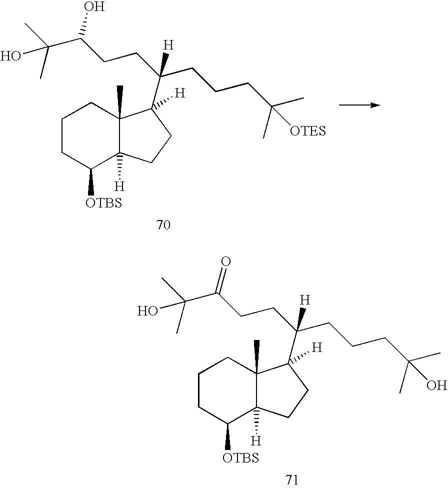

A 25-mL round-bottom flask, equipped with magnetic stirrer, thermometer, Claisen adapter with nitrogen sweep and rubber septum, was charged with 518 mg (3.88 mmol) of N-chlorosuccinamide and 11 mL of toluene. Stir for 5 min (not all dissolved), then cool to 0° C. and add 2.4 mL (4.8 mmol) of a 2M dimethyl sulfide solution in toluene. The mixture was stirred from 5 min then cooled to −30° C. and a solution of 0.7143 g (1.165 mmol) of the diol 70 in 4×1.5 mL of toluene was added dropwise at −30° C. Stirring was continued at this temperature for 1 h. The mixture was then allowed to warm to −10° C. during a 2 h time period then cooled to −17° C. and 3.20 mL (6.4 mmol) of 2 M triethylamine in toluene added dropwise. The mixture was stirred at −17 to −20° C. for 10 min then allowed to warm to room temperature slowly. The mixture was chromatographed on a silica gel column using hexane, 1:79, 1:39, 1:19, 1:9, 1:4, and 1:1 ethyl acetate-hexane as stepwise gradients. The major band was eluted with 1:1 ethyl acetate-hexane providing 0.3428 g of the compound 71 as solids.

### (S)-2,10-Dihydroxy-6-((1R,3aR,4S,7aR)-4-hydroxy-7a-methyl-octahydro-inden-1-yl)-2,10-dimethyl-undecan-3-one (72)

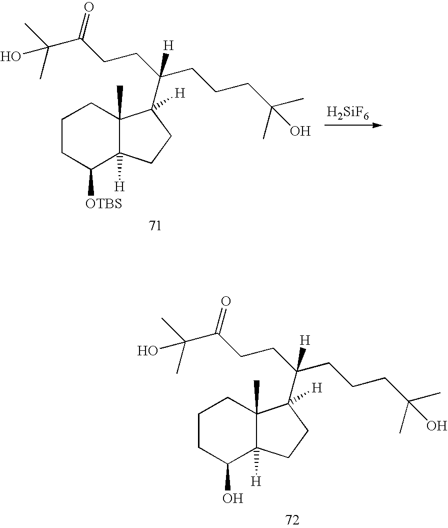

A 25-mL round-bottom flask, equipped with magnetic stirrer was charged with 0.3428 g (0.69 mmol) of the diol 71, was dissolved in 5 mL of acetonitrile then 1.25 mL of fluorosilicic acid solution. After 3 h, the mixture was distributed between 35 mL of ethyl acetate and 10 mL of water, the aqueous layer was re-extracted with 10 mL of ethyl acetate, the organic layers combined, washed with 2×5 mL of water, once with 5 mL of 1:1 brine-saturated sodium hydrogen carbonate solution, dried and evaporated. This material was chromatographed on silica gel using 1:4, 1:3, 1:2, and 1:1 as stepwise gradients furnishing 0.2085 g of the title compound 72.

### (1R,3aR,7aR)-1-[(S)-5-Hydroxy-1-(4-hydroxy-4-methyl-pentyl)-5-methyl-4-oxo-hexyl]-7a-methyl-octahydro-inden-4-one (73)

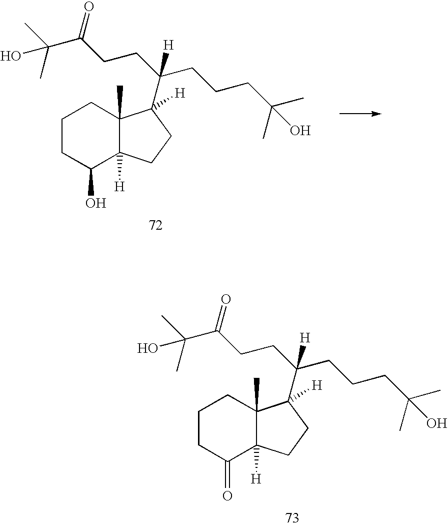

A 25-mL round bottom flask was charged with 0.2153 g (0.56 mmol) of 72, 5 mL of dichloromethane, and 0.20 g of Celite. To this stirred suspension was added, in on portion, 1.00 g (2.66 mmol) of pyridinium dichromate. The reaction stirred for 3 h and the progress was monitored by TLC (1:1 ethyl acetate-hexane). The reaction mixture was diluted with 5 mL of cyclohexane then filtered trough silica gel G. The column was eluted with dichloromethane followed by 1:1 ethyl acetate-hexane until no solute was detectable in the effluent. The effluent was evaporated and the colorless oil. This oil was then chromatographed on a silica gel using 1:4, 1:3, 1:2, 1:1 and 2:1 ethyl acetate-hexane as stepwise gradients to furnish 0.2077 g of the diketone 73.

### (1R,3aR,7aR)-7a-Methyl-1-[(S)-5-methyl-1-(4-methyl-4-trimethylsilanyloxy-pentyl)-4-oxo-5-trimethylsilanyloxy-hexyl]-octahydro-inden-4-one (4)

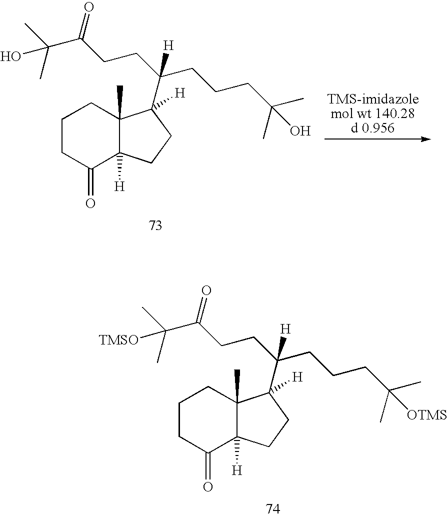

A 25-mL round bottom flask was charged with 0.2077 g (0.545 mmol) of the diketone 73. This material was dissolved in a mixture of 0.5 mL of tetrahydrofuran and 3 mL of cyclohexane. To the resulting mixture was added 0.30 mL (2.0 mmol) of TMS-imidazole. The reaction mixture was diluted with 3 mL of hexane after 10 h then concentrated and chromatographed on silica gel using hexane, 1:79, 1:39, 1:19 and ethyl acetate-hexane as stepwise gradients to provide 0.2381 g of 74 as a colorless oil.

### (S)-6-((1R,3aS,7aR)-4-2-[(R)-3-((R)-tort-Butyldimethylsilanyloxy)-5-(tert-butyldimethylsilanyloxy)-cyclohexylidene]-ethylidene)-7a-methyloctahydroinden-1-yl)-2,10-dimethyl-2,10-bis-trimethylsilanyloxyundecan-3-one (75)

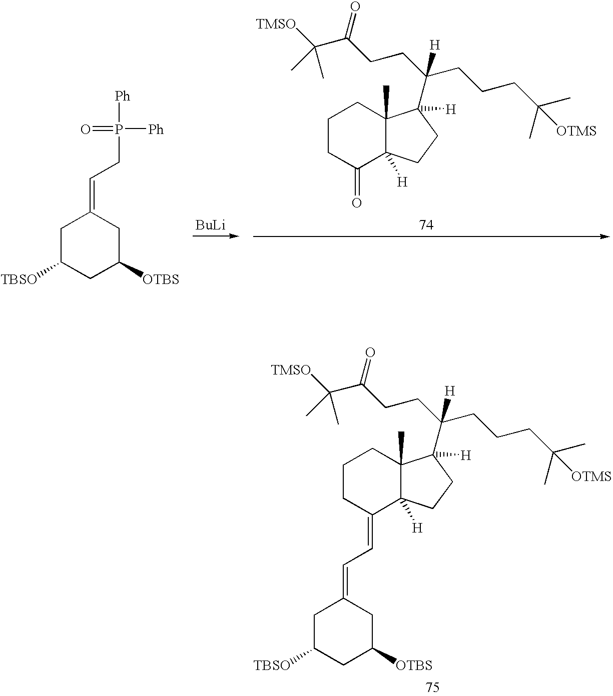

A 15-mL 3-neck pear-shaped flask, equipped with magnetic stirrer, thermometer and a Claisen adapter containing a nitrogen sweep and rubber septum, was charged with 0.2722 g (0.4768 mmol) of [2-[(3R,5R)-3,5-bis(tert-butyldimethylsilanyloxy)cyclohexylidene]ethyl]diphenylphosphine oxide and 2 mL of tetrahydrofuran. The solution was cooled to −70° C. and 0.30 mL of 1.6 M butyllithium in hexane was added. The deep red solution was stirred at that temperature for 10 min then 0.1261 g (0.240 mmol) of the diketone 74, dissolved in 2 mL of tetrahydrofuran was added, via syringe, dropwise over a 10 min period. After 3 h and 15 min, 5 mL of saturated ammonium chloride solution was added at −65° C., the mixture allowed to warm to 10° C. then distributed between 35 mL of hexane and 10 mL of water. The aqueous layer was re-extracted once with 10 mL of hexane, the combined layers washed with 5 ml of brine containing 2 mL of pH 7 buffer, then dried and evaporated. This material was chromatographed on a flash column, 15×150 mm using hexane and 1:100 ethyl acetate-hexane as stepwise gradients to yield 0.1572 g of the title compound 75 as a colorless syrup.

### 1,25-Dihydroxy-20S-21 (3-hydroxy-3-methyl-butyl)-24-keto-19-nor-cholecalciferol (64)

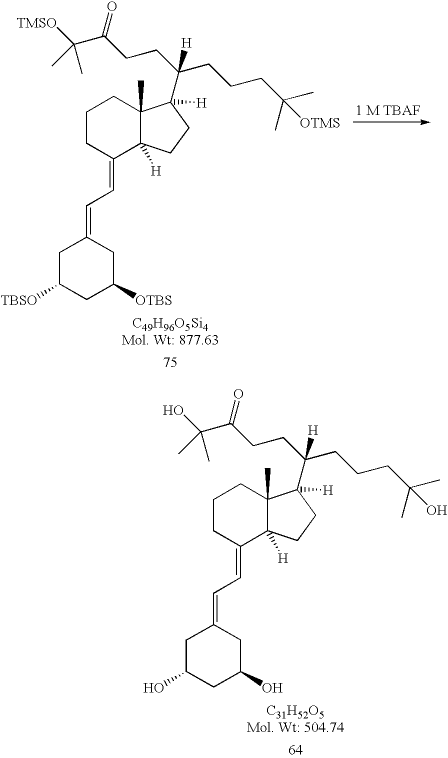

A 15-mL 3-neck round-bottom flask, equipped with magnetic stirrer, was charged with 155 mg (0.17 mmol) of tetrasilyl ether 75. This colorless residue was dissolved is 2 mL of a 1M solution of tetrabutylammonium fluoride in tetrahydrofuran. After 43 h an additional 0.5 mL of 1M solution of tetrabutylammonium fluoride solution was added and stirring continued for 5 h. The light-tan solution was the diluted with 5 mL of brine, stirred for 5 min and transferred to a separatory funnel with 50 mL of ethyl acetate and 5 mL of water then re-extraction with 5 mL of ethyl acetate. The organic layers were combined, washed with 5×10 mL of water, 10 mL of brine, dried and evaporated. The resulting residue was chromatographed on a 15×123 mm column using 2:3, 1:1, 2:1 ethyl acetate-hexane, and ethyl acetate as stepwise gradients to provide the 64 as a white solid (TLC, ethyl acetate, Rf 0.23) that was taken up in methyl formate, filtered and evaporated furnishing 0.0753 g of the title compound 64 as a solid substance.

### Synthetic Example 45

### Synthesis of 1,25-dihydroxy-20S-21(3-hydroxy-3-methyl-butyl)-24-keto-cholecalciferol (76)

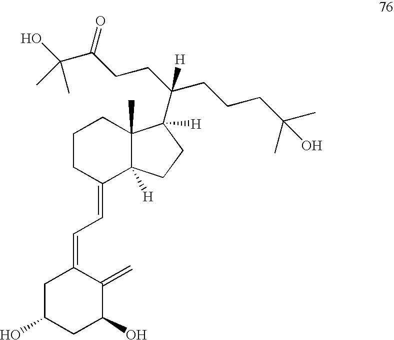

### (S)-6-{(1R,3aS,7aR)-4-[2-[(R)-3-tert-Butyl-dimethyl-silanyloxy)-5-((S)-tert-butyl-dimethyl-silanyloxy)-2-methylene-cyclohexylidene]-eth-(E)-ylidene]-7a-methyl-octahydro-inden-1-yl}-2,10 dimethyl-2,10-bis-trimethylsilanyloxy-undecan-3-one (77)

Compound 77 was prepared as described for 75 in Example 4 but by reacting 74 with [(2Z)-2-[(3S,5R)-3,5-bis(tert-butyldimethylsilanyloxy)methylenecyclohexylidene]-ethyl]diphenylphosphine oxide.

### 1,25-Dihydroxy-20S-21 (3-hydroxy-3-methyl-butyl)-24-keto-cholecalciferol (76)

Compound 76 was prepared from 77 by deprotecting 77 as described in Example 44 for 64.

### Synthetic Example 46

### Synthesis of 1α,25-Dihydroxy-16-ene-20-cyclopropyl-cholecalciferol (78)

Compound (78) was synthesized according to the following synthetic procedure.

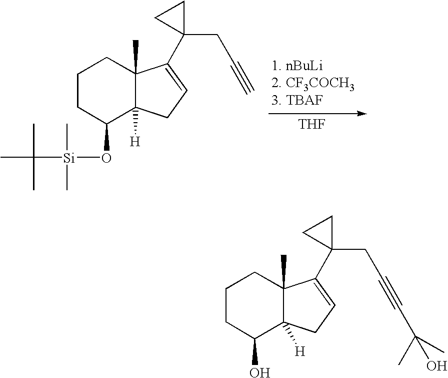

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])cyclopropyl}-ethynyl (1.0 g, 2.90 mmol) in tetrahydrofurane (15 mL) at −78° C. was added n-BuLi (2.72 mL, 4.35 mmol, 1.6M in hexane). After stirring at −78° C. for 1 h., acetone (2.5 mL, 34.6 mmol) was added and the stirring was continued for 2.5 h. NH₄Cl_{aq} was added (15 mL) and the mixture was stirred for 15 min at room temperature then extracted with AcOEt (2×50 mL). The combined extracts were washed with brine (50 mL) and dried over Na₂SO₄. The residue after evaporation of the solvent (2.4 g) was purified by FC (50 g, 10% AcOEt in hexane) to give (3aR,4S,7aR)-5-{1-[4-(tert-Butyl-dimethyl-silanyloxy)-7a-methyl-3a,4,5,6,7,7a-hexahydro-3H-inden-1-yl]-cyclopropyl}-2-methyl-pent-3-yn-2-ol (1.05 g, 2.61 mmol) which was treated with tetrabutylammonium fluoride (6 mL, 6 mmol, 1.0M in THF) and stirred at 65-75° C. for 48 h. The mixture was diluted with AcOEt 25 mL) and washed with water (5×25 mL), brine (25 mL). The combined aqueous washes were extracted with AcOEt (25 mL) and the combined organic extracts were dried over Na₂SO₄. The residue after evaporation of the solvent (1.1 g) was purified by FC (50 g, 20% AcOEt in hexane) to give the titled compound (0.75 g, 2.59 mmol, 90%). [α]³⁰_{D}=+2.7 c 0.75, CHCl₃. ¹H NMR (CDCl₃): 5.50 (1H, m), 4.18 (1H, m), 2.40 (2H, s), 2.35-1.16 (11H, m), 1.48 (6H, s), 1.20 (3H, s), 0.76-0.50 (4H, m); ¹³C NMR (CDCl₃): 156.39, 125.26, 86.39, 80.19, 69.21, 65.16, 55.14, 46.94, 35.79, 33.60, 31.67, 29.91, 27.22, 19.32, 19.19, 17.73, 10.94, 10.37;

MS HREI Calculated for C₂₂H₂₈O₂ M+ 288.2089 Observed M+ 288.2091.

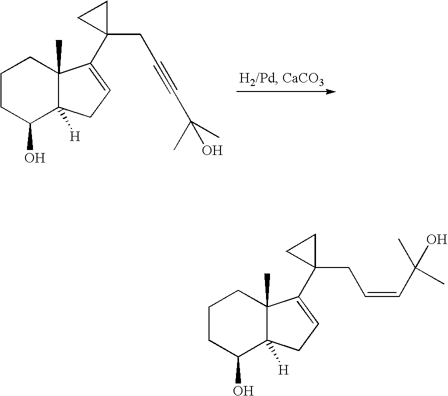

The mixture of (3aR,4S,7aR)-7a-Methyl-1-[1-(4-hydroxy-4-methyl-pent-2-ynyl)-cyclopropyl]-3a,4,5,6,7,7a-hexahydro-3H-inden-4-ol (0.72 g, 2.50 mmol), ethyl acetate (10 mL), hexane (24 mL), absolute ethanol (0.9 mL), quinoline (47 μL) and Lindlar catalyst (156 mg, 5% Pd on CaCO₃) was hydrogenated at room temperature for 2 h. The reaction mixture was filtered through a celite pad and the pad was washed with AcOEt. The filtrates and the washes were combined and washed with 1M HCl, NaHCO₃ and brine. After drying over Na₂SO₄ the solvent was evaporated and the residue (0.79 g) was purified by FC (45 g, 20% AcOEt in hexane) to give the titled compound (640 mg, 2.2 mmol, 88%).

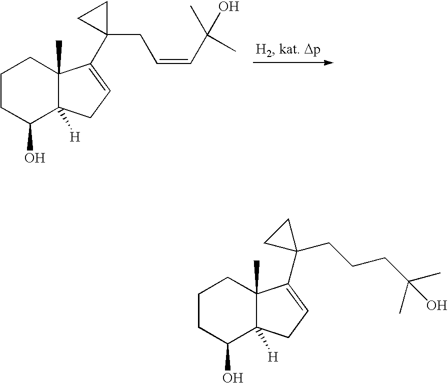

The mixture of (3aR,4S,7aR)-7a-Methyl-1-[1-(4-hydroxy-4-methyl-pent-2Z-enyl)-cyclopropyl]-3a,4,5,6,7,7a-hexahydro-3H-inden-4-ol (100 mg, 0.34 mmol), 1,4-bis(diphenyl-phosphino)butane 1,5 cyclooctadiene rhodium tetrafluoroborate (25 mg, 0.034 mmol), dichloromethane (5 mL) and one drop of mercury was hydrogenated using Paar apparatus at room temperature and 50 p.s.i. pressure for 3 h. The reaction mixture was filtered through Celite pad, which was then washed with ethyl acetate. The combine filtrates and washes were evaporated to dryness (110 mg) and purified by FC (10 g, 20% AcOEt in hexane) to give the titled compound (75 mg, 0.26 mmol, 75%). [α]³⁰_{D}=−8.5 c 0.65, CHCl₃. ¹H NMR (CDCl₃): 5.37 (1H, m,), 4.14 (1H, m), 2.37-1.16 (17H, m), 1.19 (6H, s), 1.18 (3H, s), 0.66-0.24 (4H, m); MS HREI Calculated for C₁₉H₃₂O₂ M+H 292.2402. Observed M+H 292.2404.

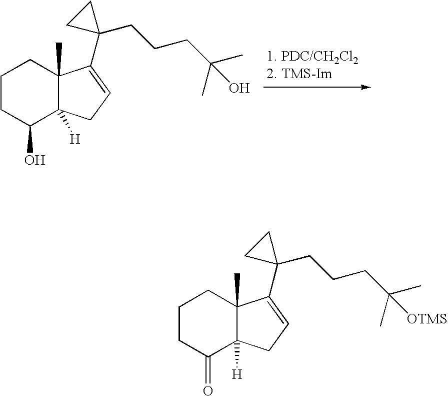

To a stirred suspension of (3aR,4S,7aR)-7a-Methyl-1-[1-(4-hydroxy-4-methyl-pentenyl)-cyclopropyl]-3a,4,5,6,7,7a-hexahydro-3H-inden-4-ol (440 mg, 1.50 mmol) and Celite (2.0 g) in dichloromethane (10 mL) at room temperature wad added pyridinium dichromate (1.13 g, 3.0 mmol). The resulting mixture was stirred for 5 h filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pentenyl) cyclopropyl]-3a,4,5,6,7,7a-hexahydro-3H-inden-4-one (426 mg, 1.47 mmol, 98%). To a stirred solution of (3aR,7aR)-7a-Methyl-1-[1-(4-hydroxy methyl-pentenyl)-cyclopropyl]-3a,4,5,6,7,7a-hexahydro-3H-inden-4-one (424 mg, 1.47 mmol) in dichloromethane (10 mL) at room temperature was added trimethylsilyl-imidazole (0.44 mL, 3.0 mmol). The resulting mixture was stirred for 1.0 h filtered through silica gel (10 g) and the silica gel pad was washed with 10% AcOEt in hexane. Combined filtered and washes were evaporated to give the titled compound (460 mg, 1.27 mmol, 86%). [α]²⁹_{D}=−9.9 c 0.55, CHCl₃. ¹H NMR (CDCl₃): 5.33 (1H, dd, J=3.2, 1.5 Hz), 2.81 (1H, dd, J=10.7, 6.2 Hz), 2.44 (1H, ddd, J=15.6, 10.7, 1.5 Hz), 2.30-1.15 (13H, m) overlapping 2.03 (ddd, J=15.8, 6.4, 3.2 Hz), 1.18 (6H, s), 0.92 (3H, s), 0.66-4.28 (4H, m), 0.08 (9H, s); ¹³C NMR (CDCl₃): 211.08 (0), 155.32 (0), 124.77(1), 73.98 (0), 64.32 (1), 53.91 (0), 44.70 (2), 40.45 (2), 38.12 (2), 34.70 (2), 29.86 (3), 29.80 (3), 26.80 (2), 24.07 (2), 22.28 (2), 21.24 (0), 18.35 (3), 12.60 (2), 10.64 (2), 2.63 (3); MS HRES Calculated for C₂₂H₃₈O₂Si M+ 362.2641. Observed M+ 362.2648.

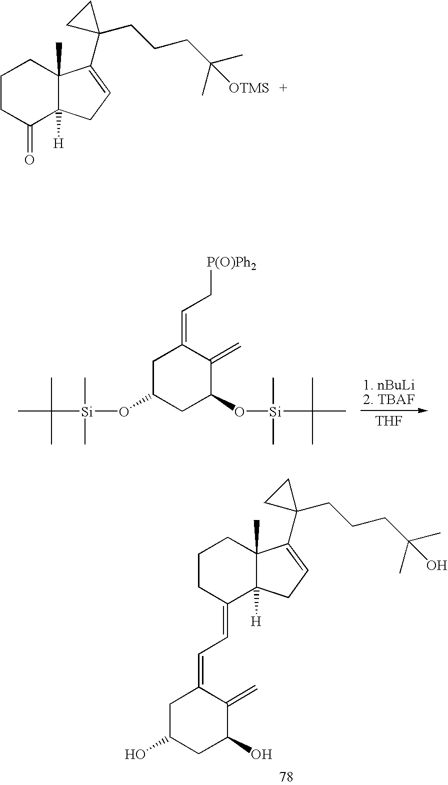

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (675 mg, 1.16 mmol) in tetrahydrofurane (8 mL) at −78° C. was added n-BuLi (0.73 mL, 1.17 mmol). The resulting mixture was stirred for 15 min and solution of (3aR,7aR)-7a-Methyl-1-[1-(4-methyl-4-trimethylsilanyloxy-pentyl)-cyclopropyl]-3a,4,5,6,7,7a-hexahydro-3H-inden-4-one (210 mg, 0.58 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (850 mg) after evaporation of the solvent was purified by FC (15 g, 10% AcOEt in hexane) to give 1α,3β-Di(tert-Butyl-dimethyl-silanyloxy)-25-trimethylsilanyloxy-16-ene-20-cyclopropyl-cholecalciferol (382 mg, 0.53 mmol). To the 1α,3β-Di(tert-Butyl-dimethyl-silanyloxy)-25-trimethylsilanyloxy-16-ene-20-cyclopropyl-cholecalciferol (382 mg, 0.53 mmol) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (380 mg) after evaporation of the solvent was purified by FC (15 g, 50% AcOEt in hexane and AcOEt) to give the titled compound (78) (204 mg, 0.48 mmol, 83%). [α]²⁹_{D}=+ 16.1 c 0.36, EtOH. UV λmax (EtOH): 208 nm (ε 17024), 264 nm (ε 16028); ¹H NMR (CDCl₃): 6.37 (1H, d, J=11.3 Hz), 6.09 (1H, d, J=11.1 Hz), 5.33 (2H, m), 5.01 (1H, s), 4.44 (1H, m), 4.23 (1H, m), 2.80 (1H, m), 2.60 (1H, m), 2.38-1.08 (20H, m), 1.19 (6H, s), 0.79 (3H, s), 0.66-0.24 (4H, m); ¹³C NMR (CDCl₃): 157.07(0), 147.62 (0), 142.49 (0), 133.00 (0), 124.90(1), 124.73(1), 117.19 (1), 111.64 (2), 71.10 (1), 70.70 (0), 66.88 (1), 59.53 (1), 50.28 (0), 45.19 (2), 43.85 (2), 42.86 (2), 38.13 (2), 35.59 (2), 29.27 (2), 29.14 (3), 28.65(2), 23.57 (2), 22.62 (2), 21.29 (0), 17.84(3), 12.74(2), 10.30(2); MS HRES Calculated for C₂₈H₄₂O₃ M+Na 449.3026. Observed M+Na 449.3023.

### Synthetic Example 47

### Synthesis of 1-alpha-fluoro-25-hydroxy-16,23E-diene-26,27-bishomo-20-epi-cholecalciferol (79) (Compound A)

Compound (79) is synthesized according to the following synthetic procedure.

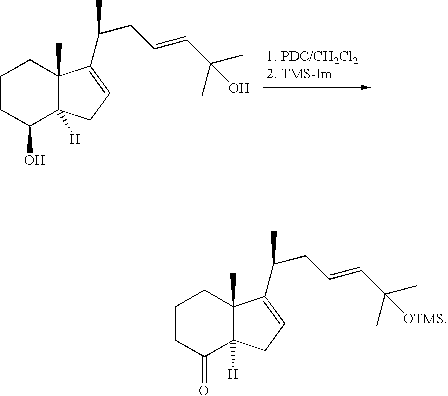

To a stirred suspension of 11-(5-Hydroxy-1,5-dimethyl-hex-3-enyl)-7a-methyl-3a,4,5,6,7,7a-hexahydro-3H-inden-4-ol and Celite in dichloromethane (10 mL) at room temperature is added pyridinium dichromate. The resulting mixture is stirred for 5 h filtered through silica gel, and then silica gel pad is washed with 20% AcOEt in hexane. The combined filtrate and washes are evaporated, to give a ketone. To a stirred solution of ketone in dichloromethane at room temperature is added trimethylsilyl-imidazole. The resulting mixture is stirred for 1.0 h filtered through silica gel and the silica gel pad is washed with 10% AcOEt in hexane. Combined filtered and washes are evaporated to give the titled compound.

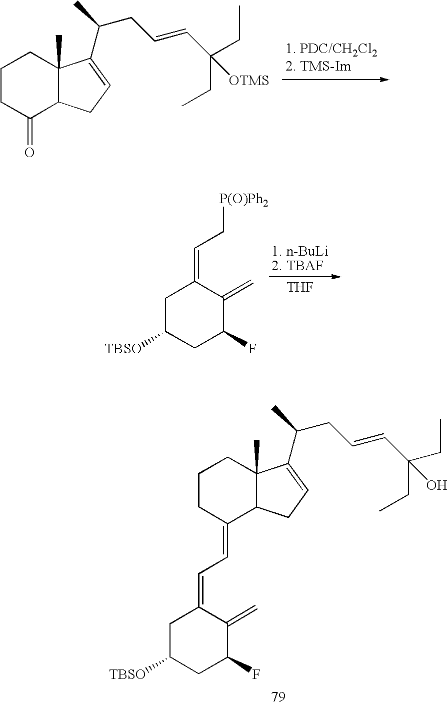

To a stirred solution of a tert-Butyl-{3-[2-(diphenyl-phosphinoyl)-ethylidene]-5-fluoro-4-methylene-cyclohexyloxy}dimethyl-silanein tetrahydrofurane at −78° C. is added n-BuLi. The resulting mixture is stirred for 15 min and solution of 1-(5-Ethyl-1-methyl-5-trimethylsilanyloxy-hept-3-enyl)-7a-methyl-3,3a,5,6,7,7a-hexahydro-inden-4-one in tetrahydrofurane is added dropwise. The reaction mixture is stirred at −78° C. for 3.5 h diluted with hexane washed brine and dried over Na₂SO₄. The residue after evaporation of the solvent was purified by FC (15 g, 10% AcOEt in hexane) to give the silylated compound. To the silylated compound, tetrabutylammonium fluoride is added, at room temperature. The mixture is stirred for 15 h. diluted with AcOEt (25 mL) and washed with water (5×20 mL), brine (20 mL) and dried over Na₂SO₄. The residue (380 mg) after evaporation of the solvent is purified by FC (15 g, 50% AcOEt in hexane and AcOEt) to give the titled compound (79).

### BIOLOGICAL EXAMPLES

### Example 1

### Materials and Methods

### Stromal Cell Preparation

Tissue was gently minced into small pieces (1 to 2 mm³) and incubated at 37° C. for 1 h with 0.1% type A collagenase. At the end of the incubation, single stromal cells were separated from large clumps of epithelium by a 10 min. period of differential sedimentation at unity gravity. The top 8 ml of medium, containing predominantly stromal cells, were then slowly removed and the cells were collected by centrifugation. The stromal-enriched fraction was washed twice in culture medium and allowed to adhere selectively to tissue culture dishes for 15 min.

Thereafter, nonattached epithelial cells still present were removed and a purified stromal preparation was obtained on the surface of the culture dishes.

### Total RNA Extraction

Cells were incubated at 37° C. in 3% FBS DMEM (without Compound A, with Compound A at 1 uM concentration or with Compound A at 0.1 uM concentration) or 10% FBS DMEM (without Compound A, with Compound A at 1 uM concentration or with Compound A at 0.1 uM concentration). After 4 or 8 hours of incubation cells were trypsinized and collected as a cell pellet.

For total RNA extraction it was used the RNeasy Mini Kit QIAGEN (cat. no. 74106) briefly described below.

Cells were distrupted by addition of Buffer RLT and the lysate was loaded onto a QlAshredder spin column (QIAGEN cat. no. 79656) placed in a 2 ml collection tube and centrifuged for 2 min at maximum speed. A volume of 70% ethanol was added to the homogenized lysate. The sample was loaded on an RNeasy mini column placed in a 2 ml collection tube and centrifuged for 15 sec at >10000 rpm. The RNA bound to the column was digested with a DNase treatment. The column was washed with Buffer RW1 and centrifuged for 15 sec at >10000 rpm. The sample was incubated with DNase I mix (RNase-Free Dnase Set QIAGEN cat. no. 79254) at room temperature for 15 min. The RNeasy mini column was washed with Buffer RW1 and transferred into a new 2 ml collection tube. The column was washed twice with Buffer RPE and centrifuged for 15 sec at >10000 rpm; RNase-free water was loaded onto the column and RNA was eluted after centrifugation for 1 min at >10000 rpm. RNA concentration was evaluated by NanoDrop Spectrophotometer

cDNA Synthesis

The cDNA synthesis was performed by using the kit Applied Biosystems TaqMan Reverse Transcription Reagents (Applied Biosystems cat. no. 8080234).

1 ug total RNA was retrotranscribed in a RT mix containing RT Buffer 1×, MgCl₂ 5.5 mM, dNTPs 500 uM, Random Hexamers 2.5 uM, RNase inhibitor 40 U and Multiscribe Reverse Transcription 125 U in 100 ul final volume. The mixture was incubated at room temperature for 10 min followed by 30 min at 48° C.; the cDNA concentration obtained was 10 ng/ul.

### Real Time PCR for Gene Expression Quantification

Real Time PCR was performed by using ABI PRISM 7000 Sequence Detection System (Applied Biosystems). 30 ng cDNA were amplified in a 25 ul volume containing TaqMan Universal PCR Master Mix 1× (Applied Biosystems cat. no. 430-4437) and Assay Mix target gene 1× (Applied Biosystems). The genes analysed included Vitamin D Receptor (VDR), Cytochrome P450 (CYP24), Vascular Endothelial Growth Factor (VEGF), Estrogen Receptor alpha (ERα), Estrogen Receptor beta (ERβ), Progesterone Receptor (PR), Aromatase (CYP19), Cyclooxygenase type 2 (COX-2), Interleukin-8 (IL-8), Tumor Necrosis Factor alpha (TNF α), Caspase-3 (CASP3), Caspase-6 (CASP6), Ki-67 Nuclear Antigen (Ki-67).

Samples were incubated 2 min at 50° C., 10 min at 95° C. and amplified for 40 cycles at 95° C. for 15 sec (denaturation) and at 60° C. for 1 min (annealing/extension). The amount of gene expression was normalized to rRNA 18S gene expression and the comparative CT methods (User Bulletin #2 ABI PRISM 7000 Sequence Detection System) was used for relative quantitation.

### Proliferation of Endometrial Stromal Cells In Vitro

The cells were maintained in DMEM supplemented with 10% Fetal Bovine Serum (SIGMA) and 100 U/ml penicillin, 100 ug/ml streptomycin (GIBCO cat. no. 15140-122).

When the stromal cells were grown to confluence, were washed in PBS and then trypsinized using 1× trypsin/EDTA solution (PromoCell cat. no. C-41002). The cells were seeded at 1×10⁵ cells/ml in 96 well flat bottom plate in DMEM, 5% fetal bovine serum and VDR ligand (Compound A) at different concentrations (1 uM-0.1 nM). After 48-96 hours, the supernatants were harvested and the plates were stored at −80° C. for determination of the proliferation. The proliferation was determined with CyQuant Cell Proliferation Assay (Molecular Probe cat. no. C7026). The plates were thawed at room temperature, and 200 uL of the CyQUANT GR dye/cell lysis buffer were added to each sample well.

The plates were incubated for 2-5 minutes at room temperature, protected from light.

The fluorescence was determined using a fluorescence microplate reader with filters appropriate for ˜480 nm excitation and ˜520 nm emission.

### ELISA Hu-IL8

The IL-8 was detected with Human IL-8 ELISA Set (BD OptEIA BD Biosciences cat. No. 555244)

The plate was coated with 100 ul of capture anti human IL-8 diluted 1:250 in Coating buffer (0.1 M Sodium Carbonate, pH 9.5) and incubated over night at 4° C. After washing, plates were blocked by adding 200 ul of Assay Diluent (PBS with 10% FBS, pH 7.0) for 1 to 2 hours at room temperature. The supernatant was discarded and 100 ul standard (recombinant human IL-8 from 200 pg/ml to 3.1 pg/ml) or sample diluted 1:2 in Assay Diluent was added. Plates were incubated for 2 hours at room temperature. After washing, 100 ul of Detection antibody (Detection Antibody 1:250+SAv-HRP reagent 1:250) was added and incubated 1 hours at room temperature.

Plates were washed and 100 ul of Substrate Solution was added to each well. The colorimetric reaction was blocked with Stop Solution (H₂SO₄ 1M). Optical density was determined at 405 nm using microtiter plate reader.

### In Vivo Model of Endometriosis

Balb/c donor mice were injected with estrogen (Estradiol AMSA; 3 ug/mouse) and one week later were sacrificed and the uterus was removed, the two horns isolated and reduced to small fragments. The fragments derived from the isolated uterine horns were resuspended in saline with ampicillin (1 mg/ml) and then injected into the peritoneum of two recipient Balb/c mice, previously anesthesized, through a 0.5 cm incision in the abdominal wall. Estrogen was injected subcutaneously once a week for two weeks in order to support endometrial growth. Antibiotic (ampicillin 1 mg/ml) was administered on the day of the surgery and on the day after. Four hours after the surgery, one mouse in each pair was injected with test compound (100 ug/kg) and the other with control, ip once a day, 5 days a week for two weeks. After two weeks, mice were given a lethal dose of anesthetic and their abdomen was opened to check for lesion presence. Lesions can be identified as translucid isolated or grouped cysts mainly found on the abdominal wall, on the pancreas, and around the uterus. In some cases the lesions are necrotic. The lesions were carefully removed and put on a glass slide to dry for 48 hours, and then were weighed. In other experiments lesions are transferred to a lysis solution and mRNA isolated for gene expression analysis. For immunohistochemical analysis, lesions were frozen immediately after isolation.

### Results

### Lesion Weight

FIG. 1 illustrates the effect of treatment using 1-alpha-fluoro-25-hydroxy-16,23E-diene-26,27-bishomo-20-epi-cholecalciferol (Compound A) versus treatment using the control (vehicle only). FIG. 1A presents the entire data set for 17 pairs of mice. FIG. 1B presents the data as the percentage of inhibition of lesion growth in treated mice relative to their control partner. FIG. 1C shows the mean for the treated and control groups. Statistical analysis shows a significant reduction in lesion weight for those mice receiving treatment with a vitamin D compound (p=0.0034 for paired and p=0.020 for unpaired t test).

### Proliferation of Endometrial Stromal Cells In Vitro

FIG. 2 shows the levels of cell proliferation observed for treatment with different concentrations of vitamin D compound (Panel A—Eutopic endometrium, Panel B—Ectopic endometrium). Although there is a degree of variation in the results due to the small dataset used, treatment with Compound A leads in general to a reduction in cell proliferation for Eutopic (FIG. 2A) and Ectopic (FIG. 2B) endometrium. FIG. 2B suggests that this effect may occur in a dose dependent manner.

Ideally, treatment with a vitamin D compound leads to a preferential reduction in the proliferation of ectopic cells over the reduction in proliferation of eutopic cells.

Gene Expression Quantification

FIG. 3 shows the expression levels of VDR (Panel A), VEGF (Panel B), Cyp24 (Panel C) and Cyp 19 (Panel D) for untreated, 1 uM Compound A treated and 0.1 uM Compound A treated groups.

A marked upregulation of Cyp24 expression can be seen in FIG. 3C. Little or no change in the expression of VDR, VEGF or Cyp19 is observed.

Effect of Vitamin D Compounds

It can be dearly seen that in an in vivo model of endometriosis the tested vitamin D compound significantly reduced total lesion weight.

The data therefore demonstrates the potential for the use of vitamin D compounds in the prevention and treatment of endometriosis.

### Example 2

### Materials and Methods

### In Vivo Model of Endometriosis

Balb/c donor mice were injected with estrogen (Estradiol AMSA; 3 ug/mouse) and one week later were sacrificed and the uterus was removed, the two horns isolated and reduced to small fragments. The fragments derived from the isolated uterine horns were resuspended in saline with ampicillin (1 mg/ml) and then injected into the peritoneum of two recipient Balb/c mice, previously anesthetised, through a 0.5 cm incision in the abdominal wall. Antibiotic (ampicillin 1 mg/ml) was administered on the day of the surgery and on the day after. Starting four hours after the surgery, one mouse in each pair was injected with test compound and the other with control, ip once a day, 5 days a week for two weeks. Dosage levels of the test compounds were at the maximum tolerated levels for the compound in question, i.e. 1-alpha-fluoro-25-hydroxy-16,23E-diene-26,27-bishomo-20-epi-cholecalciferol (Compound A) 100 ug/kg, calcitriol (Compound B) 0.3 ug/kg and 1,25-dihydroxy-21-(3-hydroxy-3-methylbutyl)-19-nor-cholecalciferol (Compound C) 3 ug/kg.

After two weeks, mice were given a lethal dose of anaesthetic and their abdomen was opened to check for lesion presence. Lesions can be identified as translucid isolated or grouped cysts mainly found on the abdominal wall, on the pancreas, and around the uterus. In some cases the lesions are necrotic. The lesions were carefully removed and put on a glass slide to dry for 48 hours, and then were weighed.

### Results

FIG. 4 illustrates the effect of treatment using 1-alpha-fluoro-25-hydroxy-16,23E-diene-26,27-bishomo-20-epi-cholecalciferol (Compound A) versus treatment using the control (vehicle only). FIG. 4A presents the entire data set for 24 mice in each group. FIG. 4B presents the data as the average lesion weight in treated and untreated mice (mean and standard error are shown). FIG. 4C shows the relative reduction in lesion weight between treated and control groups (mean and standard error are shown). Lesion weight reduction between paired animals was calculated: Compound A at 100 ug/kg is able to reduce lesion weight by 51±11% (mean ± standard error) when given for two weeks after uterus transfer (mean lesion weight: 8.452±1.039 mg vs 3.527±0.5400 mg in miglyol and Compound A treated animals respectively). Statistical analysis shows a significant reduction in lesion weight for those mice receiving treatment with the vitamin D analogue Compound A (p=0.0001 for unpaired t test, p=0.0001 for paired t test).

FIG. 5 illustrates the effect of treatment using calcitriol (Compound B) versus treatment using the control (vehicle only). FIG. 5A presents the entire data set for 7 mice in each group. FIG. 5B presents the data as the average lesion weight in treated and untreated mice (mean and standard error are shown). FIG. 5C shows the relative reduction in lesion weight between treated and control groups (mean and standard error are shown). Statistical analysis again shows a significant reduction in lesion weight for mice receiving treatment with a vitamin D analogue, in this case Compound B (p=0.0207 for unpaired t test, p=0.0252 for paired t test).

FIG. 6 illustrates the effect of treatment using 1,25-dihydroxy-21-(3-hydroxy-3-methylbutyl)-19-nor-cholecalciferol (Compound C) versus treatment using the control (vehicle only). FIG. 6A presents the entire data set for 9 mice in each group. FIG. 6B presents the data as the average lesion weight in treated and untreated mice (mean and standard error are shown). FIG. 6C shows the relative reduction in lesion weight between treated and control groups (mean and standard error are shown). Statistical analysis shows no significant reduction in lesion weight for those mice receiving treatment with the vitamin D analogue Compound C (p=0.1122 for unpaired t test, p=0.0781 for paired t test).

### Effect of Vitamin D Compounds

Example 2 demonstrates that a range of vitamin D compounds may be utilised in the present invention. Each of the three test compounds leads to a reduction in lesion weight, although this is most pronounced following treatment with Compound A (which may be administered at a higher dosage level than the other compounds tested, due to its lower associated toxicity).

### Example 3

### Materials and Methods

### Dose/Response Analysis

Balb/c donor mice were injected with estrogen (Estradiol AMSA; 3 ug/mouse) and one week later were sacrificed and the uterus was removed, the two horns isolated and reduced to small fragments. The fragments derived from the isolated uterine horns were resuspended in saline with ampicillin (1 mg/ml) and then injected into the peritoneum of recipient Balb/c mice, previously anesthetised, through a 0.5 cm incision in the abdominal wall. Antibiotic (ampicillin 1 mg/ml) was administered on the day of the surgery and on the day after. Starting four hours after the surgery, each mouse was injected with a specific dose of Compound A or with control, ip once a day, 5 days a week for two weeks

After two weeks, mice were given a lethal dose of anaesthetic and their abdomen was opened to check for lesion presence. Lesions can be identified as translucid isolated or grouped cysts mainly found on the abdominal wall, on the pancreas, and around the uterus. In some cases the lesions are necrotic. The lesions were carefully removed and put on a glass slide to dry for 48 hours, and then were weighed. At least 10 test animals were used in each group.

### Treatment Regimes

Further experiments were performed using Compound A at 100 ug/kg but varying the time at which administration of the vitamin D compound was initiated and the time point at which administration was ceased. Specifically: (i) administration for 1 week prior to injection of the uterine fragments (ii) administration for 2 weeks subsequent to injection of the uterine fragments (iii) administration for 1 week prior and 2 weeks subsequent to injection of the uterine fragments (iv) administration for 2 weeks, initiated two days subsequent to injection of the uterine fragments (v) administration for 2 weeks, initiated two weeks subsequent to injection of the uterine fragments. In these experiments, subjects were sacrificed at the later of two weeks post injection or the end of the treatment period as appropriate.

### Results

The effect of four different doses of Compound A up to the maximum tolerated dose of 100 ug/kg is shown in FIG. 7. The mean and standard error is indicated. The results follow a typical dose/response profile, with greater reduction in lesion weight resulting from higher doses of the test compound. Of note is the fact that lesion weight is reduced at dosages levels well below the maximum tolerated dose (i.e. by approximately 20% at 1/10 MTD and approximately 35% at around ⅓ MTD).

FIG. 8 illustrates the effect of different treatment timings on the reduction in lesion weight. Advance treatment with Compound A, group (i), led to a 40% in lesion weight after two weeks. Treatment with Compound A for two weeks starting at the time of uterus transfer, group (ji), demonstrated a 48% of reduction in lesion weight. The maximum effect was obtained by treating animals for three weeks, one week before and two weeks after uterus transfer (group (iii)), leading to 73% reduction in lesion weight. Compound A is still effective when treatment of animals is initiated 2 days (group (iv), 35% reduction) or 2 weeks (group (v), 34% reduction) after uterus transfer when endometriotic cysts are well established.

Effect of vitamin D compounds

Compound A is effective in treating endometriosis in a mouse model, even at dosages well below the maximum tolerated dose (above which the compound becomes hypercalcemic). Furthermore, Compound A may be expected to be of use in both the treatment and/or prevention of the disorder, based on the fact that pre-treatment and post-treatment both lead to lower lesion weight, with the greatest reduction observed where pre- and post-treatment with Compound A is given.

### Example 4

### Materials and Methods

### Cell Adhesion

Paired animals were treated wth Compound A (100 ug/kg) orally once a day, for two days. The animals were then sacrificed and uterus horns were removed. Myometrium was removed by scraping with a scalpel blade and remaining endometrial tissue was reduced to small fragments with scissors.

Tissue was minced into small pieces (1 to 2 mm³) and incubated at 37° C. for 1 h with 0.1% type A collagenase. At the end of the incubation, single stromal cells were separated from large dumps of epithelium by a 10 min. period of differential sedimentation at unity gravity. The top 8 ml of medium, containing predominantly stromal cells, were then slowly removed and the cells were collected by centrifugation. The stromal-enriched fraction was washed twice in culture medium and allowed to adhere selectively to tissue culture dishes for 15 min. Thereafter, non-attached epithelial cells still present were removed and a purified stromal preparation was obtained on the surface of the culture dishes.

Polystyrene 96-well plates (Costar) were coated with 50 ul/well of 8 mg/ml extracellular matrix (ECM) (Sigma, USA), and left uncovered in a laminar flow hood overnight to allow evaporation. The plates were then rinsed with PBS and used for the attachment assays. Cells were washed three times with PBS, trypsinized and seeded into 200 ul cells at a density of 2×10⁵/ml on ECM. After 1 to 2 h of incubation at 37° C., the wells were gently rinsed three times with PBS to remove unattached cells. The remaining cells in 96-well plates were tested with CyQuant cell proliferation kit (Molecular Probes). The sample fluorescence in each well was measured using a fluorescence microplate reader with filters appropriate for 480 nm excitation and 520 nm emission maxima. Results were expressed as the percentage of total cells assuming that the adhesion of cells in the control was 100%. The percentage of adhesion was determined using the formula: (Abs after being rinsed with PBS/Abs no rinse)×100%. The experiments were performed in triplicate.

### Cell Chemotaxis Assay

Human stromal cell preparation: tissue was gently minced into small pieces (1 to 2 mm³) and incubated at 37° C. for 1 h with 0.1% type A collagenase. At the end of the incubation, single stromal cells were separated from large dumps of epithelium by a 10 min. period of differential sedimentation at unity gravity. The top 8 ml of medium, containing predominantly stromal cells, were then slowly removed and the cells were collected by centrifugation. The stromal-enriched fraction was washed twice in culture medium and allowed to adhere selectively to tissue culture dishes for 15 min. Thereafter, non-attached epithelial cells still present were removed and a purified stromal preparation was obtained on the surface of the culture dishes.

Endometrial stromal cells migration was evaluated by means of chemotaxis experiments in a 48-well modified Boyden chamber. With the migration assay, we assessed the ability of the cells to migrate toward a chemo-attractant on a two-dimensional substrate (in our case, collagen type IV). Briefly, the chemotaxis experiments were performed using 8 um Nuclepore polyvinylpyrrolidine-free polycarbonatefilters coated with 10 ug/ml of type IV collagen and placed over a bottom chamber containing 20 ng/ml PDGF and/or 1 uM estrogen as the chemo-attractant factor. Serum-free medium was used as a negative control. Suspended in D-MEM medium containing 0.1% fatty acid-free bovine serum albumin, the ESC cells were pretreated for 30 min with Compound A at 1 uM and then cells were treated with β-Estradiol for 24 h. After the treatment cells were added to the upper chamber at a density of 4×10⁴ cells/well. After six hours of incubation at 37° C., the non-migrated cells on the upper surface of the filter were removed by scraping. The cells that had migrated to the lower side of the filter were stained with Diff-Quick stain (VWR Scientific Products, Bridgeport, N.J.), and 5-8 unit fields per filter were counted at 160× magnification using a Zeiss microscope. The assays were run in triplicate.

### ELISA Quantification of Cytokine Produced by Peritoneal Macrophages

Peritoneal cells were recovered two weeks after unterus transfer in cold PBS, 2 mM EDTA, by peritoneal lavage of treated (Compound A at 100 ug/kg) and untreated (vehicle only) animals prepared according to the procedure described previously in Examples 1 to 3 (pool of 5 mice per group). Peritoneal macrophages were counted directly after collection using Turk reagent, washed, and placed into culture with RPMI/glutamax 5% FC I, pen/strep, Na pyruvate. After 2 hr at 37° C. the non adherent cells were removed and the macrophages were cultured for a further 48 hr. The supernatant was harvested and cytokines (TNF-alpha, IL1-alpha, IL1-beta, IL6, MIP-2 and VEGF) were quantified using a specific ELISA (R&D System DuoSet). All ELISA determinations were performed in duplicate on the undiluted sample. The total cells number plated was assessed by CyQuant test and values of protein production were normalized to cell number.

### Results

### Cell Adhesion

Compound A is able to dramatically reduce the adhesion of endometriotic cells to collagen, as shown in FIG. 9 (mean and standard error are shown for a total of 5 subjects per group).

### Cell Chemotaxis Assay

FIG. 10 demonstrates that Compound A is able to reduce estrogen induced chemotaxis of human stromal endometrial cells. No effect of Compound A is evident on the basal condition of migration, compared to the approximately 50% of reduction in migration seen with estrogen stimulation.

### ELISA Quantification

FIG. 11 shows that inflammatory cytokine and VEGF production is dramatically reduced by Compound A, suggesting an anti-inflammatory mechanism contributes to this endometriosis mouse model.

### Effect of Vitamin D Compounds

Among the different possible mechanisms of action Compound A on endometriotic lesions there is a direct effect on adhesion and chemotactic responsiveness of endometrial cells. Compound A is able to reduce both the number of adherent cells and can decrease the chemotactic migration of endometrial cells in response to estrogen.

Other possible mechanisms of action for vitamin D compounds include the inhibition of inflammation. Peritoneal macrophages' inflammatory response is well documented to sustain the progression of endometriosis in humans. Consequently we tested the hypothesis that vitamin D compounds, such as Compound A, can modulate peritoneal inflammation in the mouse model of endometriosis and demonstrated that inflammatory cytokine and VEGF production is dramatically reduced by Compound A (FIG. 11). Nonetheless the same macrophages are still capable of producing the same cytokines if re-activated in vitro with a non related stimulus such as LPS (data not shown).

### FORMULATION EXAMPLES

### Formulation Example 1

### Oral Dosage Form Soft Gelatin Capsule

A capsule for oral administration is formulated under nitrogen in amber light from 0.01 to 25.0 mg of Compound A (1-alpha-fluoro-25-hydroxy-16,23E-diene-26,27-bishomo-20-epi-cholecalciferol) in 150 mg of fractionated coconut oil (e.g. Miglyol 812), with 0.015 mg butylated hydroxytoluene (BHT) and 0.015 mg butylated hydroxyanisole (BHA), filled in a soft gelatin capsule.

The capsule is prepared by the following process:
1. BHT and BHA are suspended in fractionated coconut oil (e.g. Miglyol 812) and warmed to around 50° C. with stirring, until dissolved.
2. Compound A is dissolved in the solution from step 1 at 50° C.
3. The solution from step 2 is cooled to room temperature.
4. The solution from step 3 is filled into soft gelatin capsules.

All manufacturing steps are performed under a nitrogen atmosphere and protected from natural light.

### Formulation Example 2

### Oral Dosage Form Soft Gelatin Capsule

A capsule for oral administration is formulated under nitrogen in amber light: 150 μg of Compound A in 150 mg of fractionated coconut oil (Miglyol 812), with 0.015 mg butylated hydroxytoluene (BHT) and 0.015 mg butylated hydroxyanisole (BHA), filled in a soft gelatin capsule.

### Formulation Example 3

### Oral Dosage Form Soft Gelatin Capsule

A capsule for oral administration is formulated under nitrogen in amber light: 75 μg of Compound A in 150 mg of fractionated coconut oil (Miglyol 812), with 0.015 mg butylated hydroxytoluene (BHT) and 0.015 mg butylated hydroxyanisole (BHA), filled in a soft gelatin capsule.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. (canceled)

2. (canceled)

3. A method for preventing and/or treating endometriosis comprising administering to a subject in need thereof a therapeutically effective amount of a vitamin D compound of the formula: 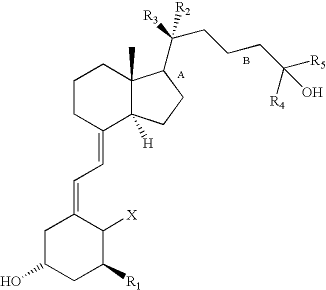
wherein:
X is H₂ or CH₂
R₁ is hydrogen, hydroxy or fluorine
R₂ is hydrogen or methyl
R₃ is hydrogen or methyl provided that when R₂ or R₃ is methyl, R₃ or R₂ must be hydrogen
R₄ is methyl, ethyl or trifluoromethyl
R₅ is methyl, ethyl or trifluoromethyl
A is a single or double bond
B is a single, E-double, Z-double or triple bond.

4. A pharmaceutical composition comprising a therapeutically effective amount for use in the prevention and/or treatment of endometriosis of a vitamin D compound and a pharmaceutically acceptable carrier.

5. The pharmaceutical composition of claim 4, which is packaged with instructions for use in the prevention and/or treatment of endometriosis.

6. (canceled)

7. A kit containing a vitamin D compound together with instructions directing administration of said compound to a patient in need of treatment and/or prevention of endometriosis thereby to treat and/or prevent endometriosis in said patient.

8. The method according to claim 3, wherein the vitamin D compound is administered separately, sequentially or simultaneously in separate or combined pharmaceutical formulations with a second medicament for the treatment of endometriosis.

9. The method according to claim 3, wherein said vitamin D compound is a compound of the formula: 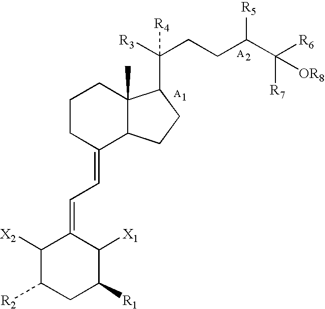
wherein:
A₁ is single or double bond;
A₂ is a single, double or triple bond;
Xi and X₂ are each independently H or ═CH₂, provided X₁ and X₂ are not both ═CH₂;
Ri and R₂ are each independently OC(O)CrC₄ alkyl, OC(O)hydroxyalkyl or OC(O)haloalkyl;
Ri and/or R₂ can alternatively be OH;
R₃, R₄ and R₅ are each independently hydrogen, d-C₄ alkyl, hydroxyalkyl, or haloalkyl, or
R₃ and R₄ taken together with C₂₀ form C₃-C₆ cycloalkyl; and
R₆ and R₇ are each independently C₁₋₄alkyl or haloalkyl; and
R₈ is H, —COCrC₄alkyl, —CO hydroxyalkyl or —COhaloalkyl; and pharmaceutically acceptable esters, salts, and prodrugs thereof.

10. The method according to claim 3, wherein said vitamin D compound is a compound of the formula: 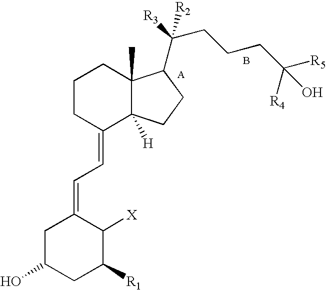
wherein:
X is H₂ or CH₂
Ri is hydrogen, hydroxy or fluorine
R₂ is hydrogen or methyl
R₃ is hydrogen or methyl provided that when R₂ or R₃ is methyl, R₃ or R₂ must be hydrogen
R₄ is methyl, ethyl or trifluoromethyl
R₅ is methyl, ethyl or trifluoromethyl
A is a single or double bond
B is a single, E-double, Z-double or triple bond

11. The method according to claim 10, wherein each of R₄ and R₅ is methyl or ethyl.

12. The method according to claim 3, wherein said vitamin D compound is 1,25-dihydroxy-21-(3-hydroxy-3-methylbutyl)-19-nor-cholecalciferol, having the formula:

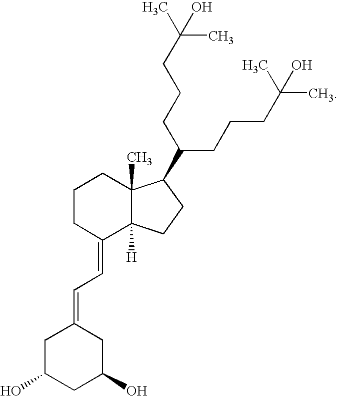

13. The method according to claim 3, wherein said vitamin D compound is 1-alpha-fluoro-25-hydroxy-16,23E-diene-26,27-bishomo-20-epi-cholecalciferol, having the formula:

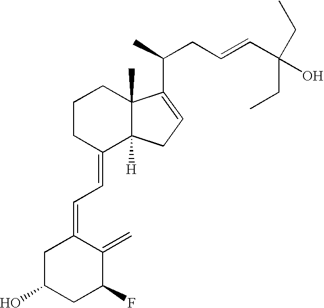

14. The method according to claim 3, wherein said compound is calcitriol.

15. The method according to claim 3, wherein said endometriosis is associated with the presence of symptoms of chronic pelvic pain and/or sub-fertility.

16. The pharmaceutical composition of claim 4, wherein said vitamin D compound is a compound of the formula: 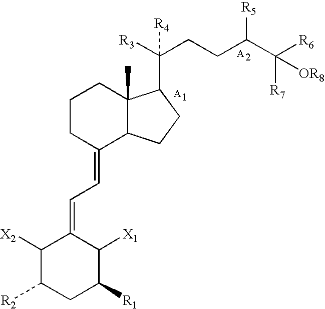
wherein:
A₁ is single or double bond;
A₂ is a single, double or triple bond;
Xi and X₂ are each independently H or ═CH₂, provided Xi and X₂ are not both ═CH₂;
Ri and R₂ are each independently OC(O)CrC₄ alkyl, OC(O)hydroxyalkyl or OC(O)haloalkyl;
Ri and/or R₂ can alternatively be OH;
R₃, R₄ and R₅ are each independently hydrogen, d-C₄ alkyl, hydroxyalkyl, or haloalkyl, or
R₃ and R₄ taken together with C₂₀ form C₃-C₆ cycloalkyl; and
R₆ and R₇ are each independently C₁₋₄alkyl or haloalkyl; and
R₈ is H, —COCrC₄alkyl, —CO hydroxyalkyl or —COhaloalkyl; and pharmaceutically acceptable esters, salts, and prodrugs thereof.

17. The pharmaceutical composition of claim 4, wherein said vitamin D compound is a compound of the formula: 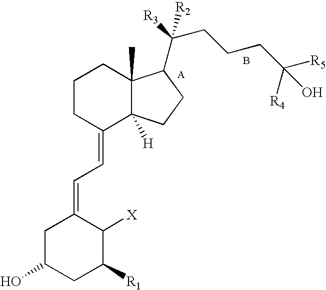
wherein:
X is H₂ or CH₂
Ri is hydrogen, hydroxy or fluorine
R₂ is hydrogen or methyl
R₃ is hydrogen or methyl provided that when R₂ or R₃ is methyl, R₃ or R₂ must be hydrogen
R₄ is methyl, ethyl or trifluoromethyl
R₅ is methyl, ethyl or trifluoromethyl
A is a single or double bond
B is a single, E-double, Z-double or triple bond

18. The pharmaceutical composition of claim 17, wherein each of R₄ and R₅ is methyl or ethyl.

19. The pharmaceutical composition of claim 4, wherein said vitamin D compound is 1,25-dihydroxy-21-(3-hydroxy-3-methylbutyl)-19-nor-cholecalciferol, having the formula:

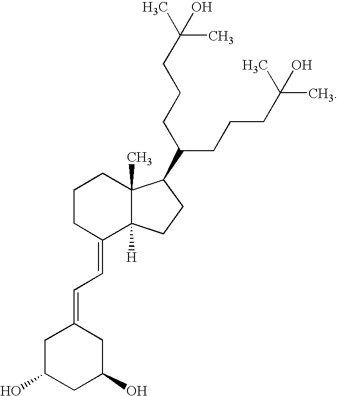

20. The pharmaceutical composition of claim 4, wherein said vitamin D compound is 1-alpha-fluoro-25-hydroxy-16,23E-diene-26,27-bishomo-20-epi-cholecalciferol, having the formula:

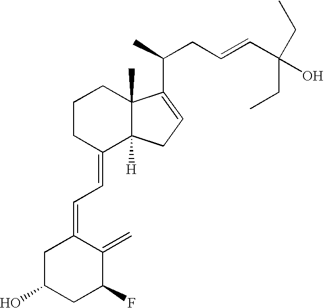

21. The pharmaceutical composition of claim 4, wherein said compound is calcitriol.

22. The kit of claim 7, wherein the vitamin D compound is administered separately, sequentially or simultaneously in separate or combined pharmaceutical formulations with a second medicament for the treatment of endometriosis.

23. The kit of claim 7, wherein said endometriosis is associated with the presence of symptoms of chronic pelvic pain and/or sub-fertility.
